(19) United States
(12) Patent Application Publication
(10) Pub. No.: US 2024/0174639 A1
(43) Pub. Date: May 30, 2024

(54) PYRIDINE DERIVATIVES USEFUL AS HCN2 MODULATORS

(71) Applicant: King's College London, London (GB)

(72) Inventors: Peter A. McNaughton, London (GB); Karen Williams, High Peak (GB); Sue Cramp, Harlow (GB); Alan Naylor, Cambridge (GB)

(73) Assignee: King's College London, London (GB)

(21) Appl. No.: 18/279,796

(22) PCT Filed: Mar. 2, 2022
(86) PCT No.: PCT/GB22/50555

§ 371 Date: Aug. 31, 2023

(30) Foreign Application Priority Data
Mar. 3, 2021 (GB) 2103017.6

(51) Int. Cl. C07D 401/14; A61K 31/4439; A61K 31/444; A61P 27/00; A61P 29/00; C07D 401/10; C07D 471/04

(52) U.S. Cl. CPC C07D 401/14; A61K 31/4439; A61K 31/444; A61P 27/00; A61P 29/00; C07D 401/10; C07D 471/04

(57) ABSTRACT

Compounds of the formula (I) and pharmaceutically acceptable salts thereof: (I) wherein the substituents are defined in the specification. The compounds are hyperpolarisation activated cyclic-nucleotide modulated ion channel 2 (HCN2) inhibitors. Also disclosed are pharmaceutical compositions comprising the compounds, and the use of the compounds for the treatment or prevention of medical conditions mediated by HCN2, including neuropathic pain.

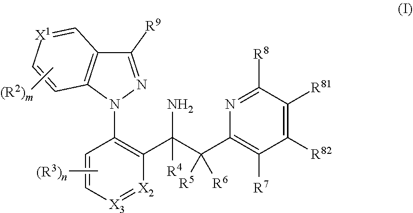

This invention relates to indazole compounds, to pharmaceutical compositions comprising the compounds, and to the use of the compounds for the treatment of medical conditions mediated by hyperpolarisation activated cyclic-nucleotide modulated ion channel 2 (HCN2), for example for the treatment of pain, particularly the treatment of inflammatory and/or neuropathic pain.

### BACKGROUND

Nociception is the ability to detect potentially harmful stimuli to the body resulting from the internal or external stimuli, such as extreme temperatures or tissue injury, and is generated by the activation of nociceptors. The nociceptors transmit information to the brain where the perception of acute pain is generated. Nociception is an important sense that warns an individual against present or imminent damage resulting in an acute pain signal. However, in patients with chronic pain, this warning signal persists in the absence of any genuine threat and can impose major limitations on lifestyle and working patterns. Pain results in around 40 million physician visits per year, approximately 4 billion lost working days, and a dramatic reduction in the quality of life for many patients.

Inflammatory pain (IP) results from the increased excitability of peripheral nociceptive sensory fibres produced by the action of inflammatory mediators released from injured, inflamed or stressed tissues onto nociceptive (pain-sensing) nerve terminals. IP may be chronic or acute. Acute IP is associated with the immediate inflammatory response following tissue damage or injury and includes, for example, post-operative pain, dental pain and injury such as sprains or muscle tears. Generally acute IP resolves as the injury heals. However, IP can also be chronic. Chronic IP is a feature of many medical conditions, for example infection, injury, osteoarthritis and rheumatoid arthritis.

IP is typically treated with non-steroidal anti-inflammatory drugs (NSAIDs) or in more severe cases with opioids, both of which are effective but have major side effects. Undesirable side-effects associated with NSAIDs include gastric and renal complications, together with an increased incidence of myocardial infarction. Side effects associated with opioids include constipation and CNS side effects, for example cognitive impairment, sedation and addiction. Additionally, even at normal doses opiates promote respiratory depression and are the cause of many premature deaths

Neuropathic pain (NP), a form of chronic pain caused by damage to and/or dysfunction of sensory nerves of the peripheral or sympathetic nervous system, for example a lesion or disease of the somatosensory system, including peripheral fibres (AR, Ab and C fibres) and central neurons. The damage to the somatosensory system results in disordered transmission of sensory signals to the brain resulting in the generation of pain. Symptoms of neuropathic pain include abnormal sensation of painful and other stimuli, known as dysesthesia (e.g. hyperesthesia, hyperalgesia, allodynia (pain due to a non-noxious stimulus), and hyperpathia) and/or ongoing pain, typically sensed as deep and aching pain. NP is often long-lasting and typically persists after apparent resolution of the primary cause.

An estimated 50 million patients world-wide suffer from chronic non-malignant pain, defined as pain of greater than 3 months' duration that is not related to cancer. Neuropathic pain affects about 8% of people in the Western World at some point in their life.

Painful diabetic neuropathy (PDN), the pain resulting from nerve damage caused by Type 2 diabetes, is a major patient burden which is rapidly growing with the increasing incidence of obesity and has no highly efficacious treatment options at this stage. Post-herpetic neuralgia (PHN), a long-lasting pain following a Herpes zoster (shingles) eruption, is also a significant problem, particularly amongst the elderly. Pain caused either by cancer or by the chemotherapeutic agents used to treat it (chemotherapy-induced peripheral neuropathy, CIPN) imposes an additional patient burden, and the ability of patients to tolerate the neuropathic pain induced by chemotherapy is often a limiting factor in treatment. Post-operative neuropathic pain sometimes occurs following surgical procedures causing patients chronic pain that may persist long after the surgical wound has healed. In addition to these major patient groups there are many rarer but excruciating neuropathic pain conditions such as trigeminal neuralgia, complex regional pain syndrome (CRPS) and pudendal neuralgia. In addition, many clinicians believe, on the basis that drugs used to treat neuropathic pain have some efficacy in these conditions, that there is a neuropathic pain component in many common conditions involving nerve damage or compression, such as lower back pain, nerve damage following traumatic injury (e.g. whiplash injury in car crash), fibromyalgia and carpal tunnel syndrome.

Existing therapies for NP, such as gabapentinoids, serotonin, noradrenaline-selective reuptake inhibitors (SNRIs) and tricyclic antidepressants, have poor efficacy, with as many as 70% of patients reporting limited or no relief and with the number needed to treat to obtain 50% relief in a single patient (NNT) typically in the range 7-10 (Finnerup, N. B. et al., 2015, Lancet Neurol. 14, 162-173). There are also numerous side effects associated with existing therapies for NP. For example, gabapentin, the current first-line therapy for NP, causes sedation, while amitriptyline (a tricyclic antidepressant) has psychotropic effects such as sedation, nightmares, impotence and confusion together with numerous drug-drug interactions.

There remains a need for new treatments for pain, particularly IP and NP.

The Hyperpolarization activated, Cyclic-Nucleotide modulated (HCN) ion channels comprise four isoforms, HCN 1, 2, 3 and 4, which carry an inward current called Iₕ (also known as I_{q} or I_{f}) activated by hyperpolarization in the range of membrane potentials between −60 and −90 mV (Kaupp & Seifert (2001) “Molecular diversity of pacemaker ion channels.” Annu. Rev. Physiol. 63: 235-257; Biel et al., (2002) “Cardiac HCN channels: structure, function, and modulation.” Trends Cardiovasc. Med. 12(5): 206-212).

The HCN isoforms perform an important pacemaker function in both cardiac and nervous tissue.

HCN4 is the major regulator of cardiac rhythmicity. Inducible deletion of cardiac HCN4 causes a progressive decrease in heart rate which is fatal in mice after a few days (Baruscotti et al., “Deep bradycardia and heart block caused by inducible cardiac-specific knockout of the pacemaker channel gene HCN4”; Proc. Natl. Acad. Sci. USA 108, 2011, 1705-1710). HCN2 is expressed in atrial and ventricular cardiac tissue but appears to be largely excluded from the pacemaker region, the sino-atrial node, in both animals and humans (Herrmann S, Layh B & Ludwig A. “Novel insights into the distribution of cardiac HCN channels: an expression study in the mouse heart”. J. Mol. Cell. Cardiol. 51, 997-1006, 2011; Herrmann S, Hofmann F, Stieber J & Ludwig A. “HCN channels in the heart: lessons from mouse mutants”. Br. J. Pharmacol. 166, 501-509, 2012; Chandler, N. J., et al. “Molecular architecture of the human sinus node: insights into the function of the cardiac pacemaker.” Circulation 119(12): 1562-1575, 2009). The role of HCN2 is also thought to be less critical than HCN4, because the cardiac function of both an HCN2 global knockout mice and a human HCN2 deletion mutant is relatively normal suggesting that HCN2-selective blockers will not cause bradycardia (Ludwig et al. “Absence epilepsy and sinus dysrhythmia in mice lacking the pacemaker channel HCN2”, EMBO J 22, 2003, 216-224; and DiFrancesco et al., “Recessive loss-of-function mutation in the pacemaker HCN2 channel causing increased neuronal excitability in a patient with idiopathic generalized epilepsy”; J Neurosci. 31, 2011, 17327-17337).

HCN1 and HCN2 are the predominant isoforms expressed in both brain and somatosensory neurons (Ludwig et al 2003, ibid).

NP has traditionally been attributed to sensitisation and/or remodelling of the CNS. However, in more recent work it has been shown by the use of peripherally restricted blockers of HCN ion channels and by recordings of activity in single nociceptors (pain-sensitive nerve fibers) that pain continues to have its origin in repetitive firing of peripheral nociceptors even long after the initial injury has apparently resolved. These findings suggest that peripherally restricted blockers of HCN ion channels would provide a new class of analgesics. (Young et al., “Inflammatory and neuropathic pain are rapidly suppressed by peripheral block of hyperpolarisation-activated cyclic nucleotide-gated ion channels”; Pain. 155; 2014, 1708-19; Noh, S., et al. (2014). “The heart-rate-reducing agent, ivabradine, reduces mechanical allodynia in a rodent model of neuropathic pain.” Eur. J. Pain 18(8): 1139-1147; Serra, J., et al. (2012), “Microneurographic identification of spontaneous activity in C-nociceptors in neuropathic pain states in humans and rats.” Pain 153(1): 42-55; reviewed in Tsantoulas, C., et al. (2016). “HCN2 ion channels: basic science opens up possibilities for therapeutic intervention in neuropathic pain.” Biochem. J. 473(18): 2717-2736).

The negative range of activation of HCN ion channels means that they are hardly activated at the resting membrane potential of nerve fibres, which seldom exceeds −60 mV. However, many inflammatory mediators, amongst them the potent pro-inflammatory agents PGE2 and bradykinin, bind to Gs-coupled GPCRs which thus activate adenylate cyclase and so cause an increase in cAMP (cyclic adenosine monophosphate), which in turn binds directly to a site in the C-terminal domain of HCN ion channels. The voltage range of activation of the HCN2 and HCN4 isoforms, but not HCN1 and HCN3, is shifted in the positive direction by increased intracellular cAMP. The inward current passing through activated HCN2 ion channels in nociceptive nerve fibres therefore triggers repetitive firing, resulting in a sensation of pain in vivo (Emery et al., “HCN2 ion channels play a central role in inflammatory and neuropathic pain”; Science 333, 2011, 1462-1466).

A number of studies have shown increased HCN2 channel expression and/or In current in nociceptors following neuronal damage or inflammation, though other studies have failed to find a change in expression or even found a decrease (reviewed in Tsantoulas, C., et al. (2016), ibid). Increased inward Iₕ current is expected to shift the membrane potential to more depolarized values, and so lower the activation threshold. Upregulation of HCN2 has been demonstrated in cell bodies and terminals of nociceptive neurons in preclinical models of inflammatory pain, in line with an increase in Iₕ current and hyperexcitability of the neurons. The same is not true for neuropathic pain models, where there are reports showing no change, or a reduction in HCN ion channel expression (Chaplan S R, Guo H Q, Lee D H, Luo L, Liu C, Kuei C, Velumian A A, Butler M P, Brown S M & Dubin A E., 2003, Neuronal hyperpolarization-activated pacemaker channels drive neuropathic pain. J. Neurosci. 23, 1169-1178; Tsantoulas et al., 2017, ibid). However, there are alternative routes to enhanced Iₕ current than channel overexpression, such as increases in intracellular cAMP, as outlined above (reviewed in Tsantoulas et al., 2016, ibid).

It has been shown that HCN2 is expressed in nociceptive (pain-sensitive) neurons, and that modulation of the voltage-dependence of HCN2 by inflammatory mediators such as PGE2 is a major contributor to IP. It has also been shown in mouse models for inflammatory pain (including pain elicited by injection of PGE2, carrageenan and formalin) that blockage and/or targeted genetic deletion of HCN2 provides analgesia (Emery et al. 2011, ibid).

In a study in a chronic constriction injury (CCI) mouse model of NP in which HCN2 had been genetically deleted from nociceptors, the mice showed no sign of NP following a nerve lesion (Emery et al., 2011 ibid.). Subsequent studies have shown that ivabradine, a non-selective blocker of HCN ion channels, is an effective analgesic in a variety of mouse models of neuropathic pain, including nerve injury, cancer chemotherapy and diabetic neuropathy models (Young et al., 2014, ibid; Tsantoulas et al., 2017, ibid). Further evidence for the central role of HCN2 ion channels in animal pain models is set out in Emery et al., “HCN2 ion channels: an emerging role as the pacemakers of pain” Trends Pharmacol. Sci. 33(8): 2012, 456-463; and Tsantoulas et al., Hyperpolarization-activated cyclic nucleotide-gated 2 (HCN2) ion channels drive pain in mouse models of diabetic neuropathy. Sci Transl. Med 9, 2017, eaam6072. This work suggests that HCN2-selective blockers will provide effective treatments for NP and IP.

The analgesia observed in these mouse models was achieved by blocking or genetically deleting HCN2 ion channels in peripheral nociceptive neurons alone, because the blockers used were peripherally restricted and the targeted genetic deletion was restricted to peripheral nociceptive neurons. In mouse models global genetic deletion of all HCN2, in contrast, caused epilepsy, failure to gain weight and early death (Ludwig et al. Int. J. Mol. Sci. 2015 January; 16(1): 1429-1447). Thus, a peripherally restricted HCN2 blocker is expected to provide an effective analgesic for NP and IP whilst also avoiding CNS mediated side effects which may be associated with blocking HCN2 channels in the brain. The avoidance or minimisation of CNS side-effects would also address a major problem with other analgesics such as opioids and gabapentinoids. Selective HCN2 blockers may also avoid some or all of the undesirable gastric, renal and cardiac side effects associated with NSAIDs or the constipation caused by opiates.

Experiments (Tsantoulas C et al. 2017 ibid) have shown that ivabradine and nociceptor-targeted genetic deletion of HCN2 both give complete analgesia in a mouse model of diabetic neuropathy, which closely mimics the human condition. These experiments demonstrate that neuropathic pain is primarily peripheral in origin, because in each case the intervention was peripheral, as ivabradine is peripherally restricted and the HCN2 genetic deletion was targeted to peripheral nociceptors. The view that peripheral HCN2 block will provide effective analgesia contrasts with the prevailing belief that NP, in particular, is a CNS phenomenon which would require a CNS-penetrant therapy to treat NP.

Several non-selective HCN ion channel blockers are known including ZD7288, zatebradine, cilobradine, KW-3407, YM758 and ivabradine. These compounds were developed primarily as bradycardic agents (Romanelli et al. Current Topics in Medicinal Chemistry, 16:1764-1791 and Postea et al. Nature Reviews Drug Discovery 10, 2011, 903-914).

The non-selective and peripherally restricted HCN blocker, ivabradine, has been approved by the FDA to treat symptoms associated with stable angina and heart failure. HCN4 and HCN1 channels, the targets of ivabradine in these conditions, are critical for the regulation of heart rate, and the mode of action of ivabradine is to cause bradycardia by blocking HCN4 and HCN1, and thereby to reduce the oxygen demand of the heart. Thus, although the studies described above have shown that ivabradine provides an analgesic effect on NP, the compound is not suitable as an analgesic in the clinic, because of its effects on cardiac pacemaking associated with HCN4 and/or HCN1 inhibition. Accordingly, preferred analgesics targeting HCN2 ion channels for the treatment of, for example, pain should not interact to any significant extent with HCN4 and/or HCN1 to avoid or minimise cardiac side-effects such as bradycardia.

WO02/100408 discloses a method for treating neuropathic pain using a compound that decreases the current mediated by an HCN pacemaker channel in a sensory cell. This document focuses on modulation of HCN1 and HCN3 and discloses ZD7288, ZM-227189, Zatebradine, DK-AH268, alinidine, and ivabradine as possible analgesic agents.

WO97/40027 discloses certain benzisoxazole and benzimidazole compounds which are stated to be useful in the treatment of various psychotic conditions.

WO99/18941 claims the use of Iₕ modulators for the treatment of psychiatric disorders.

WO2011/003895 discloses certain benzisoxazole compounds which are substituted by a carboxamide group at the 5, 6, or 7-position on the benzisoxazole ring. The compounds are stated to be Iₕ channel blockers that may be useful in the treatment of neuropathic pain or inflammatory pain. This reference states that compounds disclosed in the earlier filed WO97/40027 and WO99/18941 have a high CNS penetration resulting in undesirable side effects compared to the carboxamide substituted compounds claimed in WO2011/003895.

WO2011/000915 discloses certain zatebradine derivatives which are stated to selectively inhibit one or more HCN isoforms.

WO2011/019747 discloses certain propofol derivatives stated to be useful as HCN channel modulators for the treatment of chronic pain.

There remains a need for HCN channel inhibitors, particularly compounds which selectively inhibit HCN2 channels.

Tinnitus is the conscious perception of sound heard in the absence of physical sound sources external to the body. Tinnitus commonly manifests itself as ringing, buzzing, whistling or hissing sounds in the ear. Tinnitus is estimated to occur in 25.3% of American adults with 7.9% experiencing it frequently (Shargorodsky et al., Prevalence and characteristics of tinnitus among US adults. Am. J. Med. 2010 August; 123(8):711-8). Tinnitus can severely affect quality of life, by, for example, affecting sleep and the ability to concentrate and perform intellectual tasks. It can also lead to anxiety, depression and in extreme cases, suicide.

Tinnitus can be triggered by a number of factors including exposure to loud noise, presbyacusis, ear or head injuries, ear infections, tumours which impact on auditory nerves and certain diseases of the ear (e.g. Meniere's disease). Tinnitus is also a known side-effect of certain drugs, for example, salicylates (e.g. mesalamine or aspirin, particularly when taken in high doses), quinine anti-malarial agents, aminoglycoside antibiotics, certain chemotherapies, particularly platinum cytotoxic agents (e.g. cisplatin, carboplatin and oxaliplatin) and loop diuretics (e.g. furosemide, ethacrynic acid and torsemide). Tinnitus is also associated with auditory dysfunctions such as hyperacusis, distortion of sounds, misophonia, phonophobia and central auditory processing disorders.

There are no drug therapies currently approved by the FDA for the treatment of tinnitus and there is, therefore, an unmet medical need for an effective treatment of the condition.

The inventors have demonstrated for the first time that HCN2 inhibitors, are effective in the treatment of tinnitus using animal models for the condition. Tinnitus is generally considered to be a CNS phenomenon originating in the brain and resulting in referred noise in the ear (Henry et al. Underlying Mechanisms of Tinnitus: Review and Clinical Implications; J. Am. Acad. Audiol. 2014 January; 25(1): 5-126). It was therefore expected that a CNS-penetrant therapy would be required to treat tinnitus. Contrary to this expectation, the Examples herein show that the peripherally restricted HCN blocker, ivabradine and peripherally restricted HCN2 inhibitors of the present invention, provide an effective treatment for tinnitus in an in-vivo model for the condition. These results suggest that a peripherally restricted HCN2 inhibitor may provide an effective treatment of tinnitus and related conditions such as Meniere's disease with the additional benefit of a reduced risk of CNS related side-effects resulting from HCN2 inhibition in the brain.

### BRIEF SUMMARY OF THE DISCLOSURE

In accordance with the present inventions there is provided a compound of the formula (I), or a pharmaceutically acceptable salt thereof:

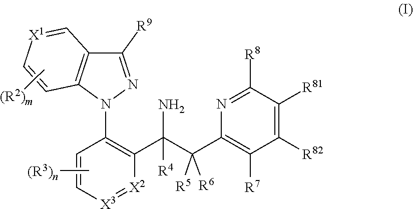

wherein
X¹ is N or CR¹;
   R¹ is selected from: H, halo, —CN, C₁₋₆ alkyl, C₁₋₆ haloalkyl, —OR^{B1}, C₂₋₆ alkenyl, C₂₋₆ alkynyl, C₃₋₆ cycloalkyl and C₃₋₆ cycloalkyl-C₁₋₆ alkyl-, and wherein any alkyl, alkenyl, alkynyl or cycloalkyl group in R¹ is optionally substituted with 1 to 4 substituents independently selected from halo, C₁₋₄ alkyl, C₁₋₄ haloalkyl and —OR^{B2};
R² is independently at each occurrence selected from: halo, C₁₋₆ alkyl and C₁₋₆ haloalkyl;
X² is N or CR³²;
X³ is N or CR³³;
   R³² and R³³ are independently selected from: H, halo, —CN, C₁₋₆ alkyl, C₁₋₆haloalkyl, —NR^{A3}R^{A3} and —OR^{B3};
R³ is independently at each occurrence selected from: halo, —CN, C₁₋₆ alkyl, C₁₋₆ haloalkyl, —NR^{A3}R^{A3} and —OR^{B3};
R⁴, R⁵ and R⁶ are each independently selected from: H and C₁₋₄ alkyl,
   or R⁵ and R⁶ together with the carbon atom to which they are attached form a C₃₋₆ cycloalkyl;
R⁷ is selected from: H, halo, —CN, C₁₋₆ alkyl, C₁₋₆ haloalkyl, C₃₋₆ cycloalkyl, —C(O)NR^{A4}R^{A4}, —N(R^{A4})C(O)R^{B4} and —C(O)R^{B4} and;
R⁸ is independently at each occurrence selected from: H, halo, —CN, nitro, C₁₋₆ alkyl, C₁₋₆ haloalkyl, C₂₋₆alkenyl, C₂₋₆ alkynyl, —OR¹⁰, —NR¹⁰R¹, —S(O)ₓR¹⁰, —C(O)R¹⁰, —OC(O)R¹⁰, —C(O)OR^{10A}, —C(O)NR¹⁰R¹¹, —N(R¹¹)C(O)R¹⁰, —N(R¹¹)C(O)NR¹⁰R¹¹, —N(R¹¹)C(O)OR¹⁰, —N(R¹¹) SO₂R¹⁰, —SO₂NR¹⁰R¹¹, C₃₋₆ cycloalkyl, 3 to 7 membered heterocyclyl, phenyl and 5 or 6 membered heteroaryl; wherein said alkyl, alkenyl, alkynyl, cycloalkyl, or heterocyclyl group is optionally substituted with from 1 to 4 R¹² groups, and said phenyl or heteroaryl group is optionally substituted with from 1 to 4 R¹³ groups;
R⁸¹ and R⁸² are each independently selected from: H, halo, —CN, C₁₋₄ alkyl, C₁₋₄ haloalkyl, —OH and —OR^{B8};
R⁹ is selected from H, halo, —CN and C₁₋₆ alkyl;
R¹⁰ is independently at each occurrence selected from: H, C₁₋₆ alkyl, C₁₋₆ haloalkyl and C₃₋₆ cycloalkyl; wherein said alkyl or cycloalkyl group is optionally substituted with from 1 to 4; R¹⁴ groups;
R^{10A} is selected from: C₁₋₆ alkyl, C₁₋₆ haloalkyl and C₃₋₆ cycloalkyl; wherein said alkyl or cycloalkyl group is optionally substituted with from 1 to 4 R¹⁴ groups;
R¹¹ is independently at each occurrence selected from: H and C₁₋₆ alkyl;
   or R¹⁰ and R¹¹ together with the nitrogen to which they are attached form a 4 to 7 membered heterocyclyl, wherein said heterocyclyl is optionally substituted with 1 or 2 substituents selected from halo, ═O, C₁₋₄ alkyl, C₁₋₄ haloalkyl and —OR^{B7};
R¹² and R¹⁴ are each independently at each occurrence selected from: halo, ═O, —CN, nitro, C₁₋₄ alkyl, C₁₋₄ haloalkyl, C₃₋₆ cycloalkyl, —OR^{B5}, —NR^{A5}R^{A5}, —S(O)ₓR^{B5}, —C(O)R^{B5}, —NR^{A5}C(O)R^{B5}, —C(O)NR^{A5}R^{A5}, —NR^{A5}SO₂R^{B5}, and —SO₂NR^{A5}R^{A5};
R¹³ is independently at each occurrence selected from: halo, —CN, nitro, C₁₋₄ alkyl, C₁₋₄ haloalkyl, C₃₋₆ cycloalkyl, —OR^{B6}, —NR^{A6}R^{A6}, —S(O)ₓR^{B6}, —C(O)R^{A6}, —NR^{A6}C(O)R^{B6}, —C(O)NR^{A6}R^{A6}, —NR^{A6}SO₂R^{B6}, —SO₂NR^{A6}R^{A6};
R^{B1} is independently at each occurrence selected from: H and C₁₋₆ alkyl;
R^{B3} are independently at each occurrence selected from: H, C₁₋₆ alkyl and C₁₋₆ haloalkyl;
R^{B2}, R^{B4}, R^{B5}, R^{B6}, R^{B7} are independently at each occurrence selected from: H, C₁₋₄ alkyl and C₁₋₄ haloalkyl;
R^{B8} is independently at each occurrence selected from: C₁₋₄ alkyl and C₁₋₄ haloalkyl;
R^{A3}, R^{A4}, R^{A5} and R^{A6} are independently at each occurrence selected from H and C₁₋₄ alkyl;
m is an integer selected from: 0, 1, 2 and 3;
n is an integer selected from: 0, 1 or 2; and
x is independently at each occurrence an integer selected from 0, 1, 2 and 3; provided that:
(i) when X¹ is N, then X₂ is CR³² and X₃ is CR³³;
(ii) when X¹ is CR¹ and R¹ is —CN, then X₂ is CR³² and X₃ is CR³³;
(iii) when X¹ is CR¹ and R¹ is —CF₃, then R⁸ is not —SO₂Me; and
(iv) X² and X³ are not both N.

Also provided is a pharmaceutical composition comprising a compound of the invention and a pharmaceutically acceptable excipient.

Also provided is a compound of the invention, or a pharmaceutically acceptable salt thereof, or a pharmaceutical composition of the invention, for use as a medicament. In some embodiments there is provided a compound of the invention, is for use in the treatment of a disease or medical condition mediated by HCN2.

Also provided is a method of treating a disease or medical condition mediated by HCN2 in a subject, the method comprising administering to the subject an effective amount of a compound of the invention or a pharmaceutical composition of the invention.

In some embodiments a compound of the invention is for use in treatment of pain, including neuropathic pain and/or inflammatory pain. In some embodiments a compound of the invention is for use in the treatment of neuropathic pain, particularly chronic neuropathic pain. In some embodiments a compound of the invention is for use in the treatment of peripheral neuropathic pain, particularly chronic peripheral neuropathic pain. In some embodiments a compound of the invention is for use in the treatment of inflammatory pain, particularly chronic inflammatory pain.

A further aspect provides an HCN2 inhibitor for use in the treatment of tinnitus or a related condition. In some embodiments of this aspect, the HCN2 inhibitor is a peripherally restricted HCN2 inhibitor, for example ivabradine. In some embodiments the HCN2 inhibitor in this aspect is a compound of the invention. Preferably the HCN2 inhibitor is a peripherally restricted compound of the invention. Accordingly, also provided is a compound of the invention for use in the treatment or prevention of tinnitus or a related condition (e.g. Meniere's disease or hyperacusis).

### BRIEF DESCRIPTION OF FIGURES

FIG. 1A illustrates the HCN1 and HCN2 voltage step protocol used in Example 50A. FIG. 1B illustrates the HCN4 voltage step protocol used in Example 50A.

FIG. 2 illustrates the HCN current amplitudes in accordance with Example 50B.

FIG. 3 illustrates the voltage protocol used in the measurement of hERG signal in accordance with Example 51A.

FIG. 4 illustrates the voltage protocol used in the measurement of hNaᵥ1.5 signal in accordance with Example 52A.

FIG. 5 shows the effect on tinnitus by pharmacological block of HCN2 ion channels using the gap induced inhibition of the acoustic startle (GPIAS) test of Example 54.

FIG. 6 illustrates the effect of HCN ion channel block on behavioural signs of tinnitus in a short-term (salicylate) model in accordance with Example 54.

FIG. 7 illustrates the effect of HCN ion channel block on behavioural signs of tinnitus in a noise-exposure model in accordance with Example 54.

FIG. 8 illustrates the effect of genetic deletion of HCN2 on auditory brainstem response (ABR) thresholds to tone pulses in accordance with Example 56. The open circle data points in FIG. 8 are from the auditory-targeted HCN2 deletion mice. The shaded data points are from the WT mice.

FIG. 9 illustrates the mechanical analgesic effect of the compound of Example 2 in a mouse neuropathic pain model tested using a von Frey filament. The compound of Example 2 showed full analgesia at an i.p. dose of 0.2 mg/kg. The effects are shown relative to vehicle (“Veh”) and ivabradine (“IVA”) dosed at 5 mg/kg i.p. Significance over vehicle injection shown in the figure (*, p<0.05). The mechanical pain threshold on the y-axis is shown normalised relative to baseline prior to partial sciatic nerve ligation (PSNL), which was carried out 5 days prior to testing the compounds in the model.

FIG. 10 shows the effect of ivabradine i.p. dose on heart rate (left axis, solid circles) and inflammatory pain (right axis, open circles) in a formalin model of inflammatory pain in Black6 mice.

FIG. 11 shows the effect of the compound of Example 2 dosed i.p. in an amount of 0.05, 0.1 and 0.2 mg/kg ((A) in the figure) and 0.5, 1 and 2 mg/kg ((B) in the figure)) relative to ivabradine (“IVA”) 5 mg/kg i.p. and a vehicle control (“Veh”) on heart beat in Black6 mice.

FIG. 12 shows the mechanical analgesic effect of the compound of Example 4 in a mouse neuropathic pain model tested using a von Frey filament. The compound was tested at 2 mg/kg i.p. ((A) in Figure) and 10 mg/kg i.p. ((B) in Figure), relative to ivabradine (“IVA”) dosed at 5 mg/kg i.p. and a vehicle control(“Veh”). The mechanical pain threshold on the y-axis is shown normalised relative to baseline prior to partial sciatic nerve ligation (PSNL), which was carried out 5 days prior to testing the compounds in the model. The horizontal dotted line shows the mechanical pain threshold in in contralateral (un-operated) paw.

FIG. 13 shows the effects of the compound of Example 4 dosed at 2 mg/kg i.p. on bradycardia as a % from baseline heart rate in Black6 mice. The compound was compared with ivabradine (“IVA”) dosed at 5 mg/kg i.p. and a vehicle control (“Veh”).

### DETAILED DESCRIPTION

### Definitions

Unless otherwise stated, the following terms used in the specification and claims have the following meanings set out below.

As used herein “HCN2” designates the “hyperpolarization activated cyclic nucleotide gated potassium and sodium channel 2”. A reference sequence of full-length human HCN2 mRNA transcript is available from the GenBank database under accession number NM_001194, version NM_001194.3.

The terms “a compound of the invention”, “HCN2 inhibitor of the invention”, “HCN2 blocker of the invention” or the like refers to a compound of the Formulae (I), (II), (III), (IV), (V), (VI), (VII), (VIII) or (IX), or a pharmaceutically acceptable salt, solvate, or salt of a solvate thereof, including any of the Examples listed herein.

The terms “treating” or “treatment” refers to any indicia of success in the treatment or amelioration of a disease, pathology or condition, including any objective or subjective parameter such as abatement; remission; diminishing of symptoms or making the pathology or condition more tolerable to the patient; slowing in the rate of degeneration or decline; making the final point of degeneration less debilitating; improving a patient's physical or mental well-being. For example, certain methods herein treat pain, particularly inflammatory pain and/or neuropathic pain by decreasing a symptom of the pain. The term “treating” and conjugations thereof, include prevention of a pathology, condition, or disease (e.g. preventing the development of one or more symptoms of inflammatory pain or neuropathic pain.

The term “associated” or “associated with” in the context of a substance or substance activity or function associated with a disease of condition means that the disease or condition is caused by (in whole or in part), or a symptom of the disease or condition is caused by (in whole or in part) the substance or substance activity or function. For example, a symptom of a disease or condition associated with HCN2 channel activity may be a symptom that results (entirely or partially) from an increase in the level of activity of HCN2 channels or an increase in the expression of the channels. As used herein, what is described as being associated with a disease, if a causative agent, could be a target for treatment of the disease. For example, a disease associated with an increase in the level of activity of a HCN2 channel, may be treated with an agent (e.g. compound as described herein) effective for decreasing the level of activity of HCN2 channels.

As defined herein, the term “inhibition”, “inhibit”, “inhibiting”, “block” or “blocking” and the like in reference to an inhibitor of HCN2 means negatively affecting (e.g. decreasing) the level of activity or function of the HCN2 channel (e.g. a component of the HCN2 channel relative to the level of activity or function of channel in the absence of the inhibitor). In some embodiments inhibition refers to reduction of a disease or symptoms of disease (e.g. pain associated with an increased level of activity of HCN2). In some embodiments, inhibition refers to a reduction in the level of channel current. For example, a compound of the invention may bind to the HCN2 channel to block or prevent current flow through the channel or to produce an allosteric effect which acts to inhibit the action of the channel. Thus, inhibition may include, at least in part, partially or totally blocking stimulation, decreasing, preventing, or delaying activation, or inactivating, desensitizing, or down-regulating channel activity or the amount of a channel protein.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

The term “halo” or “halogen” refers to one of the halogens, group 17 of the periodic table. In particular, the term refers to fluorine, chlorine, bromine and iodine. Preferably, the term refers to fluorine, chlorine or bromine.

The term Cₘ₋ₙ refers to a group with m to n carbon atoms.

The term “C₁₋₆ alkyl” refers to a linear or branched hydrocarbon chain containing 1, 2, 3, 4, 5 or 6 carbon atoms, for example methyl, ethyl, n-propyl, iso-propyl, n-butyl, sec-butyl, tert-butyl, n-pentyl and n-hexyl. “C₁₋₄ alkyl” similarly refers to such groups containing up to 4 carbon atoms. Alkylene groups are divalent alkyl groups and may likewise be linear or branched and have two points of attachment to the remainder of the molecule. Furthermore, an alkylene group may, for example, correspond to one of those alkyl groups listed in this paragraph. The alkyl and alkylene groups may be unsubstituted or substituted by one or more substituents. Possible substituents are described below. Substituents for the alkyl group may be halogen, e.g. fluorine, chlorine, bromine and iodine, OH, C₁-C₄ alkoxy. Other substituents for the alkyl group may alternatively be used.

The term “C₁₋₆ haloalkyl”, e.g. “C₁₋₄ haloalkyl”, refers to a hydrocarbon chain substituted with at least one halogen atom independently chosen at each occurrence, for example fluorine, chlorine, bromine and iodine. The halogen atom may be present at any position on the hydrocarbon chain. For example, C₁₋₆ haloalkyl may refer to chloromethyl, fluoromethyl, trifluoromethyl, chloroethyl e.g. 1-chloromethyl and 2-chloroethyl, trichloroethyl e.g. 1,2,2-trichloroethyl, 2,2,2-trichloroethyl, fluoroethyl e.g. 1-fluoroethyl and 2-fluoroethyl, trifluoroethyl e.g. 1,2,2-trifluoroethyl and 2,2,2-trifluoroethyl, chloropropyl, trichloropropyl, fluoropropyl, trifluoropropyl. A haloalkyl group may be a fluoroalkyl group, i.e. a hydrocarbon chain substituted with at least one fluorine atom.

The term “C₂₋₆ alkenyl” includes a branched or linear hydrocarbon chain containing at least one double bond and having 2, 3, 4, 5 or 6 carbon atoms. The double bond(s) may be present as the E or Z isomer. The double bond may be at any possible position of the hydrocarbon chain. For example, the “C₂₋₆ alkenyl” may be ethenyl, propenyl, butenyl, butadienyl, pentenyl, pentadienyl, hexenyl and hexadienyl.

The term “C₂₋₆ alkynyl” includes a branched or linear hydrocarbon chain containing at least one triple bond and having 2, 3, 4, 5 or 6 carbon atoms. The triple bond may be at any possible position of the hydrocarbon chain. For example, the “C₂₋₆ alkynyl” may be ethynyl, propynyl, butynyl, pentynyl and hexynyl.

The term “C₃₋₆ cycloalkyl” includes a saturated hydrocarbon ring system containing 3, 4, 5 or 6 carbon atoms. For example, the “C₃-C₆ cycloalkyl” may be cyclopropyl, cyclobutyl, cyclopentyl, cyclohexyl, bicyclo[2.1.1]hexane or bicyclo[1.1.1]pentane.

The term “heterocyclyl”, “heterocyclic” or “heterocycle” includes a 3- to 7-membered non-aromatic monocyclic or bicyclic saturated or partially saturated group comprising 1, 2 or 3 heteroatoms independently selected from O, S and N in the ring system (in other words 1, 2 or 3 of the atoms forming the ring system are selected from O, S and N). By partially saturated it is meant that the ring may comprise one or two double bonds. This applies particularly to monocyclic rings with from 5 to 7 members. The double bond will typically be between two carbon atoms but may be between a carbon atom and a nitrogen atom. Bicyclic systems may be spiro-fused, i.e. where the rings are linked to each other through a single carbon atom; vicinally fused, i.e. where the rings are linked to each other through two adjacent carbon or nitrogen atoms; or they may be share a bridgehead, i.e. the rings are linked to each other through two non-adjacent carbon or nitrogen atoms. Examples of heterocyclic groups include cyclic ethers such as oxiranyl, oxetanyl, tetrahydrofuranyl, dioxanyl, and substituted cyclic ethers. Heterocycles comprising at least one nitrogen in a ring position include, for example, azetidinyl, pyrrolidinyl, piperidinyl, piperazinyl, morpholinyl, thiomorpholinyl, tetrahydrotriazinyl, tetrahydropyridinyl, homopiperidinyl, homopiperazinyl, 2,5-diaza-bicyclo[2.2.1]heptanyl and the like. Typical sulfur containing heterocycles include tetrahydrothienyl, dihydro-1,3-dithiolane, tetrahydro-2H-thiopyran, and hexahydrothiepine. Other heterocycles include dihydro oxathiolyl, tetrahydro oxazolyl, tetrahydro-oxadiazolyl, tetrahydrodioxazolyl, tetrahydrooxathiazolyl, hexahydrotriazinyl, tetrahydro oxazinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. A suitable value for a heterocyclyl group which bears 1 or 2 oxo (═O), for example, 2 oxopyrrolidinyl, 2-oxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. For example, the terms “piperidinyl” or “morpholinyl” includes a piperidin-1-yl or morpholin-4-yl ring that is linked via the ring nitrogen (i.e. a piperidino or morpholino ring), the term also includes carbon linked rings (e.g. piperidin-4-yl or morpholin-3-yl).

The term “bridged ring systems” includes ring systems in which two rings share more than two atoms, see for example Advanced Organic Chemistry, by Jerry March, 4th Edition, Wiley Interscience, pages 131-133, 1992.

The term “spiro bi-cyclic ring systems” includes ring systems in which two ring systems share one common spiro carbon atom, i.e. the heterocyclic ring is linked to a further carbocyclic or heterocyclic ring through a single common spiro carbon atom.

“Heterocyclyl-Cₘ₋ₙ alkyl” includes a heterocyclyl group covalently attached to a Cₘ₋ₙ alkylene group, both of which are defined herein; and wherein the Heterocyclyl-Cₘ₋ₙ alkyl group is linked to the remainder of the molecule via a carbon atom in the alkylene group. The groups “aryl-Cₘ₋ₙ alkyl” “heteroaryl-Cₘ₋ₙ alkyl” are defined in the same way.

“—Cₘ₋ₙ alkyl substituted by —NRR” and “Cₘ₋ₙ alkyl substituted by —OR” similarly refer to an —NRR or —OR group covalently attached to a Cₘ₋ₙ alkylene group and wherein the group is linked to the remainder of the molecule via a carbon atom in the alkylene group.

Reference to “R¹⁰ and R¹¹ together with the nitrogen to which they are attached form a 4 to 7 membered heterocyclyl” refers to R¹⁰ and R¹¹ being attached to the same nitrogen atom and forming a nitrogen-linked heterocyclyl. By way of example, the group —NR¹⁰R¹¹ may form e.g. a pyrrolidn-1-yl, piperidin-1yl, piperazin-1yl or morpholin-4yl group.

The term “aromatic” when applied to a substituent as a whole includes a single ring or polycyclic ring system with 4n+2 electrons in a conjugated π system within the ring or ring system where all atoms contributing to the conjugated π system are in the same plane.

The term “aryl” includes an aromatic hydrocarbon ring system. The ring system has 4n+2 electrons in a conjugated π system within a ring where all atoms contributing to the conjugated π system are in the same plane. For example, the “aryl” may be phenyl and naphthyl. The aryl system itself may be substituted with other groups.

The term “heteroaryl” includes an aromatic mono- or bicyclic ring incorporating one or more (for example 1-4, particularly 1, 2 or 3) heteroatoms selected from nitrogen, oxygen or sulfur. The ring or ring system has 4n+2 electrons in a conjugated π system where all atoms contributing to the conjugated π system are in the same plane.

The heteroaryl group can be, for example, a 5- or 6-membered monocyclic ring. The ring may contain up to about four heteroatoms typically selected from nitrogen, sulfur and oxygen. Typically, the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general, the number of basic nitrogen atoms present in the heteroaryl group, including any amino group substituents of the ring, will be less than five.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

The term “optionally substituted” includes either groups, structures, or molecules that are substituted and those that are not substituted.

Where optional substituents are chosen from “one or more” groups it is to be understood that this definition includes all substituents being chosen from one of the specified groups or the substituents being chosen from two or more of the specified groups.

Where a moiety is substituted, it may be substituted at any point on the moiety where chemically possible and consistent with atomic valency requirements. The moiety may be substituted by one or more substituents, e.g. 1, 2, 3 or 4 substituents; optionally there are 1 or 2 substituents on a group. Where there are two or more substituents, the substituents may be the same or different.

Substituents are only present at positions where they are chemically possible, the person skilled in the art being able to decide (either experimentally or theoretically) without undue effort which substitutions are chemically possible and which are not.

Ortho, meta and para substitution are well understood terms in the art. For the absence of doubt, “ortho” substitution is a substitution pattern where adjacent carbons possess a substituent, whether a simple group, for example the fluoro group in the example below, or other portions of the molecule, as indicated by the bond ending in “ [symbol] ”.

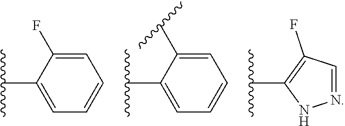

“Meta” substitution is a substitution pattern where two substituents are on carbons one carbon removed from each other, i.e. with a single carbon atom between the substituted carbons. In other words, there is a substituent on the second atom away from the atom with another substituent. For example, the groups below are meta substituted.

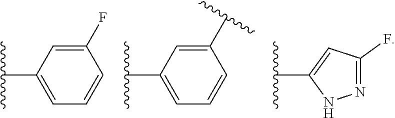

“Para” substitution is a substitution pattern where two substituents are on carbons two carbons removed from each other, i.e. with two carbon atoms between the substituted carbons. In other words, there is a substituent on the third atom away from the atom with another substituent. For example, the groups below are para substituted.

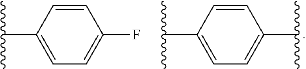

A bond terminating in a “ [symbol] ” or “*” represents that the bond is connected to another atom that is not shown in the structure. A bond terminating inside a cyclic structure and not terminating at an atom of the ring structure represents that the bond may be connected to any of the atoms in the ring structure where allowed by valency.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

The various functional groups and substituents making up the compounds of the present invention are typically chosen such that the molecular weight of the compound does not exceed 1000. More usually, the molecular weight of the compound will be less than 750, for example less than 700, or less than 650, or less than 600, or less than 550. More preferably, the molecular weight is less than 525 and, for example, is 500 or less.

Suitable or preferred features of any compounds of the present invention may also be suitable features of any other aspect.

The invention contemplates pharmaceutically acceptable salts of the compounds of the invention. These may include the acid addition and base salts of the compounds. These may be acid addition and base salts of the compounds.

Suitable acid addition salts are formed from acids which form non-toxic salts. Examples include the acetate, aspartate, benzoate, besylate, bicarbonate/carbonate, bisulfate/sulfate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 1,5-naphthalenedisulfonate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulfate and hemicalcium salts. For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of compounds of the invention may be prepared by for example, one or more of the following methods:
(i) by reacting the compound of the invention with the desired acid or base;
(ii) by removing an acid- or base-labile protecting group from a suitable precursor of the compound of the invention or by ring-opening a suitable cyclic precursor, for example, a lactone or lactam, using the desired acid or base; or
(iii) by converting one salt of the compound of the invention to another by reaction with an appropriate acid or base or by means of a suitable ion exchange column.

These methods are typically carried out in solution. The resulting salt may precipitate out and be collected by filtration or may be recovered by evaporation of the solvent. The degree of ionisation in the resulting salt may vary from completely ionised to almost non-ionised.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterized by the absolute configuration of its asymmetric centre and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”. Where a compound of the invention has two or more stereo centres any combination of (R) and (S) stereoisomers is contemplated. The combination of (R) and (S) stereoisomers may result in a diastereomeric mixture or a single diastereoisomer. The compounds of the invention may be present as a single stereoisomer or may be mixtures of stereoisomers, for example racemic mixtures and other enantiomeric mixtures, and diasteroemeric mixtures. Where the mixture is a mixture of enantiomers the enantiomeric excess may be any of those disclosed above. Where the compound is a single stereoisomer the compounds may still contain other diasteroisomers or enantiomers as impurities. Hence a single stereoisomer does not necessarily have an enantiomeric excess (e.e.) or diastereomeric excess (d.e.) of 100% but could have an e.e. or d.e. of about at least 85%

The compounds of this invention may possess one or more asymmetric centres; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z-isomers). It is to be understood that the present invention encompasses all optical, diastereoisomers and geometric isomers and mixtures thereof that possess HCN2 inhibitory activity.

Z/E (e.g. cis/trans) isomers may be separated by conventional techniques well known to those skilled in the art, for example, chromatography and fractional crystallisation.

Conventional techniques for the preparation/isolation of individual enantiomers when necessary include chiral synthesis from a suitable optically pure precursor or resolution of the racemate (or the racemate of a salt or derivative) using, for example, chiral high pressure liquid chromatography (HPLC) or chiral supercritical fluid chromatography (SFC). Thus, chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and for specific examples, 0 to 5% by volume of an alkylamine e.g. 0.1% diethylamine. Alternatively, when chiral SFC is employed a supercritical fluid, generally CO₂, is used as the mobile phase. The properties of the supercritical fluid may be modified by the inclusion of one or more co-solvents, e.g. an alcohol such as methanol, ethanol or isopropanol, acetonitrile or ethylacetate. Concentration of the eluate affords the enriched mixture.

Alternatively, the racemate (or a racemic precursor) may be reacted with a suitable optically active compound, for example, an alcohol, or, in the case where the compound of the invention contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person. An enantiomer of a compound may also be prepared using a chiral auxiliary during the synthesis of the compound, in which a suitable chiral intermediate is reacted with an intermediate of the compound followed by one or more diastereoselective transformations. The resulting diastereomers are then separated using conventional methods, such as those described above, followed by removal of the chiral auxiliary to provide the desired enantiomer.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, “Stereochemistry of Organic Compounds” by E. L. Eliel and S. H. Wilen (Wiley, 1994).

Compounds and salts described in this specification may be isotopically-labelled (or “radio-labelled”). Accordingly, one or more atoms are replaced by an atom having an atomic mass or mass number different from the atomic mass or mass number typically found in nature. Examples of radionuclides that may be incorporated include ²H (also written as “D” for deuterium), ³H (also written as “T” for tritium), ¹¹C, ¹³C, ¹⁴C, ¹⁵O, ¹⁷O, ¹⁸O ¹³N, ¹⁵N, ¹⁸F, ³⁶Cl, ¹²³I, ²⁵I, ³²P, ³⁵S and the like. The radionuclide that is used will depend on the specific application of that radio-labelled derivative. For example, for in vitro competition assays, ³H or ¹⁴C are often useful. For radio-imaging applications, ¹¹C or ¹⁸F are often useful. In some embodiments, the radionuclide is ³H. In some embodiments, the radionuclide is ¹⁴C. In some embodiments, the radionuclide is ¹¹C. And in some embodiments, the radionuclide is ¹⁸F.

Isotopically-labelled compounds can generally be prepared by conventional techniques known to those skilled in the art or by processes analogous to those described using an appropriate isotopically-labelled reagent in place of the non-labelled reagent previously employed.

The selective replacement of hydrogen with deuterium in a compound may modulate the metabolism of the compound, the PK/PD properties of the compound and/or the toxicity of the compound. For example, deuteration may increase the half-life or reduce the clearance of the compound in-vivo. Deuteration may also inhibit the formation of toxic metabolites, thereby improving safety and tolerability. It is to be understood that the invention encompasses deuterated derivatives of compounds of formula (I). As used herein, the term deuterated derivative refers to compounds of the invention where in a particular position at least one hydrogen atom is replaced by deuterium. For example, one or more hydrogen atoms in a C₁₋₄-alkyl group may be replaced by deuterium to form a deuterated C₁₋₄-alkyl group, for example CD₃.

Certain compounds of the invention may exist in solvated as well as unsolvated forms such as, for example, hydrated forms. It is to be understood that the invention encompasses all such solvated forms or pharmaceutically acceptable salts thereof that possess HCN2 inhibitory activity.

It is also to be understood that certain compounds of the invention may exhibit polymorphism, and that the invention encompasses all such forms that possess HCN2 inhibitory activity.

Compounds of the invention may exist in a number of different tautomeric forms and references to compounds of the invention include all such forms. For the avoidance of doubt, where a compound can exist in one of several tautomeric forms, and only one is specifically described or shown, all others are nevertheless embraced by compounds of the invention. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

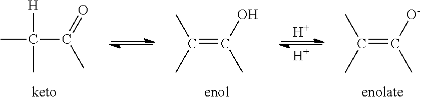

The in vivo effects of a compound of the invention may be exerted in part by one or more metabolites that are formed within the human or animal body after administration of a compound of the invention.

It is further to be understood that a suitable pharmaceutically-acceptable pro-drug of a compound of the formula (I) also forms an aspect of the present invention. Accordingly, the compounds of the invention encompass pro-drug forms of the compounds and the compounds of the invention may be administered in the form of a pro-drug, that is a compound that is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the invention and in-vivo cleavable amide derivatives that may be formed at a carboxy group or an amino group in a compound of the invention.

Accordingly, the present invention includes those compounds of the invention as defined herein when made available by organic synthesis and when made available within the human or animal body by way of cleavage of a pro-drug thereof. Accordingly, the present invention includes those compounds of the formula (I) that are produced by organic synthetic means and also such compounds that are produced in the human or animal body by way of metabolism of a precursor compound, that is a compound of the formula (I) may be a synthetically-produced compound or a metabolically-produced compound.

A suitable pharmaceutically-acceptable pro-drug of a compound of the invention is one that is based on reasonable medical judgement as being suitable for administration to the human or animal body without undesirable pharmacological activities and without undue toxicity.

Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

### Compounds

In some embodiments the compound of the formula (I) is a compound of the formula (II), or a pharmaceutically acceptable salt thereof:

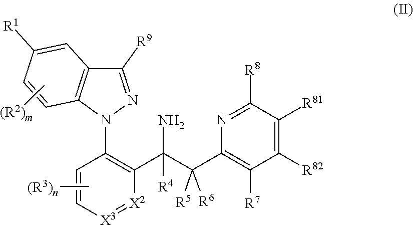

In some embodiments the compound of the formula (I) is a compound of the formula (III), or a pharmaceutically acceptable salt thereof:

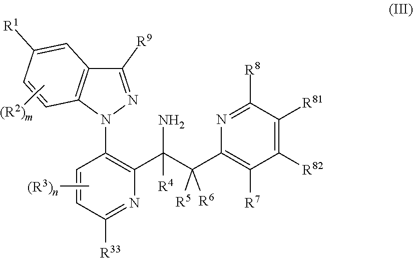

In some embodiments the compound of the formula (I) is a compound of the formula (IV), or a pharmaceutically acceptable salt thereof:

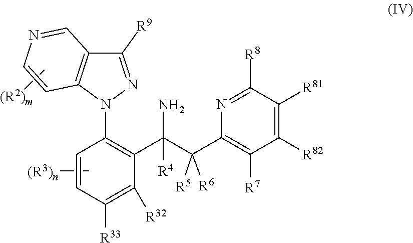

In some embodiments the compound of the formula (I) is a compound of the formula (V), or a pharmaceutically acceptable salt thereof:

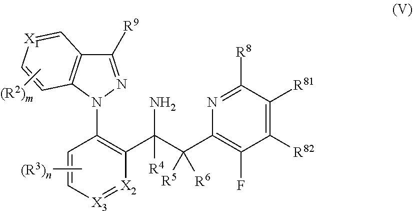

In some embodiments the compound of the formula (I) is a compound of the formula (VI), or a pharmaceutically acceptable salt thereof:

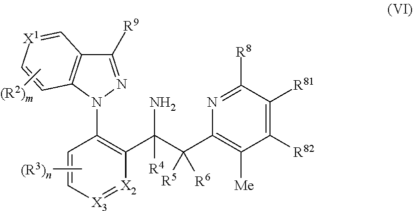

In some embodiments the compound of the formula (I) is a compound of the formula (VII), or a pharmaceutically acceptable salt thereof:

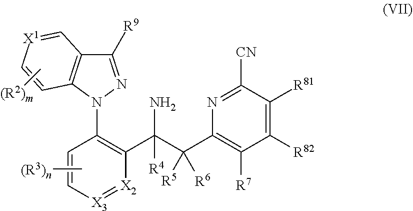

In some embodiments the compound of the formula (I) is a compound of the formula (VIII), or a pharmaceutically acceptable salt thereof:

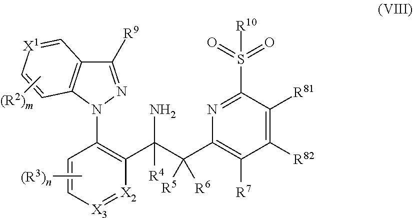

In some embodiments the compound of the formula (I) is a compound of the formula (IX), or a pharmaceutically acceptable salt thereof:

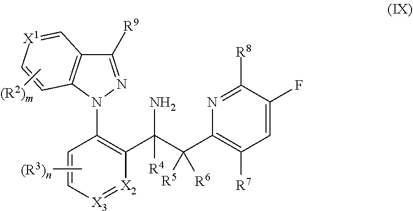

Particular compounds of the invention include, for example, compounds of formulae (I), (II), (III), (IV), (V), (VI), (VII), (VIII) or (IX), or a pharmaceutically acceptable salt thereof, wherein, unless otherwise stated, each of R¹, R², R³, R⁴, R⁵, R⁶, R¹, R⁸, R³², R³³, R⁸¹, R⁸², R⁹, R¹⁰, X₁, X₂, X₃, n and m has any of the meanings defined hereinbefore or in any one or more of paragraphs (1) to (135) hereinafter:
1. X₁ is CR¹ and R¹ is selected from: H, halo, —CN, C₁₋₄ alkyl, C₁₋₄ haloalkyl, —OR^{B1}, C₂₋₄ alkenyl, C₂₋₄ alkynyl, C₃₋₆ cycloalkyl and C₃₋₆ cycloalkyl-C₁₋₃ alkyl-, and wherein said alkyl, alkenyl, alkynyl or a cycloalkyl group in R¹ is optionally substituted with 1 to 4 substituents independently selected from halo, C₁₋₄ alkyl, C₁₋₄ haloalkyl and —OR^{B2}.
2. X₁ is CR¹ and R¹ is selected from: halo, —CN, C₁₋₄ alkyl, C₁₋₄haloalkyl, —OR^{B1}, C₂₋₄ alkenyl, C₂₋₄ alkynyl, C₃₋₆ cycloalkyl and C₃₋₆ cycloalkyl-C₁₋₃ alkyl-, and wherein said alkyl, alkenyl, alkynyl or a cycloalkyl group in R¹ is optionally substituted with 1 to 4 substituents independently selected from halo, C₁₋₄ alkyl, C₁₋₄ haloalkyl and —OR^{B2}.
3. X₁ is CR¹ and R¹ is selected from: halo, —CN, C₁₋₄ alkyl, C₁₋₄ haloalkyl, C₃₋₅ cycloalkyl, C₃₋₅ cycloalkyl-C₁₋₃ alkyl- and wherein said alkyl or cycloalkyl group is optionally substituted with 1 to 4 substituents independently selected from halo, C₁₋₄ alkyl, C₁₋₄ haloalkyl and —OR^{B2}.
4. X₁ is CR¹ and R¹ is selected from: H, halo, —CN, C₁₋₄ alkyl, C₁₋₄ haloalkyl and —OR^{B1} and wherein said alkyl group in R¹ is optionally substituted with —OR^{B2}.
5. X₁ is CR¹ and R¹ is selected from: halo, —CN, C₁₋₄ alkyl, C₁₋₄haloalkyl and —OR^{B1}, and wherein said alkyl group in R¹ is optionally substituted with —OR^{B2}.
6. X₁ is CR¹ and R¹ is selected from: H, F, C, Br, —CN, methyl, ethyl, propyl, isopropyl, cyclopropyl, —CF₃, —CHF₂, —CH₂F, hydroxymethyl, 2-hydroxyethyl, 1-hydroxyethyl, methoxymethyl, 1-methoxyethyl and 2-methoxyethyl.
7. X₁ is CR¹ and R¹ is selected from: H, halo, —CN, C₁₋₃ alky, C₁₋₃ haloalkyl and —OR^{B1}.
8. X₁ is CR¹ and R¹ is selected from: H, halo, —CN, C₁₋₃ alky and C₁₋₃ haloalkyl (e.g. R¹ is selected from: H, halo, —CN, C₁₋₃ alkyl, and —CF₃).
9. X₁ is CR¹ and R¹ is selected from: H, halo, —CN, methyl, ethyl, isopropyl and methoxy.
10. X₁ is CR¹ and R¹ is selected from: H, halo, —CN, methyl and ethyl.
11. X₁ is CH.
12. X₁ is CR¹ and R¹ is —CN.
13. X₁ is CR¹ and R¹ is halo (e.g. F, C or Br).
14. X₁ is CR¹ and R¹ is C₁₋₃alkyl (e.g. methyl).
15. X₁ is CR¹ and R¹ is —CF₃.
16. X₁ is N or CR¹ and R¹ is as defined in any of paragraphs (1) to (15).
17. X₁ is N.
18. R² is independently at each occurrence selected from halo, C₁₋₄ alkyl and C₁₋₄ haloalkyl.
19. R² is selected from halo and C₁₋₃ alkyl.
20. R² is selected from F, Cl, Br and methyl.
21. R² is C₁₋₃ alkyl (e.g. methyl).
22. R² is selected from F, Cl and Br.
23. R² is selected from F and Cl.
24. m is 0.
25. m is 0 and X, is as defined in any of (1) to (15).
26. m is 0 and X, is CH.
27. m is 1.
28. m is 1 and R² is as defined in any of (18) to (23).
29. R³ is independently at each occurrence selected from: halo, C₁₋₄ alkyl and C₁₋₄ haloalkyl.
30. R³ is independently at each occurrence selected from: halo and C₁₋₃ alkyl.
31. R³ is independently at each occurrence selected from: F, Cl, Br, methyl, ethyl and isopropyl.
32. R³ is independently at each occurrence selected from: F, Cl, Br and methyl.
33. n is 0 or 1.
34. n is 0 or 1 and R³ is as defined in any of (29) to (32).
35. n is 1.
36. n is 1 and R³ is as defined in any of (29) to (32).
37. n is 0.
38. X₂ is CR³² and R³² is selected from: halo, C₁₋₄ alkyl and C₁₋₄ haloalkyl.
39. X₂ is CR³² and R³² is selected from: halo and C₁₋₃ alkyl.
40. X₂ is CR³² and R³² is selected from: F, Cl and methyl.
41. X₃ is CR³³ and R³³ is selected from: halo, C₁₋₄ alkyl and C₁₋₄ haloalkyl.
42. X₃ is CR³³ and R³³ is selected from: halo and C₁₋₃ alkyl.
43. X₃ is CR³³ and R³³ is selected from: F, Cl and methyl.
44. X₂ is N, X₃ is CR³³ and R³³ is selected from: halo and C₁₋₃ alkyl.
45. X₂ is CR³² and X₃ is CR³³.
46. X₂ is CR³², X₃ is CR³³, and R³² and R³³ are as defined in any of (38) to (43).
47. X₂ and X₃ are both CH.
48. R⁴, R⁵ and R⁶ are each independently selected from: H and C₁₋₃alkyl,
or R⁵ and R⁶ together with the carbon atom to which they are attached form cyclopropyl.
49. R⁴ is H or methyl.
50. R⁴ is H.
51. R⁵ and R⁶ are each independently selected from: H and C₁₋₃ alkyl.
52. R⁵ is H and R⁶ is selected from: H and C₁₋₃ alkyl.
53. R⁵ is H and R⁶ is C₁₋₃ alkyl (e.g. methyl).
54. R⁵ and R⁶ are H.
55. R⁴ and R⁵ are H and R⁶ is C₁₋₃ alkyl (e.g. methyl or ethyl).
56. R⁴, R⁵ and R⁶ are H.
57. One of R⁴, R⁵ and R⁶ is C₁₋₃ alkyl (e.g. methyl) and other two groups are H.
58. R⁹ is selected from: H, halo, —CN and C₁₋₄ alkyl.
59. R⁹ is selected from: H, —CN and C₁₋₄ alkyl.
60. R⁹ is selected from: H and C₁₋₄ alkyl.
61. R⁹ is H.
62. R⁹ is C₁₋₄ alkyl.
63. R⁹ is methyl, ethyl, propyl or isopropyl.
64. R⁹ is —CN.
65. R⁷ is selected from H, halo, —CN, C₁₋₄ alkyl, C₁₋₄ haloalkyl, C₃₋₅ cycloalkyl, —N(R^{A4})C(O)R^{B4} and —C(O)R^{B4}.
66. R⁷ is selected from halo, —CN, C₁₋₄ alkyl, C₁₋₄ haloalkyl, C₃₋₅ cycloalkyl, —N(R^{A4})C(O)R^{B4} and —C(O)R^{B4}.
67. R⁷ is selected from: —CN, —N(R^{A4})C(O)R^{B4} and C(O)R^{B4}.
68. R⁷ is selected from: H, halo and C₁₋₄ alkyl.
69. R⁷ is C₁₋₄ haloalkyl (e.g. —CF₃, —CH₂F, —CHF₂ or —CH₂CF₃).
70. R⁷ is selected from: halo and C₁₋₃ alkyl.
71. R⁷ is selected from F and methyl.
72. R⁷ is halo (e.g. F).
73. R⁷ is C₁₋₃ alkyl (e.g. methyl or ethyl).
74. R⁷ is H.
75. R³ is independently at each occurrence selected from: H, halo, —CN, C₁₋₄ alkyl, C₁₋₄ haloalkyl, C₂₋₄ alkenyl, C₂₋₄ alkynyl, —OR¹⁰, —NR¹⁰R¹¹, —S(O)ₓR¹⁰ (wherein x is 0, 1 or 2, preferably 1 or 2), —C(O)R¹⁰, —C(O)NR¹⁰R¹¹, —N(R¹¹)C(O)R¹⁰, —N(R¹¹)C(O)NR¹⁰R¹¹, —N(R¹¹)C(O)OR¹⁰, —N(R¹¹) SO₂R¹⁰, —SO₂NR¹⁰R¹¹, C₃₋₆ cycloalkyl, 4 to 6 membered heterocyclyl, phenyl and 5 or 6 membered heteroaryl;
   wherein said alkyl, alkenyl, alkynyl, cycloalkyl, or heterocyclyl group is optionally substituted with from 1 to 4 R¹² groups, and said phenyl or heteroaryl group is optionally substituted with from 1 to 4 R¹³ groups.
76. Ra is selected from: H, halo, —CN, C₁₋₄ alkyl, C₁₋₄haloalkyl, —OR¹⁰, —NR¹⁰R¹¹, —S(O)₂R¹⁰, —C(O)NR¹⁰R¹¹, —N(R¹¹)C(O)R¹⁰, —N(R¹¹)C(O)NR¹⁰R¹¹, —N(R¹¹)C(O)OR¹⁰, —N(R¹¹) SO₂R¹⁰, C₃₋₆ cycloalkyl, 4 to 6 membered heterocyclyl and 5 or 6 membered heteroaryl containing 1 or 2 ring nitrogen atoms;
   wherein said alkyl, cycloalkyl, or heterocyclyl group is optionally substituted with from 1 to 4 R¹² groups and said heteroaryl group is optionally substituted with from 1 to 4 R¹³ groups.
77. R³ is selected from: H, halo, —CN, C₁₋₄ alkyl, C₁₋₄ haloalkyl, —OR¹⁰, —S(O)₂R¹⁰, —C(O)NR¹⁰R¹¹, C₃₋₅ cycloalkyl, 4 to 6 membered heterocyclyl containing 1 ring nitrogen atom and optionally 1 additional ring heteroatom selected from O, S and N, and heteroaryl, wherein said heteroaryl is selected from pyrrolyl, pyrazolyl, imidazolyl, oxazolyl, isoxazolyl, thiazolyl and isothiazolyl;
   wherein said alkyl, cycloalkyl, or heterocyclyl group is optionally substituted with from 1 to 4 R¹² groups and said heteroaryl group is optionally substituted with from 1 to 4 R¹³ groups.
78. R³ is selected from: H, halo, —CN, C₁₋₄ alkyl, C₁₋₄ haloalkyl, —OR¹⁰, —S(O)₂R¹⁰, —C(O)NR¹⁰R¹¹, C₃₋₅ cycloalkyl, 4 to 6 membered heterocyclyl and heteroaryl, wherein said heteroaryl is selected from pyrrolyl, pyrazolyl, imidazolyl, oxazolyl and isoxazolyl, and wherein said 4 to 6 membered heterocyclyl is selected from: azetidinyl, pyrrolidinyl, piperidinyl, piperazinyl, morpholinyl and thiomorpholinyl;
   wherein said alkyl, cycloalkyl, or heterocyclyl group is optionally substituted with from 1 to 4 R¹² groups, and said heteroaryl group is optionally substituted with from 1 to 4 R¹³ groups.
79. R³ is selected from: H, halo, —CN, C₁₋₆ alkyl, C₁₋₆ haloalkyl, —OR¹⁰, —NR¹⁰R¹¹, —S(O)ₓR¹⁰ (wherein x is 0, 1 or 2, preferably 1 or 2), —C(O)NR¹⁰R¹¹, —N(R¹¹)C(O)R¹⁰, —N(R¹¹)C(O)NR¹⁰R¹¹, —N(R¹¹)C(O)OR¹⁰, —SO₂NR¹⁰R¹¹ and —N(R¹¹) SO₂R¹⁰;
   wherein said alkyl group is optionally substituted with from 1 or 2 substituents selected from halo, —CN, —OR^{B5}, —NR^{A5}R^{A5}, —S(O)₂R^{B5}, —C(O)R^{B5}, —NR^{A5}C(O)R^{B5}, —C(O)NR^{A5}R^{A5}, —NR^{A5}SO₂R^{B5} and —SO₂NR^{A5}R^{A5}.
80. R³ is selected from: 4 to 6 membered heterocyclyl and heteroaryl, wherein said heteroaryl is selected from pyrrolyl, pyrazolyl, imidazolyl, oxazolyl and isoxazolyl; and wherein said heterocyclyl is selected from, azetidinyl, pyrrolidinyl, piperidinyl, piperazinyl, morpholinyl and thiomorpholinyl;
   wherein said 4 to 6 membered heterocyclyl group is optionally substituted with from 1 to 4 groups independently selected from halo, C₁₋₄ alkyl, —OH and ═O; and
   wherein said heteroaryl group is optionally substituted with from 1 to 4 groups independently selected from halo, —CN, C₁₋₄ alkyl, C₁₋₄ haloalkyl, —OR^{B6} and —NR^{A6}R^{A6}.
81. R⁸ is selected a 4 or 5 membered heteroaryl, wherein said heteroaryl is optionally substituted by 1 to 4 groups independently selected from halo, —CN, C₁₋₄ alkyl, C₁₋₄ haloalkyl, —OR^{B6} and —NR^{A6}R^{A6}
82. R⁸ is selected from: —CN, —OR¹⁰, —NR¹⁰R¹¹, —S(O)₂R¹⁰, —C(O)NR¹⁰R¹¹, —SO₂NR¹⁰R¹¹, —C₁₋₄ alkyl-CN, —C₁₋₄ alkyl-OR^{B5}, —C₁₋₄ alkyl-NR^{A5}R^{A5}, —C₁₋₄ alkyl-S(O)₂R^{B5}, —C₁₋₄ alkyl-C(O)NR^{A5}R^{A5}, and —C₁₋₄ alkyl-SO₂NR^{A5}R^{A5}.
83. R⁸ is selected from: —CN, —OR¹⁰¹, —NR¹⁰¹R¹¹¹, —S(O)₂R¹¹², —C(O)NR¹⁰¹R¹¹¹, —SO₂NR¹⁰¹R¹¹¹, —C₁₋₄ alkyl-CN, —C₁₋₄ alkyl-OR^{B5}, —C₁₋₄ alkyl-NR^{A5}R^{A5}, —C₁₋₄ alkyl-S(O)₂R^{B5}, —C₁₋₄ alkyl-C(O)NR^{A5}R^{A5}, and —C₁₋₄ alkyl-SO₂NR^{A5}R^{A5}; wherein R¹⁰¹ is selected from: H, C₁₋₃ alkyl and C₁₋₆ cycloalkyl; R¹¹¹ is independently selected from: H and C₁₋₃ alkyl; and R¹¹² C₁₋₄ alkyl.
84. R³ is independently at each occurrence selected from: halo, —CN, C₁₋₄ alkyl, C₁₋₄ haloalkyl, —C₁₋₄ alkyl-OR^{B5}, —C₁₋₃alkyl-C₃₋₆ cycloalkyl, —C₁₋₄ alkyl-NR^{A5}C(O)R^{B5}, —C₁₋₄ alkyl-C(O)NR^{A5}R^{A5}, —C₁₋₄ alkyl-NR^{A5}SO₂R^{B5}, —C₁₋₄ alkyl-SO₂NR^{A5}R^{A5}, —OH, —OC₁₋₄ alkyl, —OC₂₋₄ alkyl-OR^{B5}, —C₂₋₄ alkyl-NR^{A5}R^{A5}, —NH₂, —N(R¹¹)C₁₋₄ alkyl, —N(R¹¹)C₂₋₄ alkyl-OR^{B5}, —N(R¹¹)C₂₋₄ alkyl-NR^{A5}R^{A5}, —S(O)₂C₁₋₄ alkyl, —C(O)NH₂, —C(O)N(R¹)C₁₋₄ alkyl, —C(O)N(R¹)C₁₋₄ alkyl-C₃₋₆ cycloalkyl, —C(O)N(R¹¹)C₂₋₄ alkyl-OR^{B5}, —C(O)N(R¹¹)C₂₋₄ alkyl-NR^{A5}R^{A5}, azetidin-1-yl-C(O)—, pyrrolidin-1-yl-C(O)—, piperidin-1-yl-C(O)—, piperarazin-1-yl-C(O)—, morpholin-1-yl-C(O)—, —N(R¹¹)C(O)—C₁₋₄ alkyl, —N(R¹¹)C(O)NH₂, —N(R¹¹)C(O)NH(C₁₋₄ alkyl), —N(R¹¹)C(O)N(C₁₋₄ alkyl)₂, —N(R¹¹)C(O)O(C₁₋₄ alkyl), —N(R¹¹)SO₂(C₁₋₄ alkyl), C₃₋₆ cycloalkyl, 4 to 6 membered heterocyclyl and heteroaryl,
   wherein said heteroaryl is selected from pyrrolyl, pyrazolyl, imidazolyl, oxazolyl and isoxazolyl,
   wherein said heterocyclyl is selected from, azetidinyl, pyrrolidinyl, piperidinyl, piperazinyl, morpholinyl and thiomorpholinyl;
   wherein said C₃₋₆ cycloalkyl, or 4 to 6 membered heterocyclyl group is optionally substituted with from 1 to 4 groups independently selected from halo, C₁₋₄ alkyl, —OH and ═O; and
   wherein said heteroaryl group is optionally substituted with from 1 to 4 groups independently selected from halo, —CN, C₁₋₄ alkyl, C₁₋₄ haloalkyl, —OR^{B6} and —NR^{A6}R^{A6}.
85. R⁸ is selected from: —C(O)NH₂, —C(O)N(H)C₁₋₃ alkyl and —C(O)N(C₁₋₃ alkyl)₂.
86. R⁸ is selected from: —S(O)₂R¹⁰, for example —S(O)₂C₁₋₄ alkyl or —S(O)₂C₃₋₆ cycloalkyl.
87. R⁸ is selected from —S(O)₂C₁₋₄ alkyl, preferably —S(O)₂Me.
88. R⁸ is selected from: H, halo, —CN, C₁₋₄ alkyl, C₁₋₄ haloalkyl, —C₁₋₄ alkyl-OR^{B5}, —OH, —OC₁₋₄ alkyl, —OC₂₋₄ alkyl-OR^{B5}, —C(O)C₁₋₄ alkyl, —S(O)₂C₁₋₄ alkyl, —C(O)NH₂, —C(O)N(H)C₁₋₄ alkyl and —C(O)N(C₁₋₄ alkyl)₂.
89. R⁸ is selected from: H, halo (e.g. F, Cl, Br), —CN, C₁₋₄ alkyl, —CH₂CH₂—OH, —CH₂CH₂—OMe, —OCH₂CH₂—OH, —OCH₂CH₂—OMe, —S(O)₂Me, —C(O)NH₂, —C(O)N(H)Me and —C(O)N(Me)₂.
90. R⁸ is independently at each occurrence selected from: halo (e.g. F or Br), —CN, methyl, ethyl, methoxy, —S(O)₂Me and —CF₃.
91. R⁸ is independently at each occurrence selected from: halo (e.g. F or Br), —CN, methyl, ethyl and —S(O)₂Me.
92. R⁸ is independently at each occurrence selected from: —CN, methyl, ethyl and —S(O)₂Me.
93. R⁸ is C₁₋₄ alkyl.
94. R⁸ —CN.
95. R⁸ is halo (e.g. F, C or Br).
96. R⁸ is H.
97. R⁸¹ and R⁸² are independently selected from: H, halo, C₁₋₄ alkyl, C₁₋₄ haloalkyl, —OC₁₋₄ alkyl and —OC₁₋₄ haloalkyl.
98. R⁸¹ and R⁸² are independently selected from: H, halo, C₁₋₄ alkyl, —CF₃ and —OC₁₋₄ alkyl.
99. R⁸¹ and R⁸² are independently selected from: H, halo, C₁₋₃ alkyl, —CF₃ and —OCH₃.
100. R⁸¹ is H and R⁸² is selected from: halo, C₁₋₄ alkyl, C₁₋₄ haloalkyl, —OC₁₋₄ alkyl and —OC₁₋₄ haloalkyl.
101. R⁸¹ is H and R⁸² is selected from: halo, C₁₋₄ alkyl, —CF₃ and —OC₁₋₄ alkyl.
102. R⁸¹ is H and R⁸² is selected from: halo (e.g. F), methyl, —CF₃ and —OMe.
103. R⁸¹ is H and R⁸² is halo.
104. R⁸¹ is H and R⁸² is C₁₋₄ alkyl.
105. R⁸² is H and R⁸¹ is selected from: halo, C₁₋₄ alkyl, C₁₋₄ haloalkyl, —OC₁₋₄ alkyl and —OC₁₋₄ haloalkyl.
106. R⁸² is H and R⁸¹ is selected from: halo, C₁₋₄ alkyl, —CF₃ and —OC₁₋₄ alkyl.
107. R⁸² is H and R⁸¹ is selected from: halo (e.g. F), methyl, —CF₃ and —OMe.
108. R⁸² is H and R⁸¹ is halo.
109. R⁸² is H and R⁸¹ is C₁₋₄ alkyl.
110. R⁸¹ is halo.
111. R⁸¹ is H.
112. R⁸² is halo.
113. R⁸² is H.
114. R⁸² and R⁸¹ are H.
115. R⁸¹ is selected from halo, C₁₋₄ alkyl, —CF₃ and —OC₁₋₄ alkyl; R⁸² is H; R⁷ is H, halo, —CF₃ or C₁₋₃ alkyl; and R⁸ is as defined in any of (75) to (96).
116. R⁸¹ is selected from halo, C₁₋₄ alkyl, —CF₃ and —OC₁₋₄ alkyl; R⁸² is H; R⁷ is H or C₁₋₃ alkyl; and R⁸ is as defined in any of (75) to (96).
117. R⁸¹ is halo; R⁸² is H; R⁷ is H or C₁₋₃ alkyl; and R⁸ is as defined in any of (75) to (96).
118. R⁸² is selected from halo, C₁₋₄ alkyl, —CF₃ and —OC₁₋₄ alkyl; R⁸¹ is H; R⁷ is H, halo, —CF₃ or C₁₋₃ alkyl; and R⁸ is as defined in any of (75) to (96).
119. R⁸² is selected from halo, C₁₋₄ alkyl, —CF₃ and —OC₁₋₄ alkyl; R⁸¹ is H; R⁷ is H or C₁₋₃ alkyl; and R⁸ is as defined in any of (75) to (96).
120. R¹⁰ is independently at each occurrence selected from: H and C₁₋₄ alkyl, wherein said alkyl is optionally substituted by halo, —CN, —OR^{B5} and —NR^{A5}R^{A5}.
   R¹¹ is independently at each occurrence selected from: H and C₁₋₄ alkyl;
   or R¹⁰ and R¹¹ together with the nitrogen to which they are attached form a 4 to 6 membered heterocyclyl selected from azetidinyl, pyrrolidinyl, piperidinyl, piperazinyl, morpholinyl and thiomorpholinyl, wherein said heterocyclyl is optionally substituted with 1 or 2 substituents selected from halo, ═O, C₁₋₄ alkyl, C₁₋₄ haloalkyl and —OR^{B7}.
121. R¹⁰ and R¹¹ are independently at each occurrence selected from: H and C₁₋₄ alkyl.
122. When X¹ is CR¹ and R¹ is C₁₋₆ haloalkyl (e.g. —CF₃), then R⁸ is not —SO₂R¹⁰.
123. X¹ is CR¹; R¹ is C₁₋₆ haloalkyl (e.g. —CF₃); and R⁸ is selected from: H, C₁₋₄ alkyl, —OH, —O—C₁₋₄ alkyl, —O—C₂₋₄ alkyl-OH, —O—C₂₋₄ alkyl-OMe, —NH₂, —N(H)C₁₋₄ alkyl, —N(C₁₋₄ alkyl)₂, —C₁₋₄ alkyl-OR^{B5}, —C₁₋₄ alkyl-NR^{A5}R^{A5}, C₃₋₆ cycloalkyl, 4 to 6 membered heterocyclyl and 5 or 6 membered heteroaryl;
   wherein said C₃₋₆ cycloalkyl, or 4 to 6 membered heterocyclyl group is optionally substituted with from 1 to 4 groups independently selected from C₁₋₄ alkyl, —OH and ═O; and
   wherein said heteroaryl group is optionally substituted with from 1 to 4 groups independently selected from C₁₋₄ alkyl, —OR^{B6} and —NR^{A6}R^{A6}.
124. X¹ is CR¹; R¹ is C₁₋₄ haloalkyl (e.g. —CF₃) and R⁸ is selected from: H, C₁₋₄ alkyl, —OH, —O—C₁₋₄ alkyl, —O—C₂₋₄ alkyl-OR^{B5}, and —C₁₋₄ alkyl-OR^{B5}.
125. R¹, R⁸, R⁸¹ and R⁸² are H.
126. R⁷, R⁸, R⁸¹ and R⁸² are H; X, is CR¹ and R¹ is halo.
127. At least one of R⁷, R⁸, R⁸¹ and R⁸² is not H.
128. X₂ is CR³², X₃ is R³³ and at least one of R⁷, R⁸, R⁸¹ and R⁸² is selected from halo, C₁₋₄ alkyl and C₁₋₄ haloalkyl.
129. R⁸¹ is halo (e.g. F) and R⁷ is C₁₋₃ alkyl.
130. R⁷ is selected from C₁₋₃ alkyl and halo; and R⁸ is selected from: H, C₁₋₃ alkyl and —CN.
131. R⁷ is selected from C₁₋₃ alkyl and halo; R⁸ is selected from: H, C₁₋₃ alkyl and —CN; and X₂ is N.
132. X, is CR¹, R¹ is selected from H and halo (e.g. F, C or Br); n is 0; R⁴, R⁵ and R⁶ are H; X₃ is CH; R⁷ is selected from H, F or Me; R⁸ is selected from H, —CN, C₁₋₃ alkyl, 2-hydroxyethyl, 2-methoxyethyl and —S(O)₂C₁₋₃ alkyl; R⁸² is selected from H and F; and R⁸¹ is H.
133. One or more hydrogen atoms in the compound is deuterium.
134. The group

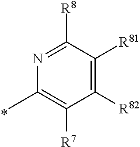

is selected from:

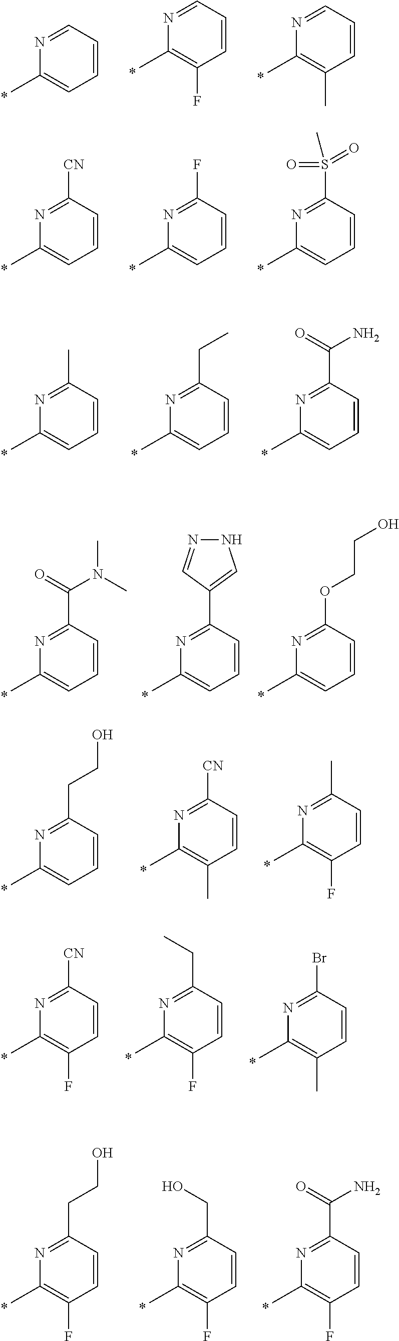

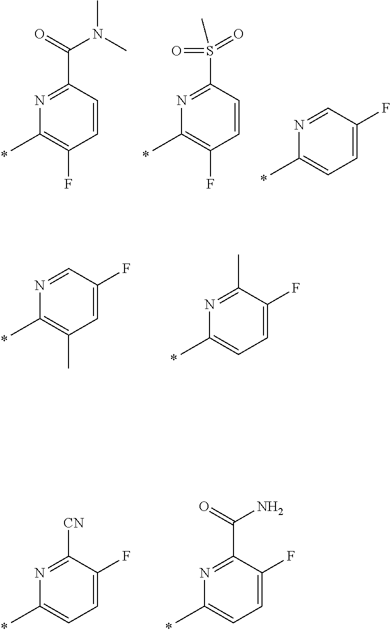

wherein * shows the point of attachment to the remainder of the molecule.
135. The group

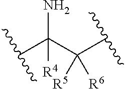

is of the formula

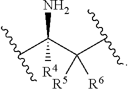

In some embodiments there is provided a compound of the formula (I), or a pharmaceutically acceptable salt thereof wherein:
X₁ is CR¹;
R¹ is selected from H, halo, —CN, C₁₋₄ alkyl and C₁₋₄ haloalkyl; m is 0 or 1;
R² is selected from: halo, C₁₋₄ alkyl and C₁₋₄ haloalkyl;
R⁹ is selected from H and C₁₋₄ alkyl;
X₂ is CH or N;
X₃ is CR³³, wherein R³³ is selected from: H, halo and C₁₋₄ alkyl;
n is 0;
R⁴ and R⁵ are H;
R⁶ is selected from: H and methyl;
R⁸¹ and R⁸² are each independently selected from: H, halo, C₁₋₄ alkyl, C₁₋₄ haloalkyl and —OC₁₋₄ alkyl (e.g. H, halo or C₁₋₃ alkyl);
R⁷ is as defined in any one of paragraphs (65) to (74) above; and
R⁸ is as defined in any one of paragraphs (75) to (96) above.

In this embodiment it may be that R¹ is selected from: H and halo.

In this embodiment it may be that R¹ is H.

In this embodiment it may be that R¹ is halo (e.g. F, C or Br).

In this embodiment it may be that m is 0.

In this embodiment it may be that X₂ is N, X₃ is CR³³, wherein R³³ is selected from H, halo and C₁₋₃ alkyl; and n is 0.

In this embodiment it may be that R⁷ is selected from: H, halo and C₁₋₃ alkyl.

In this embodiment it may be that R⁸ is selected from: H, halo, —CN, C₁₋₄ alkyl, —C₁₋₄ alkyl-OH, —C₁₋₄ alkyl-OMe, —O—C₂₋₄ alkyl-OH, —O—C₂₋₄ alkyl-OMe, —C(O)NH₂, —C(O)N(H)C₁₋₃ alkyl, —C(O)N(C₁₋₃ alkyl)₂ and —S(O)₂C₁₋₄ alkyl.

In this embodiment it may be that R⁸¹ is halo; R⁸² is H; R⁷ is H or C₁₋₃ alkyl; and R⁸ is selected from any one of paragraphs (75) to (96) above (e.g. R⁸ is selected from: H, halo, —CN, C₁₋₄ alkyl, —C₁₋₄ alkyl-OH, —C₁₋₄ alkyl-OMe, —O—C₂₋₄ alkyl-OH, —O—C₂₋₄ alkyl-OMe, —C(O)NH₂, —C(O)N(H)C₁₋₃ alkyl, —C(O)N(C₁₋₃ alkyl)₂ and —S(O)₂C₁₋₄ alkyl).

In this embodiment it may be that X₁ is CR¹; R¹ is selected from H, F, Cl and Br; n is 0; X₂ is CH or N; and X₃ is CH.

In this embodiment it may be that X₁ is CR¹; R¹ is selected from H, F, Cl and Br; n is 0; X₂ is CH or N; X₃ is CH; R⁷ is selected from: halo and C₁₋₃ alkyl; R⁸¹ and R⁸² are H; and R⁸ is selected from any one of paragraphs (75) to (96) above (e.g. R⁸ is selected from: H, —CN, C₁₋₄ alkyl and 2-hydroxyethyl).

In this embodiment it may be that X₁ is CR¹; R¹ is selected from H, F, C and Br; n is 0; X₂ is CH or N; X₃ is CH; R⁷ is selected from: H, halo and C₁₋₃ alkyl; R⁸¹ and R⁸² are H; and R⁸ is selected from: —C₁₋₄ alkyl-OH, —C₁₋₄ alkyl-OMe, —O—C₂₋₄ alkyl-OH, —O—C₂₋₄ alkyl-OMe, —C(O)NH₂, —C(O)N(H)C₁₋₃ alkyl, —C(O)N(C₁₋₃ alkyl)₂ and —S(O)₂C₁₋₄ alkyl (e.g. R⁸ is selected from: halo, —CN, C₁₋₄ alkyl, 2-hydroxyethyl and —S(O)₂Me).

In this embodiment it may be that X₁ is CR¹; R¹ is selected from H, F, C and Br; n is 0; X₂ is CH or N; X₃ is CH; R⁷ is selected from: H, halo and C₁₋₃ alkyl; R⁸¹ is selected from halo and C₁₋₃ alkyl; R⁸² is H; R⁷ is selected from H and C₁₋₃ alkyl; R⁸ is selected from any one of paragraphs (75) to (96) above (e.g. R⁸ is selected from: H, halo, —CN, C₁₋₄ alkyl, —C₁₋₄ alkyl-OH, —C₁₋₄ alkyl-OMe, —O—C₂₋₄ alkyl-OH, —O—C₂₋₄ alkyl-OMe, —C(O)NH₂, —C(O)N(H)C₁₋₃ alkyl, —C(O)N(C₁₋₃ alkyl)₂ and —S(O)₂C₁₋₄ alkyl).

In this embodiment it may be that R⁸¹ is selected from halo, C₁₋₃alkyl, C₁₋₃ haloalkyl and —OC₁₋₃ alkyl; R⁸² is H; R⁸ is selected from —CN and —S(O)₂R¹⁰ (e.g. —S(O)₂C₁₋₄ alkyl); and R⁷ has any of the values in any one of paragraphs (65) to (74) above.

In this embodiment it may be that R⁸² is selected from halo, C₁₋₃ alkyl, C₁₋₃ haloalkyl and —OC₁₋₃ alkyl; R⁸¹ is H; R⁸ is selected from —CN and —S(O)₂R¹⁰ (e.g. —S(O)₂C₁₋₄ alkyl); and R⁷ has any of the values in any one of paragraphs (65) to (74) above.

In another embodiment there is provided a compound of the formula (I), or a pharmaceutically acceptable salt thereof wherein:
R¹ is selected from: H, halo and C₁₋₃ alkyl;
m and n are 0;
R⁹ is selected from: H and C₁₋₄ alkyl (preferably R⁹ is H);
X₂ is selected from: N and CH;
X₃ is CR³³, wherein R³³ is selected from: H, halo and C₁₋₃ alkyl;
R⁴ and R⁵ are H;
R⁶ is selected from: H and methyl; and
the group:

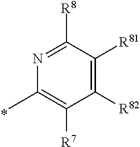

is selected from:

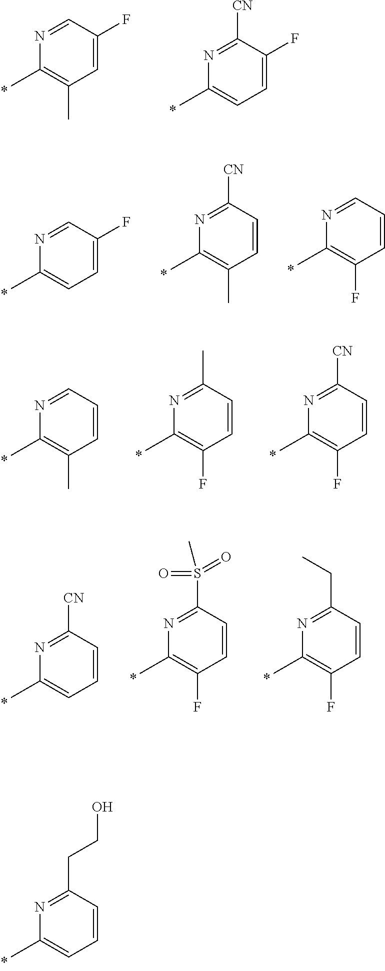

wherein * shows the point of attachment to the remainder of the molecule.

In this embodiment it may be that R¹ is selected from H, F, Cl and Br (e.g. R¹ is H).

In this embodiment it may be that X² is N.

### Compounds of the Formulae (II) and (III)

In some embodiments the compound of formula (I) is a compound of the formula (II), or a pharmaceutically acceptable salt thereof as hereinbefore defined. In the compound of the formula (II) or formula (III) R¹, R², R³, R⁴, R⁵, R⁶, R¹, R⁸, R⁸¹, R⁸², R⁹, X₂, X₃, m and n are as defined in relation to the compound of formula (I), or, unless stated otherwise, have any of the values defined herein including one of more of (1) to (135) (in so far as those paragraphs are applicable to a compound of the formula (II) or (III)). The following embodiments are directed to compounds of the formula (II) or formula (III).

In some embodiments in the compound of the formula (II) or (III), it may be that R⁷ has any of the values in any one of paragraphs (65) to (74) above. It may be that R⁷ is selected from H, halo and C₁₋₃ alkyl.

In some embodiments in the compound of the formula (II) or (III), it may be that R⁸ has any of the values in any one of paragraphs (75) to (96) above. It may be that R⁸ is selected from H, —CN and —S(O)₂R¹⁰.

In some embodiments in the compound of formula (II) or formula (III), including the embodiments above, one of R⁸¹ and R⁸² is H and the other is selected from: halo, C₁₋₄ alkyl, C₁₋₄ haloalkyl and —OC₁₋₄ alkyl.

In some embodiments in the compound of formula (II) or formula (III), one of R⁸¹ and R⁸² is H and the other is selected from: halo, C₁₋₄ alkyl, C₁₋₄ haloalkyl and —OC₁₋₄ alkyl; R⁷ is selected from H, halo and C₁₋₃ alkyl; and R⁸ is selected from —CN and —S(O)₂R¹⁰ (e.g. —S(O)₂C₁₋₄ alkyl).

In some embodiments in the compound of formula (II) or formula (III) R⁸¹ and R⁸² are both H.

In some embodiments in the compound of formula (II) or formula (III), including the embodiments above, R¹ is selected from H, halo, C₁₋₃ alkyl and C₁₋₃ haloalkyl.

In some embodiments in the compound of formula (II) or formula (III), m is 0 and R¹ is selected from H, C₁₋₃ alkyl and —CN.

In some embodiments, including the embodiments above, in the compound of formula (II) or formula (III), R¹ is selected from: halo and C₁₋₃ alkyl.

In some embodiments in the compound of formula (II) or formula (III), including the embodiments above, R¹ is halo (e.g. R¹ is F).

In some embodiments in the compound of formula (II) or formula (III), including the embodiments above, R¹ is H.

In some embodiments in the compound of formula (II) or formula (III), including the embodiments above, R⁷ is selected from H, halo and C₁₋₃ alkyl.

In some embodiments, including the embodiments above, in the compound of formula (II) or formula (III), R⁹ is H.

In some embodiments, including the embodiments above, in the compound of formula (III), R³³ is not H.

In some embodiments, including the embodiments above, in the compound of formula (III), R³³ is selected from halo and C₁₋₃ alkyl. In some embodiments in the compound of formula (III), R³³ is selected from F, Cl, Br and methyl.

In some embodiments in the compound of the formula (II) or (III), it may be that the group of the formula

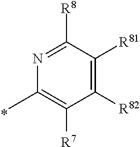

is selected from:

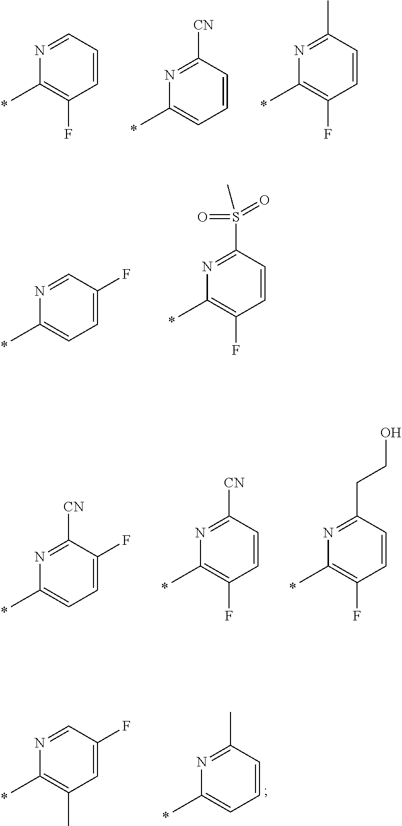

optionally in this embodiment it may also be that m and n are 0; R¹ is selected from H and halo (e.g F or Br). Suitably in this embodiment R⁴, R⁵ and R⁹ are H and R⁶ is H or methyl. Suitably in this embodiment R³³ in formula (III) is H or C₁₋₃ alkyl (e.g. R³³ H or methyl). Compounds of the formula (IV)

In some embodiments the compound of formula (I) is a compound of the formula (IV), or a pharmaceutically acceptable salt thereof as hereinbefore defined. In the compound of the formula (IV) R², R³, R⁴, R⁵, R⁶, R⁷, R⁸, R⁹, R⁸¹, R⁸², R³², R³³, m and n are as defined in relation to the compound of formula (I), or, unless stated otherwise, have any of the values defined herein including is one of more of (1) to (135) (in so far as those paragraphs are applicable to a compound of the formula (IV)). The following embodiments are directed to compounds of the formula (IV).

In some embodiments in the compound of formula (IV), R³² and R³³ are H; and n is 0.

In some embodiments in the compound of the formula (IV), it may be that R⁷ has any of the values in any one of paragraphs (65) to (74) above. It may be that R⁷ is selected from H, halo and C₁₋₃alkyl.

In some embodiments in the compound of the formula (IV), it may be that R⁸ has any of the values in any one of paragraphs (75) to (96) above. It may be that R⁸ is selected from H, —CN and —S(O)₂R¹⁰. It may be that R⁸ is C₁₋₃ alkyl.

In some embodiments, including the embodiments above, in the compound of formula (IV), R⁸¹ is selected from H, halo and C₁₋₃ alkyl; and R⁸² is H.

In some embodiments in the compound of formula (IV), R⁸¹ and R⁸² are H.

In some embodiments in the compound of formula (IV) R⁸¹ is selected from halo, C₁₋₄ alkyl and —CF₃; and R⁸² is H.

In some embodiments in the compound of formula (IV) R⁸² is selected from halo, C₁₋₄ alkyl and —CF₃; and R⁸² is H.

In some embodiments, including the embodiments above, in the compound of formula (IV) R⁸ is selected from H, —CN, C₁₋₄ alkyl, —S(O)₂R¹⁰ (e.g. —S(O)₂C₁₋₄ alkyl); and R⁷ selected from H, halo and C₁₋₃ alkyl. Preferably in this embodiment R⁷ and R⁸ are not both H.

In some embodiments, including the embodiments above, in the compound of formula (IV) R⁹ is H.

### Compounds of the Formulae (V) and (VI)

In some embodiments the compound of formula (I) is a compound of the formula (V) or formula (VI), or a pharmaceutically acceptable salt thereof as hereinbefore defined. In the compound of the formula (V) of (VI), R², R³, R⁴, R⁵, R⁶, R⁸, R⁹, R⁸¹, R⁸², X₁, X₂, X₃, m and n are as defined in relation to the compound of formula (I), or, unless stated otherwise, have any of the values defined herein including is one of more of (1) to (135) (in so far as those paragraphs are applicable to a compound of the formula (V) or formula (VI)). The following embodiments are directed to compounds of the formula (V) or formula (VI).

In some embodiments in the compound of formula (V) or formula (VI), one of R⁸¹ and R⁸² is H and the other is selected from: halo, C₁₋₄ alkyl, C₁₋₄ haloalkyl and —OC₁₋₄ alkyl.;

In some embodiments in the compound of formula (V) or formula (VI), one of R⁸¹ and R⁸² is H and the other is C₁₋₄ alkyl.

In some embodiments in the compound of formula (V) or formula (VI), one of R⁸¹ and R⁸² is H and the other is halo.

In some embodiments in the compound of formula (V) or formula (VI), R⁸¹ and R⁸² are both H.

In some embodiments in the compound of formula (V) or formula (VI), R⁸ is selected from any one of paragraphs (75) to (96) above. In some embodiments, including the embodiments above, in the compound of formula (V) or (VI), R⁸ is selected from: H, —CN, C₁₋₄ alkyl, —C(O)NH₂, —C(O)N(H)C₁₋₄ alkyl, —C(O)N(C₁₋₄ alkyl)₂, —S(O)₂C₁₋₄ alkyl, —C₁₋₄ alkyl-OH and —C₁₋₄ alkyl-OMe. It may be that R⁸ is selected from H, —CN and —S(O)₂R¹⁰.

In some embodiments in the compound of formula (V) or formula (VI), including any of the embodiments above, it may be that m is 0; X, is CR¹; and R¹ is selected from H, halo, —CN and C₁₋₃ alkyl.

In some embodiments in the compound of formula (V) or formula (VI), including any of the embodiments above, it may be that m is 0; n is 0; X, is CR¹; and R¹ is selected from H, halo and C₁₋₃ alkyl.

In some embodiments in the compound of formula (V) or formula (VI), including any of the embodiments above, it may be that m is 0; n is 0; X₂ is N; and X₃ is CR³³.

In some embodiments in the compound of formula (V) or formula (VI), including any of the embodiments above, it may be that m is 0 and X¹ is CH.

In some embodiments in the compound of formula (V) or formula (VI), including any of the embodiments above, it may be that R⁸¹ and R⁸² are H; and R⁸ is selected from: H, —CN, C₁₋₄ alkyl, —C₁₋₄ alkyl-OH, —C₁₋₄ alkyl and —S(O)₂C₁₋₄ alkyl.

In some embodiments in the compound of formula (V) or formula (VI), including any of the embodiments above, it may be that R⁹ is H.

Compounds of the formulae (VII) and (VIII)

In some embodiments the compound of formula (I) is a compound of the formula (VII) or formula (VIII), or a pharmaceutically acceptable salt thereof as hereinbefore defined. In the compound of the formula (VII) or formula (VIII) R², R³, R⁴, R⁵, R⁶, R¹, R⁹, R¹⁰, R⁸¹, R⁸², X₁, X₂, X₃, m and n are as defined in relation to the compound of formula (I), or, unless stated otherwise, have any of the values defined herein including is one of more of (1) to (135) (in so far as those paragraphs are applicable to a compound of the formula (VII) or formula (VIII)). The following embodiments are directed to compounds of the formula (VII) or formula (VIII).

In some embodiments in the compound of formula (VII) or formula (VIII) it may be that m is 0; X, is CR¹; and R¹ is selected from: H, halo, —CN and C₁₋₃ alkyl.

In some embodiments in the compound of formula (VII) or formula (VIII) it may be that m is 0; X, is CR¹; and R¹ is halo.

In some embodiments in the compound of formula (VII) or formula (VIII) it may be that m is 0; X, is CR¹; and R¹ is C₁₋₃ alkyl.

In some embodiments in the compound of formula (VII) or formula (VIII) it may be that m is 0; X, is CH.

In some embodiments in the compound of formula (VII) or formula (VIII), including any of the embodiments above, it may be that n is 0.

In some embodiments in the compound of formula (VII) or formula (VIII), including any of the embodiments above, it may be that n is 0; X₂ is N; and X₃ is CR³³.

In some embodiments in the compound of formula (VII) or formula (VIII), X₂ and X₃ are CH; and n is 0.

In some embodiments, including the embodiments above, in the compound of formula (VII) or formula (VIII), R⁸¹ is selected from H, halo and C₁₋₃ alkyl; and R⁸² is H.

In some embodiments in the compound of formula (VII) or formula (VIII), R⁸¹ and R⁸² are H.

In some embodiments in the compound of formula (VII) or formula (VIII) R⁸¹ is selected from halo, C₁₋₄ alkyl and —CF₃; and R⁸² is H.

In some embodiments in the compound of formula (VII) or formula (VIII) R⁸² is selected from halo, C₁-4 alkyl and —CF₃; and R⁸¹ is H.

In some embodiments in the compound of formula (VII) or formula (VIII), including any of the embodiments above, it may be that R⁷ has any of the values in any one of paragraphs (65) to (74) above. It may be that R⁷ is selected from H, halo and C₁₋₃alkyl. It may be that R⁷ is selected from fluoro and methyl. It may be that R⁷ is methyl.

In some embodiments in the compound of formula (VII) or formula (VIII), including any of the embodiments above, it may be that R⁸¹ and R⁸² are H; and R⁷ is selected from halo and C₁₋₃alkyl.

In some embodiments in the compound of formula (VII) or formula (VIII), including any of the embodiments above, it may be that R⁹ is H.

In some embodiments in the compound of formula (VIII), including any of the embodiments above, R¹⁰ is C₁₋₄ alkyl or C₃₋₆ cycloalkyl. For example, R¹⁰ is C₁₋₄ alkyl. It may be that R¹⁰ is methyl.

Compounds of the formula (IX)

In some embodiments the compound of formula (I) is a compound of the formula (IX), or a pharmaceutically acceptable salt thereof as hereinbefore defined. In the compound of the formula (IX) R², R³, R⁴, R⁵, R⁶, R⁷, R⁸, R⁹, X₁, X₂, X₃, m and n are as defined in relation to the compound of formula (I), or, unless stated otherwise, have any of the values defined herein including is one of more of (1) to (135) (in so far as those paragraphs are applicable to a compound of the formula (IX)). The following embodiments are directed to compounds of the formula (IX).

In some embodiments in the compound of formula (IX) it may be that m is 0; X₁ is CR¹; and R¹ is selected from: H, halo, —CN and C₁₋₃ alkyl.

In some embodiments in the compound of formula (IX) it may be that m is 0; X₁ is CR¹; and R¹ is halo.

In some embodiments in the compound of formula (IX) it may be that m is 0; X₁ is CR¹; and R¹ is C₁₋₃ alkyl.

In some embodiments in the compound of formula (IX) it may be that m is 0; X₁ is CH.

In some embodiments in the compound of formula (IX), including any of the embodiments above, it may be that n is 0.

In some embodiments in the compound of formula (VII) or formula (VIII), including any of the embodiments above, it may be that n is 0; X₂ is N; and X₃ is CR³³.

In some embodiments in the compound of formula (VII) or formula (VIII), X₂ and X₃ are CH; and n is 0.

In some embodiments in the compound of the formula (IX), including any of the embodiments above, it may be that R⁷ has any of the values in any one of paragraphs (65) to (74) above. It may be that R⁷ is selected from H and C₁₋₃ alkyl.

In some embodiments in the compound of the formula (IX), including any of the embodiments above, it may be that R⁸ has any of the values in any one of paragraphs (75) to (96) above. It may be that R⁸ is selected from H, —CN and —S(O)₂R¹⁰.

In some embodiments in the compound of the formula (IX), including any of the embodiments above it may be that R⁸ selected from H, —CN, C₁₋₄ alkyl, —C₁₋₄ alkyl-OH, —C₁₋₄ alkyl-OMe, —O—C₂₋₄ alkyl-OH, —O—C₂₋₄ alkyl-OMe, —C(O)NH₂, —C(O)N(H)C₁₋₃alkyl, —C(O)N(C₁₋₃ alkyl)₂ and —S(O)₂C₁₋₄ alkyl).

In some embodiments in the compound of the formula (IX), including any of the embodiments above it may be that R⁸ selected from H, —CN and C₁₋₄ alkyl; and R⁷ is selected from H and C₁₋₃ alkyl.

In some embodiments in the compound of the formula (IX), including any of the embodiments above it may be that R⁹ is H.

In some embodiments in the compounds of formulae (I), (II), (III) and (IV) including any of the embodiments above, it may be that the group of the formula:

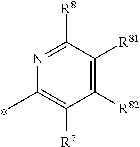

is selected from:

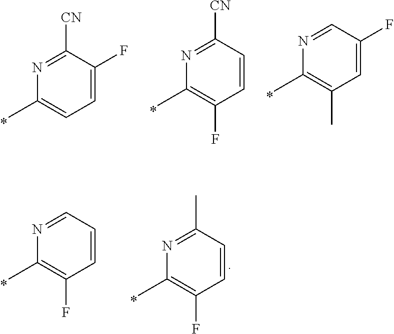

In some embodiments in the compounds of formulae (I), (II), (III) and (IV), including any of the embodiments above, it may be that the group of the formula:

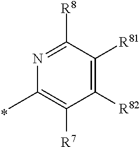

is selected from:

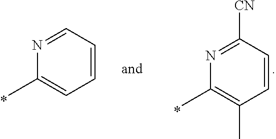

In some embodiments the compound of the formula (I) is a compound of the formula (IIIa), or a pharmaceutically acceptable salt thereof:

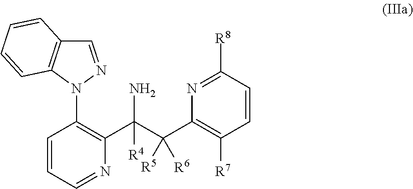

wherein R⁷ is selected from H, fluoro and C₁₋₃ alkyl; and R⁸ is H or —CN. In this embodiment it may be that R⁷ is selected from H, F and methyl, and R⁸ is —CN. In this embodiment it may be that R⁷ is methyl and R⁸ is —CN. In this embodiment it may be that R⁷ and R⁸ are both H.

In some embodiments in the compounds of formulae (I), (II), (III), (IIIa), (IV), (V), (VI), (VII), (VIII) and (IX) including any of the embodiments above, it may be that R⁴ and R⁵ are H and R⁶ is selected from: H and methyl.

In some embodiments in the compounds of formulae (I), (II), (III), (IIIa), (IV), (V), (VI), (VII), (VIII) and (IX) including any of the embodiments above, it may be that the group

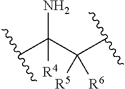

is of the formula

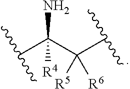

In some embodiments in the compounds of formulae (I), (II), (III), (IIIa), (IV), (V), (VI), (VII), (VIII) and (IX) including any of the embodiments above, it may be that the group

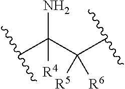

is of the formula

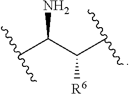

In some embodiments in the compounds of formulae (I), (II), (III), (IIIa), (IV), (V), (VI), (VII), (VIII) and (IX) including any of the embodiments above, it may be that the group

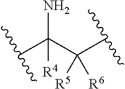

is of the formula

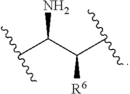

In some embodiments in the compounds of formulae (I), (II), (III), (IIIa), (IV), (V), (VI), (VII), (VIII) and (IX) including any of the embodiments above, it may be that the group

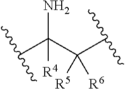

is:

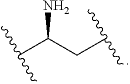 [SMILES: *C[C@@H](*)N]

In another embodiment there is provided a compound selected from any one of the Examples herein, or a pharmaceutically acceptable salt or prodrug thereof.

In another embodiment there is provided a compound selected from Table 1, or a pharmaceutically acceptable salt or prodrug thereof. In particular there is provided a compound selected from Table 1, or a pharmaceutically acceptable salt thereof:

TABLE 1

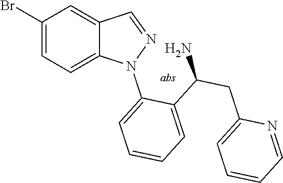 [SMILES: N[C@@H](Cc1ccccn1)c1ccccc1-n1ncc2cc(Br)ccc21]

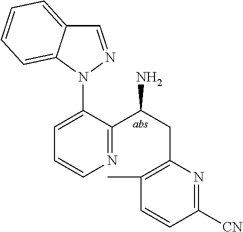 [SMILES: Cc1ccc(C#N)nc1C[C@H](N)c1ncccc1-n1ncc2ccccc21]

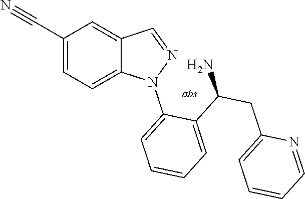 [SMILES: N#Cc1ccc2c(cnn2-c2ccccc2[C@@H](N)Cc2ccccn2)c1]

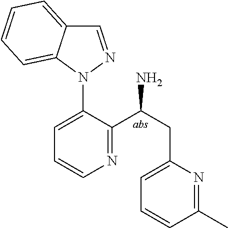 [SMILES: Cc1cccc(C[C@H](N)c2ncccc2-n2ncc3ccccc32)n1]

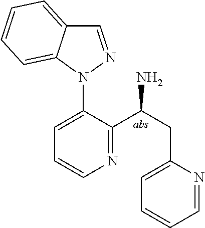 [SMILES: N[C@@H](Cc1ccccn1)c1ncccc1-n1ncc2ccccc21]

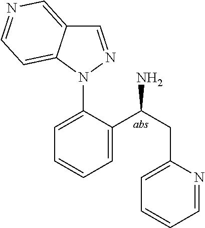 [SMILES: N[C@@H](Cc1ccccn1)c1ccccc1-n1ncc2cnccc21]

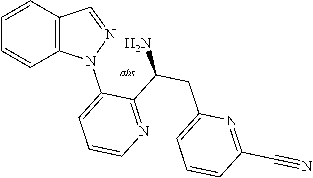 [SMILES: N#Cc1cccc(C[C@H](N)c2ncccc2-n2ncc3ccccc32)n1]

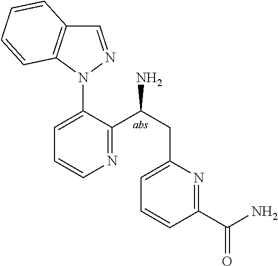 [SMILES: NC(=O)c1cccc(C[C@H](N)c2ncccc2-n2ncc3ccccc32)n1]

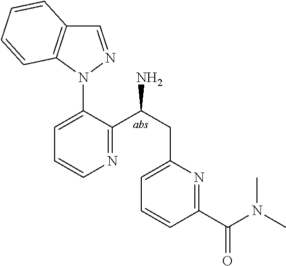 [SMILES: CN(C)C(=O)c1cccc(C[C@H](N)c2ncccc2-n2ncc3ccccc32)n1]

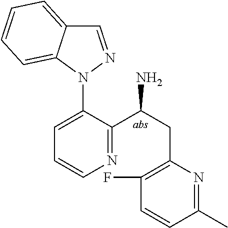 [SMILES: Cc1ccc(F)c(C[C@H](N)c2ncccc2-n2ncc3ccccc32)n1]

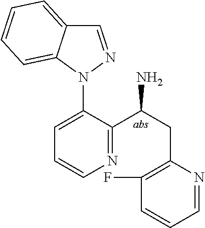 [SMILES: N[C@@H](Cc1ncccc1F)c1ncccc1-n1ncc2ccccc21]

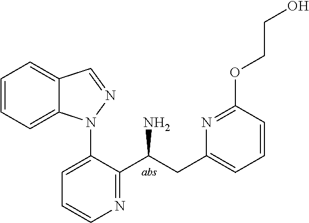 [SMILES: N[C@@H](Cc1cccc(OCCO)n1)c1ncccc1-n1ncc2ccccc21]

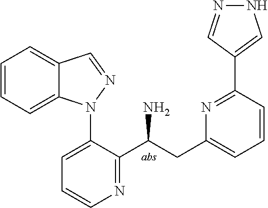 [SMILES: N[C@@H](Cc1cccc(-c2cn[nH]c2)n1)c1ncccc1-n1ncc2ccccc21]

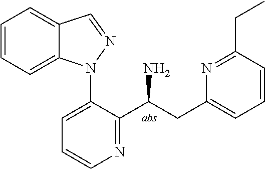 [SMILES: CCc1cccc(C[C@H](N)c2ncccc2-n2ncc3ccccc32)n1]

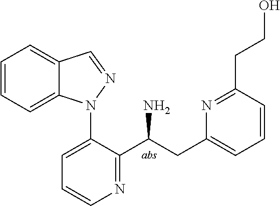 [SMILES: N[C@@H](Cc1cccc(CCO)n1)c1ncccc1-n1ncc2ccccc21]

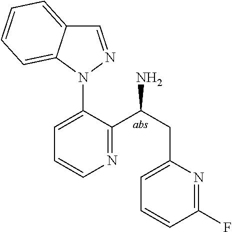 [SMILES: N[C@@H](Cc1cccc(F)n1)c1ncccc1-n1ncc2ccccc21]

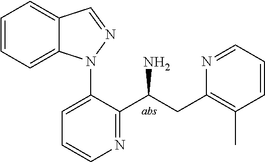 [SMILES: Cc1cccnc1C[C@H](N)c1ncccc1-n1ncc2ccccc21]

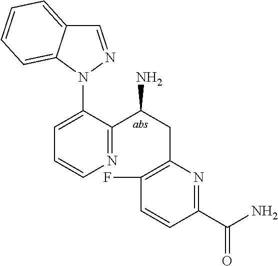 [SMILES: NC(=O)c1ccc(F)c(C[C@H](N)c2ncccc2-n2ncc3ccccc32)n1]

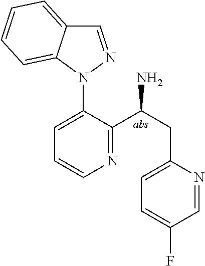 [SMILES: N[C@@H](Cc1ccc(F)cn1)c1ncccc1-n1ncc2ccccc21]

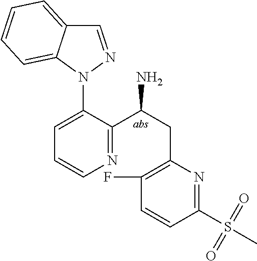 [SMILES: CS(=O)(=O)c1ccc(F)c(C[C@H](N)c2ncccc2-n2ncc3ccccc32)n1]

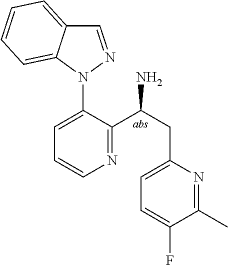 [SMILES: Cc1nc(C[C@H](N)c2ncccc2-n2ncc3ccccc32)ccc1F]

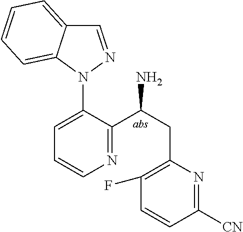 [SMILES: N#Cc1ccc(F)c(C[C@H](N)c2ncccc2-n2ncc3ccccc32)n1]

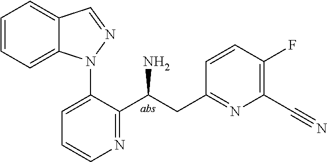 [SMILES: N#Cc1nc(C[C@H](N)c2ncccc2-n2ncc3ccccc32)ccc1F]

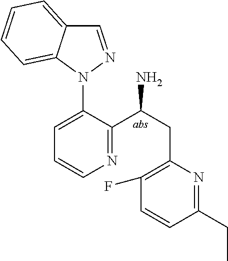 [SMILES: CCc1ccc(F)c(C[C@H](N)c2ncccc2-n2ncc3ccccc32)n1]

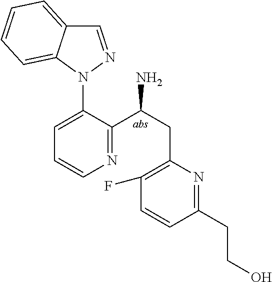 [SMILES: N[C@@H](Cc1nc(CCO)ccc1F)c1ncccc1-n1ncc2ccccc21]

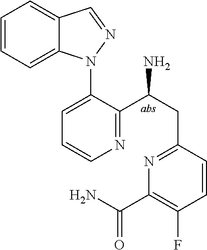 [SMILES: NC(=O)c1nc(C[C@H](N)c2ncccc2-n2ncc3ccccc32)ccc1F]

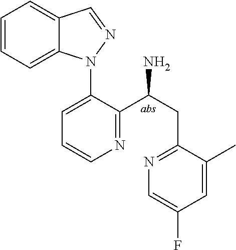 [SMILES: Cc1cc(F)cnc1C[C@H](N)c1ncccc1-n1ncc2ccccc21]

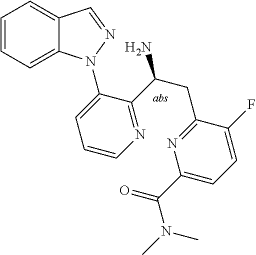 [SMILES: CN(C)C(=O)c1ccc(F)c(C[C@H](N)c2ncccc2-n2ncc3ccccc32)n1]

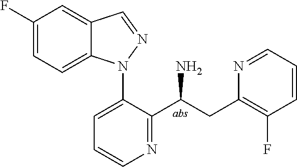 [SMILES: N[C@@H](Cc1ncccc1F)c1ncccc1-n1ncc2cc(F)ccc21]

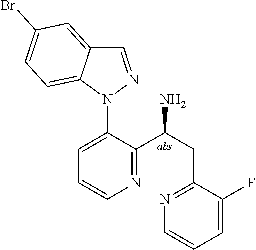 [SMILES: N[C@@H](Cc1ncccc1F)c1ncccc1-n1ncc2cc(Br)ccc21]

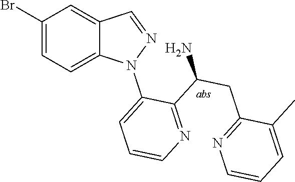 [SMILES: Cc1cccnc1C[C@H](N)c1ncccc1-n1ncc2cc(Br)ccc21]

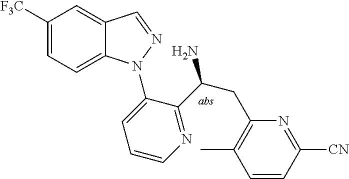 [SMILES: Cc1ccc(C#N)nc1C[C@H](N)c1ncccc1-n1ncc2cc(C(F)(F)F)ccc21]

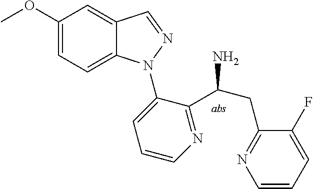 [SMILES: COc1ccc2c(cnn2-c2cccnc2[C@@H](N)Cc2ncccc2F)c1]

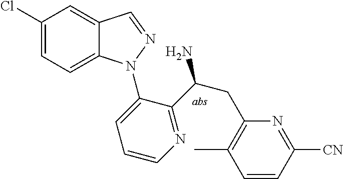 [SMILES: Cc1ccc(C#N)nc1C[C@H](N)c1ncccc1-n1ncc2cc(Cl)ccc21]

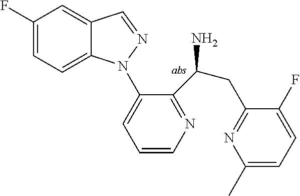 [SMILES: Cc1ccc(F)c(C[C@H](N)c2ncccc2-n2ncc3cc(F)ccc32)n1]

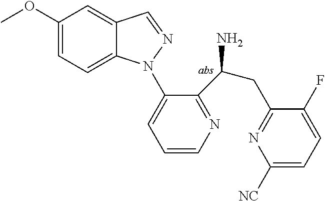 [SMILES: COc1ccc2c(cnn2-c2cccnc2[C@@H](N)Cc2nc(C#N)ccc2F)c1]

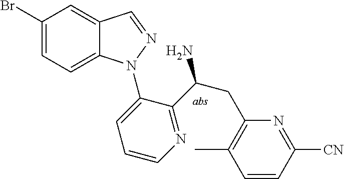 [SMILES: Cc1ccc(C#N)nc1C[C@H](N)c1ncccc1-n1ncc2cc(Br)ccc21]

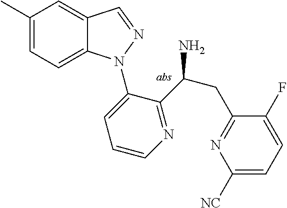 [SMILES: Cc1ccc2c(cnn2-c2cccnc2[C@@H](N)Cc2nc(C#N)ccc2F)c1]

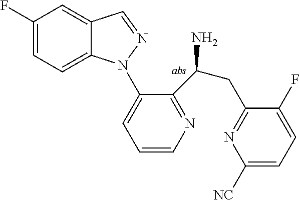 [SMILES: N#Cc1ccc(F)c(C[C@H](N)c2ncccc2-n2ncc3cc(F)ccc32)n1]

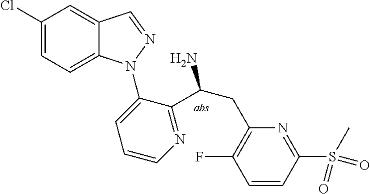 [SMILES: CS(=O)(=O)c1ccc(F)c(C[C@H](N)c2ncccc2-n2ncc3cc(Cl)ccc32)n1]

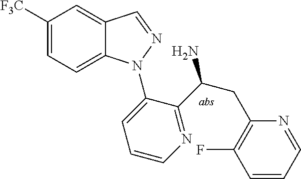 [SMILES: N[C@@H](Cc1ncccc1F)c1ncccc1-n1ncc2cc(C(F)(F)F)ccc21]

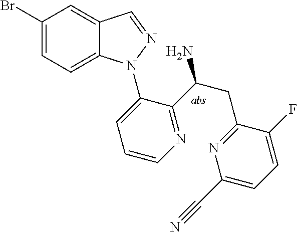 [SMILES: N#Cc1ccc(F)c(C[C@H](N)c2ncccc2-n2ncc3cc(Br)ccc32)n1]

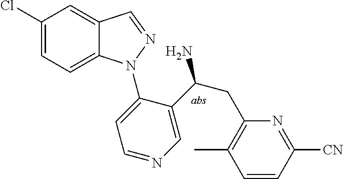 [SMILES: Cc1ccc(C#N)nc1C[C@H](N)c1cnccc1-n1ncc2cc(Cl)ccc21]

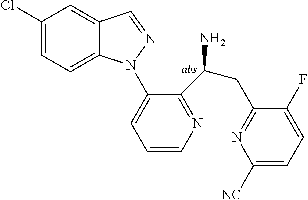 [SMILES: N#Cc1ccc(F)c(C[C@H](N)c2ncccc2-n2ncc3cc(Cl)ccc32)n1]

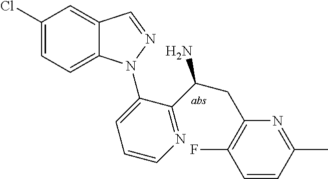 [SMILES: Cc1ccc(F)c(C[C@H](N)c2ncccc2-n2ncc3cc(Cl)ccc32)n1]

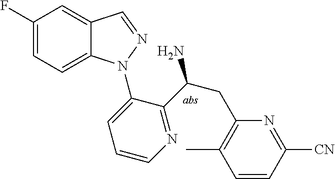 [SMILES: Cc1ccc(C#N)nc1C[C@H](N)c1ncccc1-n1ncc2cc(F)ccc21]

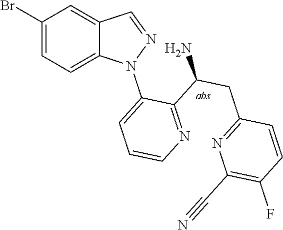 [SMILES: N#Cc1nc(C[C@H](N)c2ncccc2-n2ncc3cc(Br)ccc32)ccc1F]

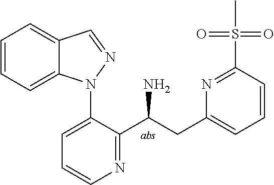 [SMILES: CS(=O)(=O)c1cccc(C[C@H](N)c2ncccc2-n2ncc3ccccc32)n1]

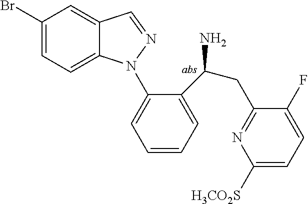

In another embodiment there is provided a compound selected from Table 2, or a pharmaceutically acceptable salt or prodrug thereof. In particular there is provided a compound selected from Table 2, or a pharmaceutically acceptable salt thereof:

TABLE 2

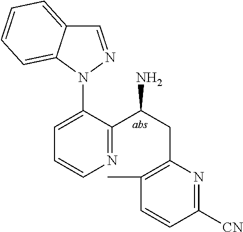 [SMILES: Cc1ccc(C#N)nc1C[C@H](N)c1ncccc1-n1ncc2ccccc21]

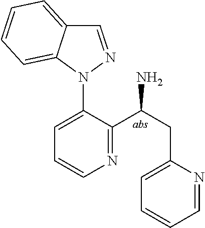 [SMILES: N[C@@H](Cc1ccccn1)c1ncccc1-n1ncc2ccccc21]

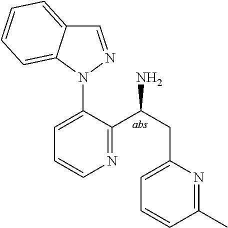 [SMILES: Cc1cccc(C[C@H](N)c2ncccc2-n2ncc3ccccc32)n1]

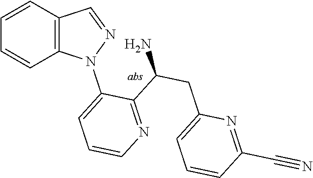 [SMILES: N#Cc1cccc(C[C@H](N)c2ncccc2-n2ncc3ccccc32)n1]

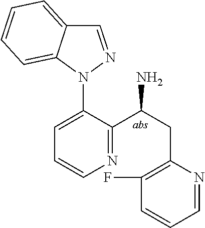 [SMILES: N[C@@H](Cc1ncccc1F)c1ncccc1-n1ncc2ccccc21]

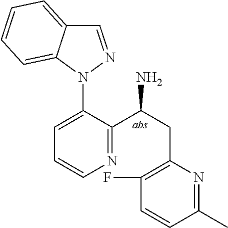 [SMILES: Cc1ccc(F)c(C[C@H](N)c2ncccc2-n2ncc3ccccc32)n1]

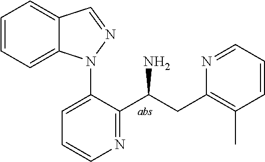 [SMILES: Cc1cccnc1C[C@H](N)c1ncccc1-n1ncc2ccccc21]

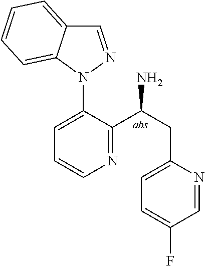 [SMILES: N[C@@H](Cc1ccc(F)cn1)c1ncccc1-n1ncc2ccccc21]

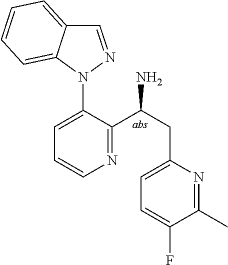 [SMILES: Cc1nc(C[C@H](N)c2ncccc2-n2ncc3ccccc32)ccc1F]

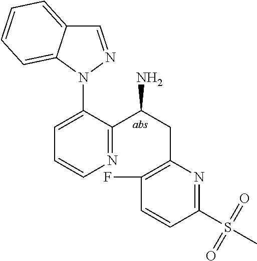 [SMILES: CS(=O)(=O)c1ccc(F)c(C[C@H](N)c2ncccc2-n2ncc3ccccc32)n1]

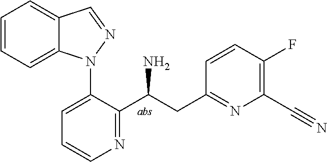 [SMILES: N#Cc1nc(C[C@H](N)c2ncccc2-n2ncc3ccccc32)ccc1F]

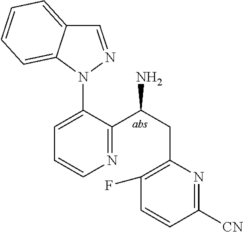 [SMILES: N#Cc1ccc(F)c(C[C@H](N)c2ncccc2-n2ncc3ccccc32)n1]

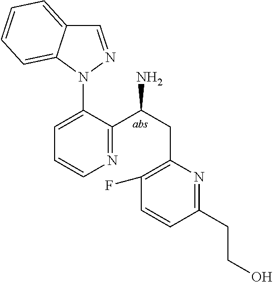 [SMILES: N[C@@H](Cc1nc(CCO)ccc1F)c1ncccc1-n1ncc2ccccc21]

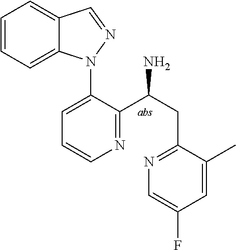 [SMILES: Cc1cc(F)cnc1C[C@H](N)c1ncccc1-n1ncc2ccccc21]

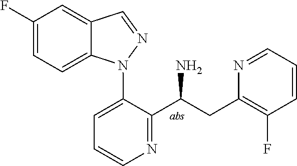 [SMILES: N[C@@H](Cc1ncccc1F)c1ncccc1-n1ncc2cc(F)ccc21]

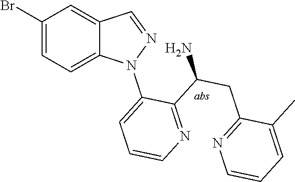 [SMILES: Cc1cccnc1C[C@H](N)c1ncccc1-n1ncc2cc(Br)ccc21]

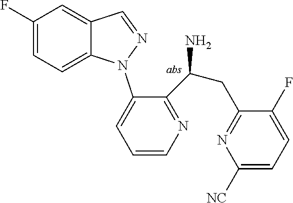 [SMILES: N#Cc1ccc(F)c(C[C@H](N)c2ncccc2-n2ncc3cc(F)ccc32)n1]

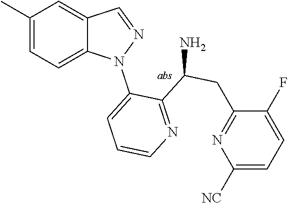 [SMILES: Cc1ccc2c(cnn2-c2cccnc2[C@@H](N)Cc2nc(C#N)ccc2F)c1]

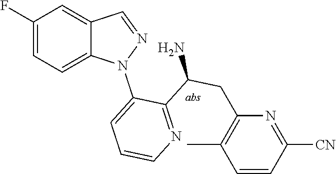 [SMILES: Cc1ccc(C#N)nc1C[C@H](N)c1ncccc1-n1ncc2cc(F)ccc21]

### Pharmaceutical Compositions

In accordance with another aspect, the present invention provides a pharmaceutical composition comprising a compound of the invention or a pharmaceutically acceptable salt thereof, and a pharmaceutically acceptable excipient.

Conventional procedures for the selection and preparation of suitable pharmaceutical compositions are described in, for example, “Pharmaceuticals—The Science of Dosage Form Designs”, M. E. Aulton, Churchill Livingstone, 1988.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intraperitoneal dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy of a condition is an amount sufficient to symptomatically relieve in a warm-blooded animal, particularly a human the symptoms of the condition or to slow the progression of the condition.

The amount of active ingredient that is combined with one or more excipients to produce a single dosage form will necessarily vary depending upon the host treated and the particular route of administration. For example, a formulation intended for oral administration to humans will generally contain, for example, from 0.1 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the invention will naturally vary according to the nature and severity of the conditions, the age and sex of the animal or patient and the route of administration, according to well-known principles of medicine.

In using a compound of the invention for therapeutic or prophylactic purposes it will generally be administered so that a daily dose in the range, for example, a daily dose selected from 0.05 mg/kg to 100 mg/kg, 0.1 mg/kg to 100 mg/kg, 1 mg/kg to 75 mg/kg, 1 mg/kg to 50 mg/kg, 1 mg/kg to 20 mg/kg, 5 mg/kg to 10 mg/kg, 0.1 mg/kg to 5 mg/kg, 0.1 mg/kg to 2 mg/kg or 0.1 mg/kg to 1 mg/kg body weight is received, given if required in divided doses. In general lower doses will be administered when a parenteral route is employed. Thus, for example, for intravenous, subcutaneous, intramuscular or intraperitoneal administration, a dose in the range, for example, 0.05 mg/kg to 30 mg/kg, 0.1 mg/kg to 30 mg/kg, 0.1 mg/kg to 5 mg/kg, 0.1 mg/kg to 2 mg/kg or 0.1 mg/kg to 1 mg/kg body weight will generally be used. Similarly, for administration by inhalation, a dose in the range, for example, 0.05 mg/kg to 25 mg/kg body weight will be used. Suitably the compound of the invention is administered orally, for example in the form of a tablet, or capsule dosage form. The daily dose administered orally may be, for example a total daily dose selected from 1 mg to 1000 mg, 5 mg to 1000 mg, 10 mg to 750 mg, 25 mg to 500 mg, 1 mg to 100 mg, 5 mg to 75 mg, or 10 mg to 50 mg. Typically, unit dosage forms will contain about 0.5 mg to 0.5 g of a compound of this invention. In a particular embodiment the compound of the invention is administered parenterally, for example by intravenous administration. In another particular embodiment the compound of the invention is administered orally.

The compounds of the invention may be administered at a dosage interval of, for example once every hour, once every 2 hours, once every 4 hours, once every 6 hours, once every 8 hours, or once every 12 hours. In some embodiments the compound is administered once per day, twice per day, three times per day, four times per day, once every 2 days, or once per week. Suitably the compound of the invention is administered once or twice per day.

Regular dosing of the compound of the invention may provide a cumulative, and sustained analgesic effect. The Examples herein show that a single injection of a compound of the invention results in analgesia, but the analgesic effect reduces towards the baseline level within a few hours of administration. Regular repeated dosing of a compound of the invention may provide a cumulative and sustained analgesic effect. The cumulative effect on analgesia provided by the compounds of the invention may enable the compound to be administered at a dose which is lower than the dose required to give a full analgesic effect administered as a single bolus dose. Accordingly, regular administration of a low dose of a compound of the invention may provide a greater therapeutic window between analgesia and undesirable side-effects which might be associated with higher doses, for example bradycardia or tremors.

In certain embodiments a compound of the invention is administered regularly so as to provide a plasma concentration of 10% to 120% of the analgesic ED₅₀ for the compound. For example the compound may be administered at a dose which provides from 10% to 100%, from 10% to 80%, from 10% to 60%, from 15% to 50%, from 20% to 50%, from 25% to 50% or from 25% to 45% of the analgesic ED₅₀ of the compound. The regular dosage interval may be, for example, any of the dosage intervals set out above.

### Therapeutic Uses and Applications

In accordance with another aspect, the present invention provides a compound of the invention, or a pharmaceutically acceptable salt thereof, or a pharmaceutical composition of the invention, for use as a medicament.

A further aspect of the invention provides a compound of the invention, or a pharmaceutically acceptable salt thereof, or a pharmaceutical composition of the invention, for use in the treatment of a disease or medical condition mediated by hyperpolarisation activated cyclic-nucleotide modulated ion channel 2 (HCN2).

Also provided is the use of a compound of the invention, or a pharmaceutically acceptable salt thereof, or a pharmaceutical composition of the invention, in the manufacture of a medicament for the treatment of a disease or medical condition mediated by HCN2.

Also provided is a method of treating a disease or medical condition mediated by HCN2 in a subject in need thereof, the method comprising administering to the subject an effective amount of: (i) a compound of the invention, or a pharmaceutically acceptable salt thereof; or (ii) a pharmaceutical composition of the invention.

The conditions mediated by HCN2 may be, for example, any of the conditions disclosed herein.

The compounds of the invention are HCN2 inhibitors, useful in the treatment of a conditions in which inhibition of HCN2 ion channels is beneficial. As discussed in the Background to the Invention, the disclosure of which is incorporated into the main description, HCN4 is highly expressed in cardiac tissue and is the major regulator of cardiac pacemaking. Inhibition of HCN4 induces bradycardia and deletion of HCN4 in mice, either globally, or locally in the heart, is lethal. Accordingly, compounds which significantly inhibit HCN4 in addition to HCN2 would not be suitable as a chronic treatment, for example as an analgesic used for the chronic treatment of pain. Preferred compounds of the invention selectively inhibit HCN2 over HCN4. HCN2 selective compounds are expected to reduce or eliminate the risks of undesirable cardiac side-effects associated with the use of a compound of the invention as a medicament for the treatment of conditions mediated by HCN2. In preferred embodiments a compound of the invention exhibits an IC₅₀ in the HCN2 assay described herein (see Example 50) which is at least 2 times, for example at least 5 times, at least 10 times, at least 20 times or at least 30 times lower than the IC₅₀ of the same compound measured in the HCN4 assay described herein (see Example 50).

HCN1 channels are also expressed in cardiac tissue and are associated with cardiac function. Accordingly, preferred compounds of the invention selectively inhibit HCN2 over HCN1. In some embodiments a compound of the invention exhibits an IC₅₀ in the HCN2 assay described herein (see Example 50) which is at least 2 times, for example at least 5 times, at least 10 times or at least 20 times lower than the IC₅₀ of the same compound measured in the HCN1 assay described herein (see Example 50).

When comparing the IC₅₀ values for a compound in an HCN assay to assess the selectivity of the compound, the same assay protocol should be used to generate the IC₅₀ values for the compound. For example, both IC₅₀ values should be measured using the PatchXpress protocol set out in Example 50A or both should be measured using the Sophion Qube protocol set out in Example 50B.

The voltage-gated Na⁺ channel Naᵥ1.5 is found predominantly in cardiac muscle. It initiates the cardiac action potential in the heart and is essential for conduction of the electrical impulse, as well as the action potential duration. In preferred embodiments a compound of the invention selectively inhibits HCN2 over Naᵥ1.5. In some embodiments a compound of the invention exhibits an IC₅₀ in the HCN2 assay described herein (see Example 50) which is at least 2 times, for example at least 5 times, at least 10 times, at least 20 times or at least 50 times lower than the IC₅₀ of the same compound measured in the Naᵥ1.5 assay described herein (see Example 52). Suitably the IC₅₀ for HCN2 is measured using the Sophion Qube protocol set out in Example 50B and the IC₅₀ value for Naᵥ1.5 is determined using the Sophion Qube protocol set out in Example 52B.

It is well known that drugs which inhibit the hERG potassium channel in the heart can result in delayed ventricular repolarization (QT interval prolongation). Preferred compounds of the invention are those with a low hERG liability. In some embodiments a compound of the invention exhibits an IC₅₀ in the HCN2 assay described herein which is at least 2 times, for example at least 5 times, at least 10 times or at least 20 times lower than the IC₅₀ of the same compound measured in a hERG assay described herein (see Example 51). Suitably the IC₅₀ for HCN2 and for hERG are measured using the Sophion Qube protocols set out in Example 50B and 51B respectively.

Accordingly, in preferred embodiments a compound of the invention has a high therapeutic window between the concentration required for inhibition of HCN2 and ion channels associated with cardiac function. In some embodiments compounds of the invention are selective for HCN2 over one or more of HCN4, HCN1, Naᵥ1.5 or hERG. In particular embodiments preferred compounds of the invention selectively inhibit HCN2 over HCN4 and/or HNC1. In further particular embodiments preferred compounds of the invention selectively inhibit HCN2 over HCN4.

HCN2 channels are widely expressed in the brain and significant inhibition of HCN2 in the brain could induce undesirable CNS side-effects such as tremors or ataxia. In preferred embodiments, compounds of the invention are peripherally restricted HCN2 inhibitors such that when present at therapeutically effective concentrations in peripheral tissues, only low levels of the compound are present in the brain at a concentration below that necessary to induce undesirable CNS associated side effects. In some embodiments the compound of the invention is a substrate for the transporter β-glycoprotein (P-gp). P-gp substrates are generally effluxed at the brain endothelium. Accordingly, compounds which are P-gp substrates are expected to exhibit low concentrations in brain tissue. In some embodiments a compound of the invention has a high efflux ratio when measured in the MDCK-MDR1 permeability assay described herein (see Example 53). The MDCK-MDR1 assay described in Example 53 run in the absence and presence of a P-gp inhibitor can be used to identify compounds having the potential to be peripherally restricted. A net flux value >5 (i.e. efflux ratio without inhibitor divided by efflux ratio plus inhibitor) is indicative of compounds being substrates for the transporter P-gp and would therefore have a greater likelihood of being restricted from the CNS (i.e. compounds with low CNS penetration). In some embodiments a compound of the invention with low CNS penetration has a net flux of or more, for example 10 or more, 15 or more, or 20 or more when measured in the MDCK-MDR1 permeability assay described herein. Compounds of the invention which exhibit low CNS penetration following administration, are referred to herein as “peripherally restricted compounds” or “peripherally restricted HCN2 inhibitors”.

In the following sections of the application reference is made to a compound of the invention, or a pharmaceutically acceptable salt thereof for use in the treatment of certain diseases or conditions. It is to be understood that any reference herein to a compound for a particular use is also intended to be a reference to (i) the use of the compound of the invention, or pharmaceutically acceptable salt thereof, in the manufacture of a medicament for the treatment of that disease or condition; and (ii) a method of treating the disease or condition in a subject, the method comprising administering to the subject a therapeutically effective amount of the compound of the invention, or pharmaceutically acceptable salt thereof.

The disease or medical condition mediated by HCN2 may be any of the diseases or medical conditions listed in this application.

### Pain

In some embodiments a compound of the invention is for use in the treatment or prevention of pain generally, including, but not limited to NP and IP.

### Neuropathic Pain

In some embodiments a compound of the invention is for use in the treatment or prevention of neuropathic pain. In some embodiments a compound of the invention is for use in the treatment or prevention of peripheral neuropathic pain. Examples of NP include, but are not limited to neuropathic pain selected from painful diabetic neuropathy (PDN), post-herpetic neuralgia (PHN), pain associated with cancer, chemotherapy induced pain including, chemotherapy-induced peripheral neuropathy, post-operative pain (e.g. post-mastectomy syndrome, post-thoracotomy syndrome or phantom pain), trigeminal neuralgia, complex regional pain syndrome (CRPS), opioid resistant pain, pudendal neuralgia and neuropathic pain associated with lower back pain, nerve damage following traumatic injury (e.g. whiplash injury in car crash) and carpal tunnel syndrome.

In some embodiments a compound of the invention is for use in the treatment or prevention of neuropathic pain associated with or resulting from: neurological disorders, spine and peripheral nerve surgery, spinal cord trauma, chronic pain syndrome, fibromyalgia, chronic fatigue syndrome, neuralgias (e.g. trigeminal neuralgia, glossopharyngeal neuralgia, postherpetic neuralgia and causalgia), lupus, HIV infection, sarcoidosis, peripheral neuropathy, bilateral peripheral neuropathy, diabetic neuropathy, sciatic neuritis, mandibular joint neuralgia, peripheral neuritis, polyneuritis, stump pain, phantom limb pain, bony fractures, oral neuropathic pain, Charcot's pain, complex regional pain syndrome I and II (CRPS VIT), radiculopathy, Guillain-Barre syndrome, meralgia paresthetica, burning-mouth syndrome, optic neuritis, postfebrile neuritis, migrating neuritis, segmental neuritis, Gombault's neuritis, neuronitis, cervicobrachial neuralgia, cranial neuralgia, geniculate neuralgia, glossopharyngial neuralgia, idiopathic neuralgia, intercostals neuralgia, mammary neuralgia, Morton's neuralgia, nasociliary neuralgia, occipital neuralgia, red neuralgia, Sluder's neuralgia, splenopalatine neuralgia, supraorbital neuralgia, vulvodynia, or vidian neuralgia. In one embodiment the compound of the invention is for use in the treatment of postherpetic neuralgia.

In some embodiments a compound of the invention is for use in the prevention or relief of one or more of the symptoms of NP, for example dysesthesia (spontaneous or evoked burning pain, often with a superimposed lancinating component), deep pain, aching pain, hyperesthesia, hyperalgesia, allodynia and hyperpathia.

Preferred compounds are those which treat neuropathic pain (particularly peripheral neuropathic pain) whilst maintaining the perception of acute pain.

### Inflammatory Pain

In some embodiments a compound of the invention is for use in the treatment or prevention of inflammatory pain. In some embodiments the pain is chronic inflammatory pain. In some embodiments the pain is acute inflammatory pain. In some embodiments a compound of the invention is for use in the treatment or prevention of inflammatory pain, especially chronic inflammatory pain, resulting from or associated with one or more of: inflammatory bowel disease, visceral pain, post-operative pain, osteoarthritis, rheumatoid arthritis, back pain, lower back pain, joint pain, abdominal pain, chest pain, labour, musculoskeletal diseases, skin diseases, toothache, pyresis, burn, sunburn, animal or insect bite or sting, neurogenic bladder, interstitial cystitis, urinary tract infection, rhinitis, dermatitis including contact dermatitis and atopic dermatitis, pharyngitis, mucositis, enteritis, irritable bowel syndrome, cholecystitis, pancreatitis, postmastectomy pain syndrome, menstrual pain, endometriosis, sinus headache, tension headache, or arachnoiditis.

In some embodiments a compound of the invention is for use in the treatment of inflammatory hyperalgesia, including inflammatory somatic hyperalgesia or inflammatory visceral hyperalgesia. Inflammatory somatic hyperalgesia can be characterized by the presence of an inflammatory hyperalgesic state in which a hypersensitivity to thermal, mechanical and/or chemical stimuli exists. Inflammatory visceral hyperalgesia can also be characterized by the presence of an inflammatory hyperalgesic state, in which an enhanced visceral irritability exists.

Emery et al 2011, 2012, ibid, have shown that the selective deletion of HCN2 in the NaV1.8-expressing sensory neurones in mice showed that the perception of pain resulting from inflammatory stimuli was lost, however, reaction to acute pain in the absence of inflammatory stimuli was maintained. Thus preferred compounds are those which treat inflammatory pain whilst maintaining the perception of acute pain.

### Tinnitus

As set out in the Background to the Invention, and illustrated in the Examples, the inventors have for the first time shown that tinnitus can be treated using an HCN2 inhibitor in animal models. The Examples suggest that the effects observed are applicable to any HCN2 inhibitor and are not limited to a compound of the invention.

In one embodiment of the invention there is provided an HCN2 inhibitor for use in the treatment of tinnitus or a related condition. In a preferred embodiment the HCN2 inhibitor is a compound of the invention. Accordingly there is provided a compound of the invention, for use in the prevention or treatment of tinnitus or a related condition.

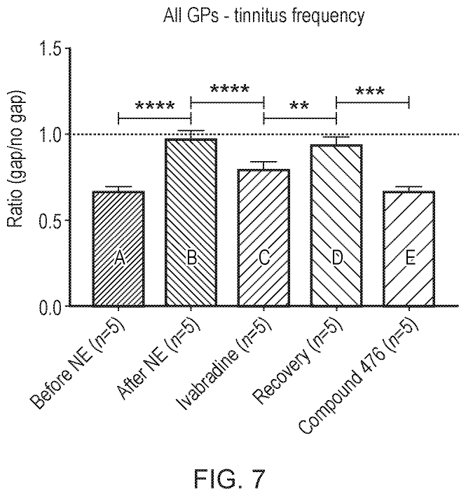

Ivabradine is a peripherally restricted compound, with pan-HCN inhibitory action. The Examples herein show that despite being peripherally restricted the compound successfully treated tinnitus. Similar results were obtained using a peripherally restrictive and selective HCN2 inhibitor compound (Compound 476 in FIG. 7). The experiments therefore suggest that tinnitus may be treated without the need for CNS penetration, thereby avoiding undesirable side effects that might be associated with HCN2 inhibition in the CNS such as tremors or ataxia.

Accordingly, also provided is a peripherally restricted HCN2 inhibitor for use in the treatment of tinnitus or a related condition. In some embodiments the peripherally restricted HCN2 inhibitor is a peripherally restricted HCN2 inhibitor, for example ivabradine. In preferred embodiments the peripherally restricted HCN2 inhibitor is peripherally restricted compound of the invention.

Tinnitus may occur as objective tinnitus, or subjective tinnitus. Subjective tinnitus is the most common type of tinnitus. Subjective tinnitus, also known as sensorineural tinnitus can only be heard by the affected person. Objective tinnitus, on the other hand, can be detected by other people and is usually caused by myoclonus or a vascular condition, although in some cases, tinnitus is generated by a self-sustained oscillation within the ear. In preferred embodiments the HCN2 inhibitor (preferably a compound of the invention) is for use in the treatment of subjective tinnitus. The tinnitus may be acute tinnitus, however, in preferred embodiments the tinnitus is chronic tinnitus, for example tinnitus that persists for more than 2 weeks, more than 1 month or more than 6 months.

In some embodiments the HCN2 inhibitor (preferably a compound of the invention) is for use in the treatment or prevention of tinnitus caused by or associated with one of more of: exposure to loud noise; presbyacusis (hearing loss); ear or head injuries, ear infections; tumours which impact on auditory nerves; Meniere's disease; cardiovascular disease, cerebrovascular disease; hyperthyroidism; hypothyroidism; side-effects of a drug therapy (for example salicylates (including mesalamine or aspirin), particularly when taken in high doses), quinine anti-malarial agents, aminoglycoside antibiotics, chemotherapy (including, but not limited to platinum cytotoxic agents (e.g. cisplatin, carboplatin and oxaliplatin)) or loop diuretics (e.g. furosemide, ethacrynic acid and torsemide); or an auditory dysfunction (e.g. hyperacusis, distortion of sounds, misophonia, phonophobia and central auditory processing disorders).

In some embodiments the HCN2 inhibitor (preferably a compound of the invention) is for use in the treatment or prevention of tinnitus, Meniere's disease or hyperacusis. In some embodiments the HCN2 inhibitor is for use in the treatment or prevention of tinnitus or Meniere's disease. In a particular embodiment there is provided a compound of the invention, for use in the treatment or prevention of tinnitus.

### Migraine

The debilitating pain of migraine imposes a significant personal and economic burden. Actual or potential promise as therapeutics in migraine is shown by the triptan family, by the “gepant” family of antagonists to the CGRP receptor and by monoclonal antibodies against CGRP, amongst others. All have significant disadvantages, including the promotion of medication overuse headaches by triptans, liver toxicity in gepants and the need for regular injection of monoclonals. However, a significant fraction of migraine patients do not achieve relief with these treatments. There remains a need for new treatments for migraine.

Triptans are agonists at 5HT1B/D receptors, which couple to Gi/o and therefore inhibit production of cAMP5 (Alexander et al., Br. J. Pharmacol. 174 Suppl. 1, S17-S129, (2017)). The receptor for CGRP, which is emerging as a critical mediator of migraine, couples to Gs and therefore increases cAMP (Alexander et al. supra). These considerations suggest that cAMP in trigeminal nociceptive afferents innervating the meninges and dura may be a critical downstream mediator of migraine (Schytz et al., Curr. Opin. Neurol. 23, 259-265, (2010)).

As discussed herein, it has been shown that the HCN2 ion channel isoform, whose activation is potentiated by cAMP, promotes firing in nociceptive afferent neurons and, as a result, is a critical final effector of pain in animal models of nerve injury pain, of chemotherapy-induced pain and of painful diabetic neuropathy ((Tsantoulas, et al., Sci Transl Med 9, eaam6072, (2017); Tsantoulas et al., Biochem J 473, 2717-2736, 2016); Young et al., Pain 155, 1708-1719, (2014); and Emery et al., Science 333, 1462-1466, (2011)). Accordingly, HCN2 ion channels may be a critical downstream mediator of migraine pain. A HCN2 inhibitor may be useful in the treatment or prevention of migraine, particularly in the treatment or prevention of migraine pain.

In certain embodiments there is provided an HCN2 inhibitor for use in the prevention or treatment of migraine. In certain embodiments there is provided an HCN2 inhibitor for use in the treatment or prevention of migraine pain. In a preferred embodiment the HCN2 inhibitor is a compound of the invention. Accordingly there is provided a compound of the invention, for use in the prevention or treatment of migraine. Also provided is a compound of the invention, for use in the prevention or treatment of migraine pain.

### Treatment of Specific Pain Syndromes and Conditions

In certain embodiments a compound of the invention is for use in the treatment of a condition selected from: painful diabetic neuropathy; migraine rheumatoid arthritis (RA), osteoarthritis (OA), pain associated with long-term use of opioids (Opioid-induced hyperalgesia, OIH), cancer-associated bone pain and fibromyalgia (FMS, fibromyalgia syndrome).

### Subjects

A compound of the invention may be for use in the treatment of a human or animal subject affected by any of the medical conditions disclosed herein. The subject may be a warm-blooded mammal such as a farm animal (e.g. cow, sheep or pig) or a companion animal or pet (e.g. a dog, cat or horse). Preferably, the subject is a human.

### Combination Therapies

The methods of treatment according to the invention or the compound of the invention for use in the treatment of conditions mediated by HCN2 as defined herein may be applied as a sole therapy or be a combination therapy with an additional active agent.

For example, where the condition is pain (e.g. NP or IP) a compound of the invention maybe used in combination with another analgesic agent. Examples of analgesic agents include, but are not limited to an opioid (e.g. morphine and other opiate receptor agonists; nalbuphine or other mixed opioid agonist/antagonists; or tramadol); a non-steroidal anti-inflammatory agent (NSAIDs) (e.g. aspirin, ibuprofen, naproxen, or a selective COX2 inhibitor such as celecoxib); paracetamol; baclofen, pregabalin, gabapentin, a tricyclic antidepressant (e.g. clomipramine or amitriptyline), or a local anaesthetic (e.g. lidocaine), or a combination of two or more thereof.

The combination therapies defined herein may be achieved by way of the simultaneous, sequential or separate dosing of the individual components of the treatment. Such combination products employ the compounds of this invention within a therapeutically effective dosage range described herein and the other pharmaceutically-active agent within its approved dosage range.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

In some embodiments in which a combination treatment is used, the amount of the compound of the invention and the amount of the other pharmaceutically active agent(s) are, when combined, therapeutically effective to treat a targeted disorder in the patient. In this context, the combined amounts are “therapeutically effective amount” if they are, when combined, sufficient to reduce or completely alleviate symptoms or other detrimental effects of the disorder; cure the disorder; reverse, completely stop, or slow the progress of the disorder; or reduce the risk of the disorder getting worse. Typically, such amounts may be determined by one skilled in the art by, for example, starting with the dosage range described in this specification for the compound of the invention and an approved or otherwise published dosage range(s) of the other pharmaceutically active compound(s).

According to a further aspect of the invention there is provided a pharmaceutical product comprising a compound of the invention, or a pharmaceutically acceptable salt thereof as defined herein and an additional active agent for the treatment of pain (e.g. NP or IP). The additional active agent may be an analgesic agent as defined herein.

In an embodiment there is provided a pharmaceutical product comprising a compound of the invention, or a pharmaceutically acceptable salt thereof as defined herein and an additional active agent for the treatment of a condition which is modulated by HCN2. The additional active agent may be an analgesic agent as defined herein.

According to a further aspect of the invention there is provided a compound of the invention, or a pharmaceutically acceptable salt thereof for use simultaneously, sequentially or separately with an analgesic agent as defined herein, in the treatment of pain (e.g. NP or IP).

### Synthesis

In the description of the synthetic methods described below and in the referenced synthetic methods that are used to prepare the staring materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

Necessary starting materials may be obtained by standard procedures of organic chemistry. The preparation of such starting materials is described in conjunction with the following representative process variants and within the accompanying Examples. Alternatively necessary starting materials are obtainable by analogous procedures to those illustrated which are within the ordinary skill of an organic chemist.

It will be appreciated that during the synthesis of the compounds of the invention in the processes defined below, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl or trifluoroacetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example BF₃·OEt₂. A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

### General Synthetic Routes

Compounds of Formula (I) may be prepared according to Scheme 1.

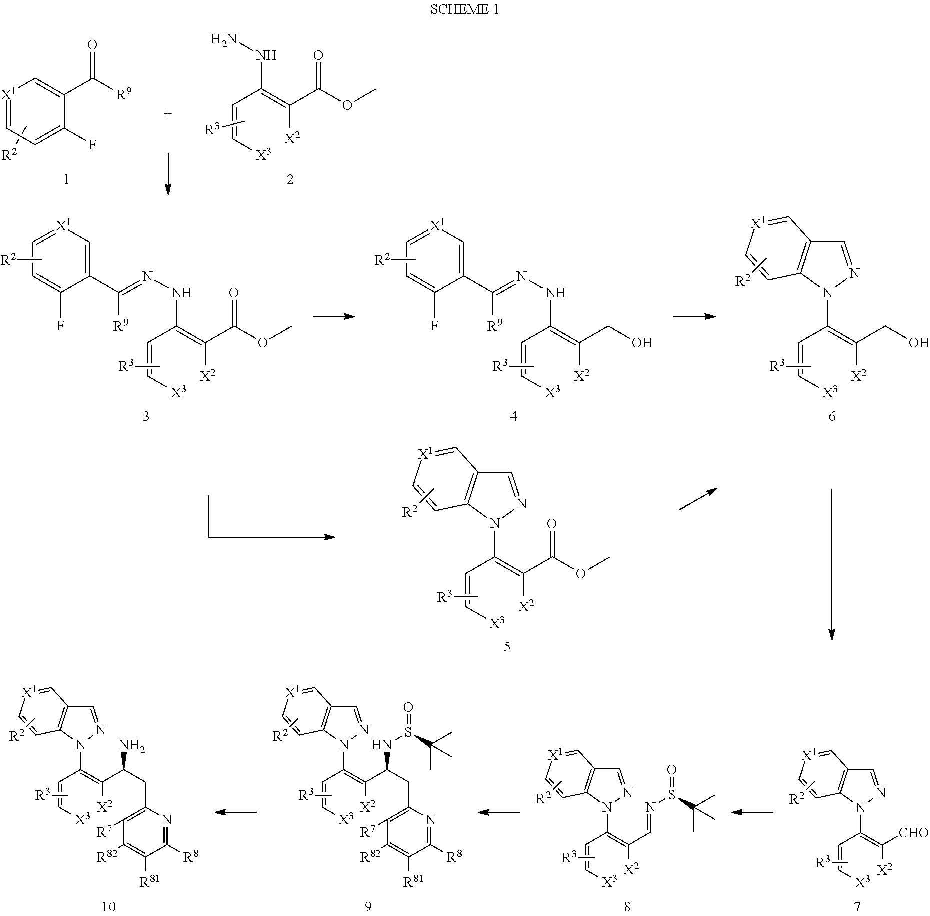

Intermediate hydrazones (3), may be prepared by the reaction of the appropriate aldehyde or ketone (1) with the appropriate hydrazine (2) optionally in the presence of a base such as cesium carbonate or potassium carbonate in a solvent such as an alcohol (methanol or ethanol) or DMF at a temperature between room temperature and the reflux temperature of the solvent. Conversion to the indazole alcohol analogue (6) could be achieved either by reduction to the alcohol followed by cyclisation or by performing the cyclisation first followed by the reduction. The reduction of the ester (3) to alcohol (4) could be carried out under a range of conditions known to one skilled in the art such as the use of DIBAL or lithium aluminium hydride in a solvent such as diethyl ether or THF at a temperature between −78° C. and room temperature. Cyclisation to the indazole can be achieved by treating the 2-fluorohydrazone (4) with a base such as potassium tert-butoxide in a polar solvent such as DMF or NMP at a temperature between room temperature and 100° C. Oxidation of the alcohol (4) to the aldehyde (5) may then be achieved using conditions known to one skilled in the art. For example, the oxidation may be carried out under Dess Martin periodinane conditions in DCM at room temperature or under Swern conditions using oxalyl chloride and DMSO in a solvent such as DCM in the presence of a base such as a trialkylamine base, for example triethylamine.

Conversion of the aldehyde (7) to the imine (8) may be achieved by treatment with the appropriate chiral single enantiomer of (S)-2-methylpropane-2-sulfinamide in the presence of a base such as cesium carbonate in a chlorinated solvent such as DCM, at the reflux temperature of the solvent. Alternatively, reaction with (S)-2-methylpropane-2-sulfinamide may be carried out in the presence of for example titanium ethoxide in an appropriate solvent such as ethanol or THF at a temperature between room temperature and the reflux temperature of the solvent. Reaction of the generated single enantiomer of the sulfinamide (8) with the anion generated from the appropriately substituted 2-alkyl pyridine and an organolithium reagent such as n-butyl lithium, lithium di-isopropylamide or lithium hexamethyl disilazide in a solvent such as THF at a temperature between −78° C. and 0° C. gives preferentially the desired diastereomeric isomer of the intermediate (9) which can be purified by chromatography to remove the undesired minor diastereomer. Deprotection under acidic conditions using for example HCl or trifluoroacetic acid in a solvent such as DCM, ethyl acetate methanol or dioxane then provides the target compounds of Formula (I) as single enantiomers (10).

One skilled in the art will recognise that interconversion of various groups such as R, R² or R³ may be carried out at different stages of the synthesis and that protection of various functionalities may be required in order to complete the required syntheses.

An alternative synthetic approach to compounds of Formula (I) via the same intermediate aldehyde (7) is described in Scheme 2.

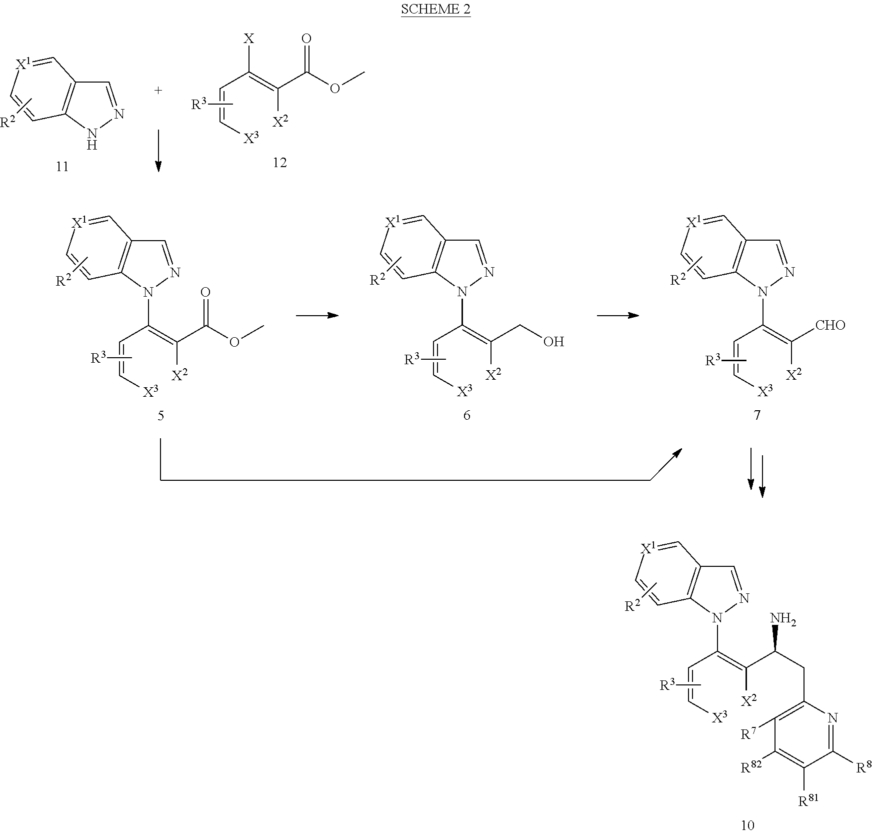

Intermediate indazoles (5) wherein X² or X³ is N may be prepared by the reaction of the N-unsubstituted indazoles (11) with a 2-halo substituted carboxylic ester (12), for example where X is F or C in the presence of a strong base, such as sodium hydride in a solvent such as THF or DMF at a temperature between 0° C. and room temperature. Removal of the undesired regioisomer may be achieved by chromatography. Alternatively, where X² and X³ are carbon, then X is bromine or iodine and the reaction may be carried out under copper catalysed conditions using for example copper (I) iodide or copper (I) oxide, in the presence of a base such as potassium phosphate, potassium hydroxide or cesium carbonate and an amine such as dimethylethylenediamine or 1,2-cyclohexanediamine in a solvent such as DMF or dioxane at a temperature between room temperature and the reflux temperature of the solvent. One skilled in the art will recognise that there are a number of other conditions that may be used for the coupling of the halo ester with the indazole to generate intermediates of the type (5)

Conversion of the ester (5) to the aldehyde (7) may be achieved either directly or in two steps. Direct reduction of the ester to the aldehyde may be achieved using, for example, a mild reducing agent such as DIBAL in a solvent such as DCM at a temperature between −78° C. and room temperature. Alternatively, the ester (5) may be reduced to the alcohol (6) using stronger conditions such as, for example, lithium aluminium hydride in an ether solvent such as diethyl ether or THF at a temperature between −78° C. and room temperature. Oxidation of the alcohol (6) to the aldehyde (7) may then be achieved using conditions known to one skilled in the art. For example the oxidation may be achieved using Dess Martin periodinane conditions in DCM at room temperature or under Swern conditions using oxalyl chloride and DMSO in a solvent such as DCM in the presence of a base such as a trialkylamine base, for example triethylamine.

With the aldehyde (7) in hand, conversion to the compounds of Formula (I) may be achieved using the same methods as described above in Scheme 1.

### EXAMPLES

### Abbreviations:

The following abbreviations are used:
DCM: Dichloromethane
DIBAL: Di-isobutylaluminium hydride
DMF: N,N-Dimethyl formamide
DMP: Dess Martin periodinane
DMSO: Dimethyl sulfoxide
EGTA: Ethylene glycol-bis(2-aminoethylether)-N,N,N′,N′-tetraacetic acid
FCC: Flash column chromatography
HATU: O-(7-Azabenzotriazol-1-yl)-N,N,N,N-tetramethyl uronium hexafluorophosphate
HEPES: 4-(2-hydroxyethyl)-1-piperazineethanesulfonic acid
HPLC: High performance liquid chromatography
IPA: 2-Propanol
LDA: Lithium di-isopropylamide
MDAP: Mass-directed autopurification
Min: Minutes
MP-carbonate: Tetraalkylammoniumj carbonate polymer bound (macro-porous)
NMP: N-methylpyrrolidinone
RT: Retention time
TBME: tert-butyl methyl ether
TFA: Trifluoroacetic acid
THF: Tetrahydrofuran

In the procedures that follow, after each starting material, reference to an Intermediate/Example number is usually provided. This is provided merely for assistance to the skilled chemist. When reference is made to the use of a “similar” or “analogous” procedure, as will be appreciated by those skilled in the art, such a procedure may involve minor variations, for example reaction temperature, reagent/solvent amount, reaction time, work-up conditions or chromatographic purification conditions.

NMR spectra were obtained on a Varian Unity Inova 400 spectrometer with a 5 mm inverse detection triple resonance probe operating at 400 MHz or on a Bruker Avance DRX 400 spectrometer with a 5 mm inverse detection triple resonance TXI probe operating at 400 MHz or on a Bruker Avance DPX 300 spectrometer with a standard 5 mm dual frequency probe operating at 300 MHz or on a Bruker Fourier 300 spectrometer with a 5 mm dual frequency probe operating at 300 MHz. Shifts are given in ppm relative to tetramethylsilane (5=0 ppm). J values are given in Hz through-out. NMR spectra were assigned using CMC-Assist Version 2.3 or SpinWorks version 3.

Liquid chromatography mass spectroscopy (LCMS) methods used are as follows.

### Method 1:

| Instrumentation | HQC - Acquity UPLC (binary pump/PDA detector) + | | |
|---|---|---|---|
| | ZQ Mass Spectrometer | | |
| Column | ACQUITY UPLC BEH C₁₈ 1.7 μm, 100 × 2.1 mm, | | |
| | maintained at 40° C. | | |
| Mobile Phase A | 0.1% Aqueous formic acid (v/v) | | |
| Mobile Phase B | 0.1% Formic acid in acetonitrile (v/v) | | |
| Flow | 0.4 ml/min | | |
| Gradient Program | Time (mins) | % A | % B |
| | 0.0 | 95 | 05 |
| | 0.4 | 95 | 05 |
| | 6.0 | 05 | 95 |
| | 6.8 | 05 | 95 |
| | 7.0 | 95 | 05 |
| | 8.0 | 95 | 05 |

| | |
|---|---|
| Sample | 1 μl injection of a 0.2-0.5mg/ml solution in an |
| | appropriate solvent at 20° C. |
| Detectors | UV, diode array 200-500 nm |
| | MS, mass 100-800 (or −1500 for HM method) in |
| | ES+ & ES− (no split to MS) |
| Data Analysis | Peak area percentage (APCT) with an integration |
| | threshold of 0.2% (relative) |

| Instrumentation | HPS - Acquity i-Class (quarternary pump/PDA | | |
|---|---|---|---|
| | detector) + Quattro Micro Mass Spectrometer | | |
| Column | ACQUITY UPLC BEH C₁₈ 1.7 μm, 100 × 2.1 mm, | | |
| | maintained at 40° C. | | |
| Mobile Phase A | 0.1% Aqueous formic acid (v/v) | | |
| Mobile Phase B | 0.1% Formic acid in acetonitrile (v/v) | | |
| Flow | 0.4 ml/min | | |
| Gradient Program | Time (mins) | % A | % B |
| | 0.0 | 95 | 05 |
| | 0.4 | 95 | 05 |
| | 6.0 | 05 | 95 |
| | 6.8 | 05 | 95 |
| | 7.0 | 95 | 05 |
| | 8.0 | 95 | 05 |

| | |
|---|---|
| Sample | 1 μl injection of a 0.5 mg/ml solution in an |
| | appropriate solvent at 20° C. |
| Detectors | UV, diode array 200-500 nm |
| | MS, mass 100-800 (or −1500 for HM method) in |
| | ES+ & ES− (no split to MS) |

### Method 2:

| Instrumentation | HP1100 (quaternary pump/PDA detector) + ZQ | | |
|---|---|---|---|
| | Mass Spectrometer | | |
| Column | Phenomenex Luna C18(2) 3μ, 30 × 4.6 mm | | |
| Mobile Phase A | 0.1% Aqueous formic acid (v/v) | | |
| Mobile Phase B | 0.1% Formic acid in acetonitrile (v/v) | | |
| Flow | 2.0 ml/min | | |
| Gradient Program | Time (mins) | % A | % B |
| | 0.0 | 95 | 05 |
| | 0.3 | 95 | 05 |
| | 4.3 | 05 | 95 |
| | 5.3 | 05 | 95 |
| | 5.8 | 95 | 05 |
| | 6.0 | 95 | 05 |

| | |
|---|---|
| Detectors | UV, diode array 190-450 nm |
| | MS, mass 160-1000 (or 100-800 or 160-1300) in |
| | ES+ & ES− (200 μl/min split to MS) |

### Method 3:

MDAP methods used were as follows.

MDAP Method (standard—acidic):
Agilent Technologies 1260 Infinity purification system with an XSELECT CSH Prep C18 column (19×250 mm, 5 μm OBD) maintained at RT
Mobile Phase A: 0.1% aqueous formic acid
Mobile Phase B: 0.1% formic acid in acetonitrile
Flow Rate: 20 ml/min
Gradient Program: 10%-95%, 22 min, centered around a specific focused gradient
Sample: Injection of a 20-60 mg/mL solution in DMSO (+optional formic acid and water)

### MDAP Method (Basic):

Agilent Technologies 1260 Infinity purification system with an XBridge Prep C18 OBD column (19×250 mm, 5 μm OBD) maintained at RT
Mobile Phase A: 0.1% aqueous ammonia
Mobile Phase B: 0.1% ammonia in acetonitrile
Flow Rate: 20 ml/min
Gradient Program: 10%-95%, 22 min, centered around a specific focused gradient
Sample: Injection of a 20-60 mg/ml solution in DMSO+optional formic acid and water)

### Example 1: (S)-1-[2-(5-Bromo-1H-indazol-1-yl)phenyl]-2-(pyridine-2-yl)ethane-1-amine hydrochloride

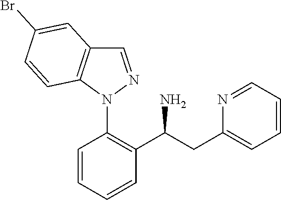 [SMILES: N[C@@H](Cc1ccccn1)c1ccccc1-n1ncc2cc(Br)ccc21]

### Intermediate 1A: Methyl 2-[2-(5-bromo-2-fluorobenzylidene)hydrazineyl]benzoate

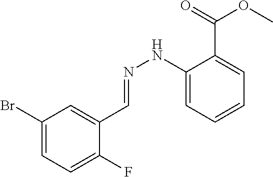 [SMILES: COC(=O)c1ccccc1N/N=C/c1cc(Br)ccc1F]

A mixture of 5-bromo-2-fluorobenzaldehyde (4.32 g), methyl 2-hydrazineylbenzoate (prepared according to Australian Journal of Chemistry vol. 69(11) 1268, 4.32 g) and cesium carbonate (6.95 g) in DMF (80 mL) was stirred at room temperature overnight. The mixture was poured in rapidly stirring water and the resultant solids were collected by filtration and dried in vacuo to give the title compound as a yellow solid (7.0 g).

¹H NMR (400 MHz, DMSO-ds) 11.20 (1H, s), 8.32 (1H, s), 8.07 (1H, dd, J=2.5, 6.6 Hz), 7.88-7.78 (2H, m), 7.59-7.53 (2H, m), 7.27 (1H, dd, J=8.8, 10.4 Hz), 6.92-6.87 (1H, m), 3.88 (3H, s);

### Intermediate 1B: {2-[2-(5-Bromo-2-fluorobenzylidene)hydrazineyl]phenyl}methanol

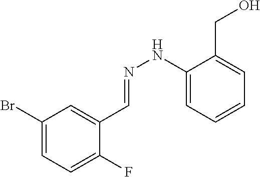 [SMILES: OCc1ccccc1N/N=C/c1cc(Br)ccc1F]

Lithium aluminium hydride (0.397 g) was added in portions to a stirred, cooled solution of methyl 2-[2-(5-bromo-2-fluorobenzylidene)hydrazineyl]benzoate (Intermediate 1A, 3.5 g) in dry THF (40 mL) while maintaining the temperature below 5° C. On completion of the addition, the temperature was allowed to rise to room temperature and the mixture was stirred for 4.5 hours then re-cooled to 0° C. The reaction mixture was quenched by addition of water (0.4 mL) followed by 15% aqueous sodium hydroxide (0.4 mL) and finally water (1.2 mL) then filtered through Celite™. The filtrate was concentrated in vacuo and the residue was purified by FCC eluting with 0-30% ethyl acetate in petroleum ether to give the title compound as an orange solid (2.0 g).

LCMS (Method 3) RT 3.82 m/z 305/307 [MH⁺−18]

### Intermediate 1C: [2-(5-Bromo-1H-indazol-1-yl)phenyl]methanol

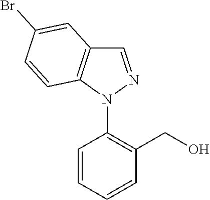 [SMILES: OCc1ccccc1-n1ncc2cc(Br)ccc21]

Potassium tert-butoxide (0.47 g) was added to a solution of {2-[2-(5-bromo-2-fluorobenzylidene)hydrazineyl]phenyl}methanol (Intermediate 1B, 0.542 g) in NMP (7 mL) and the resultant mixture was stirred and heated at 100° C. overnight. After cooling, the mixture was added to water and the pH was adjusted to −5 by addition of potassium bisulfate then extracted with ethyl acetate. The organic layer was washed with water dried (MgSO₄) and filtered and the filtrate was concentrated in vacuo. The crude product was combined with a second experiment using {2-[2-(5-bromo-2-fluorobenzylidene)hydrazineyl]phenyl}methanol (Intermediate 1B, 0.581 g) and purified by FCC eluting with 0-50% TBME in petroleum ether to give the title compound (0.198 g).

LCMS (Method 3) RT 3.32 m/z 285/287 [MH*-18]

### Intermediate 1D: 2-(5-Bromo-1H-indazol-1-yl)benzaldehyde

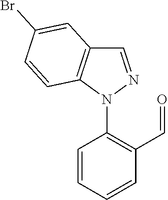 [SMILES: O=Cc1ccccc1-n1ncc2cc(Br)ccc21]

DMP (0.257 g) was added to a solution of [2-(5-bromo-1H-indazol-1-yl)phenyl]methanol (Intermediate 1C, 0.153 g) in DCM (4 mL) and the mixture was stirred at room temperature under argon overnight. Aqueous sodium thiosulfate solution was added and the mixture was extracted with DCM, washed with saturated sodium bicarbonate solution and brine then dried (MgSO₄) and filtered. The filtrate was concentrated in vacuo to give the title compound as a brown solid (0.198 g).

LCMS (Method 3) RT 3.67 m/z 301/303 [MH⁺]

### Intermediate 1E: (S,E)-N-[2-(5-Bromo-1H-indazol-1-yl)benzylidene]-2-methylpropane-2-sulfinamide

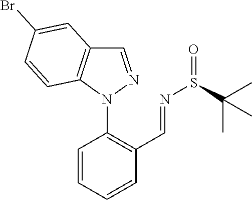 [SMILES: CC(C)(C)[S@](=O)/N=C/c1ccccc1-n1ncc2cc(Br)ccc21]

A mixture of 2-(5-bromo-1H-indazol-1-yl)benzaldehyde (Intermediate 1D, 0.198 g), (S)-2-methylpropane-2-sulfinamide (0.095 g) and cesium carbonate (0.257 g) in DCM (5 mL) was stirred and heated at gentle reflux overnight. The resultant mixture was concentrated in vacuo and the residue was partitioned between water and ethyl acetate. The layers were separated and the aqueous layer was further extracted with ethyl acetate. The combined organic layers were dried (MgSO₄) and filtered. The filtrate was concentrated in vacuo and the residue was purified by FCC eluting with 0-30% ethyl acetate in petroleum ether to give the title compound (0.124 g).

¹H NMR (400 MHz, CDCl3) 8.40 (1H, s), 8.25 (1H, dd, J=1.3, 8.0 Hz), 8.19 (1H, d, J=0.9 Hz), 7.96-7.95 (1H, m), 7.67 (1H, ddd, J=7.6, 7.6, 1.5 Hz), 7.60-7.57 (1H, m), 7.57-7.53 (1H, m), 7.47 (1H, dd, J=1.7, 9.0 Hz), 7.25-7.23 (1H, m), 1.21 (9H, s);

### Intermediate 1F: (S)-N-{(S)-1-[2-(5-Bromo-1H-indazol-1-yl)phenyl]-2-[pyridine-2-yl]ethyl}-2-methylpropane-2-sulfinamide

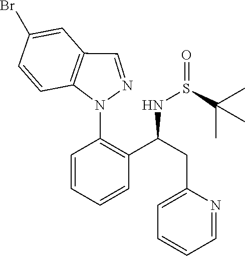 [SMILES: CC(C)(C)[S@](=O)N[C@@H](Cc1ccccn1)c1ccccc1-n1ncc2cc(Br)ccc21]

n-Butyllithium (2.5M in hexanes, 0.1 mL) was added to a stirred, cooled solution of 2-methylpyrindine (0.024 mL) in THF (1 mL) while maintaining the temperature below −70° C. On completion of the addition, the mixture was stirred at −78° C. for 15 minutes then a solution of (S,E)-N-[2-(5-bromo-1H-indazol-1-yl)benzylidene]-2-methylpropane-2-sulfinamide (Intermediate 1E, 0.045 g) in THF (1 mL) was added. The mixture was stirred at −78° C. for 30 minutes then allowed to warm to −30° C. Saturated ammonium chloride was added and the mixture was allowed to warm to room temperature and partitioned between water and ethyl acetate. The layers were separated and the aqueous layer was further extracted with ethyl acetate and the combined organic layers were dried (MgSO₄) and filtered. The filtrate was concentrated in vacuo and the residue was purified by FCC eluting with 0-100% ethyl acetate in petroleum ether followed by 0-2% methanol in ethyl acetate to give the title compound (0.041 g).

¹H NMR (400 MHz, CDCl3) 8.42 (1H, dd, J=0.8, 4.8 Hz), 8.20 (1H, d, J=1.0 Hz), 7.97 (1H, d, J=1.3 Hz), 7.68 (1H, dd, J=1.5, 7.8 Hz), 7.48-7.42 (3H, m), 7.40 (1H, dt, J=1.2, 7.5 Hz), 7.31 (1H, dd, J=1.6, 8.1 Hz), 7.22 (1H, d, J=8.9 Hz), 7.06 (1H, ddd, J=1.1, 4.9, 7.5 Hz), 6.78 (1H, d, J=7.6 Hz), 5.56 (1H, d, J=6.8 Hz), 4.67-4.60 (1H, m), 3.11 (1H, m), 2.94-2.87 (1H, m), 0.97 (9H, s);

### Example 1: (S)-1-[2-(5-Bromo-1H-indazol-1-yl)phenyl]-2-(pyridine-2-yl)ethane-1-amine hydrochloride

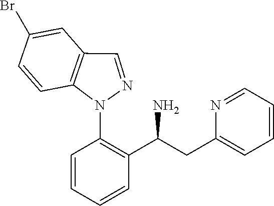 [SMILES: N[C@@H](Cc1ccccn1)c1ccccc1-n1ncc2cc(Br)ccc21]

Hydrogen chloride in dioxane (4M, 0.18 mL) was added to a solution of (S)-N-{(S)-1-[2-(5-bromo-1H-indazol-1-yl)phenyl]-2-[pyridine-2-yl]ethyl}-2-methylpropane-2-sulfinamide (Intermediate 1F, 0.04 g) in methanol (0.5 mL) and the mixture was stirred for 1 hour then concentrated in vacuo. The residue was purified by MDAP under acidic conditions and the purified material was dissolved in methanol and MP-carbonate was added. The mixture was allowed to stand for 1 hour then filtered and the solid was washed with methanol. The filtrate was concentrated in vacuo and the residue was dissolved in dioxane and treated with hydrogen chloride in dioxane (4M) and concentrated in vacuo. The residue was redissolved in water and freeze dried to give the title compound as a white solid (0.02 g).

¹H NMR (400 MHz, DMSO-ds) 8.79 (3H, br s), 8.32 (1H, d, J=0.9 Hz), 8.17-8.13 (1H, m), 8.12-8.06 (2H, m), 7.72-7.62 (2H, m), 7.55 (1H, dt, J=1.2, 7.8 Hz), 7.48 (1H, dd, J=1.7, 8.8 Hz), 7.43 (1H, dd, J=1.2, 7.9 Hz), 7.23-7.16 (1H, m), 7.08 (2H, d, J=8.8 Hz), 4.77-4.66 (1H, m), 3.46-3.40 (1H, m), 3.39-3.30 (1H, m).

LCMS (Method 1) RT 3.22 m/z 393/395 [MH⁺]

### Example 2: (S)-6-{2-[3-(1H-indazol-1-yl)pyridine-2-yl]-2-aminoethyl}-5-methylpyridin-2-carbonitrile hydrochloride

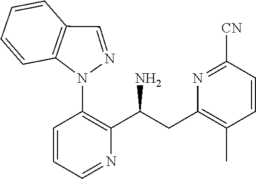 [SMILES: Cc1ccc(C#N)nc1C[C@H](N)c1ncccc1-n1ncc2ccccc21]

### Intermediate 2A: Ethyl 3-(1H-indazol-1-yl)pyridine-2-carboxylate

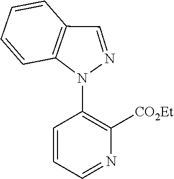 [SMILES: CCOC(=O)c1ncccc1-n1ncc2ccccc21]

Sodium hydride (60% oil dispersion, 0.4 g) was added in portions to a cooled solution of 1H-indazole (1.18 g) in DMF (20 mL) at 0° C. The resultant mixture was stirred in the ice bath until effervescence ceased. The cooling bath was removed and ethyl 3-fluoropyridine-2-carboxylate (1.52 g) was added. The resultant mixture was stirred for 6 hours at room temperature then heated at 70° C. for 2.5 hours. After cooling, the mixture was diluted with water and brine and extracted with ethyl acetate, washed with brine, dried (Na₂SO₄) and filtered. The filtrate was concentrated in vacuo and the residue was purified by FCC eluting with 0-50% ethyl acetate in pentane to give the title compound (0.81 g).

¹H NMR (400 MHz, CDCl₃) 8.77 (1H, dd, J=1.5, 4.7 Hz), 8.23-8.23 (1H, s), 8.05 (1H, dd, J=1.5, 8.1 Hz), 7.83-7.81 (1H, m), 7.63 (1H, dd, J=4.7, 8.1 Hz), 7.44-7.42 (2H, m), 7.28-7.23 (1H, m), 4.15-4.09 (2H, m), 0.99-0.95 (3H, m);

### Intermediate 2B: 3-(1H-Indazole-1-yl)pyridine-2-carboxaldehyde

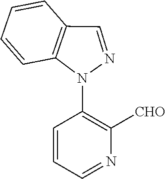 [SMILES: O=Cc1ncccc1-n1ncc2ccccc21]

DIBAL (1M solution in DCM, 18 mL) was added dropwise to a stirred, cooled solution of ethyl 3-(1H-indazol-1-yl)pyridine-2-carboxylate (Intermediate 2A, 3.45 g) in DCM (77 mL) while maintaining the temperature below −60° C. The mixture was stirred at −78° C. for a further 1.5 hours then methanol (5 mL) was added. The resultant mixture was again stirred at −78° C. for 2.5 hours then allowed to warm to room temperature. It was filtered through Celite™ and the filtrate was concentrated in vacuo. The residue was purified by FCC eluting with 0-100% ethyl acetate in DCM to give the title compound as a yellow oil which solidified on standing (2.3 g).

¹H NMR (400 MHz, CDCl₃) 10.07 (1H, s), 8.91 (1H, dd, J=1.4, 4.5 Hz), 8.30 (1H, s), 8.07 (1H, dd, J=1.5, 8.1 Hz), 7.88-7.85 (1H, m), 7.70 (1H, dd, J=4.6, 8.1 Hz), 7.50-7.45 (1H, m), 7.38 (1H, dd, J=0.6, 8.5 Hz), 7.33-7.28 (1H, m);

### Intermediate 2C: (S,E)-N-{[3-(1H-indazol-1-yl)pyridine-2-yl]methylene}-2-methylpropane-2-sulfinamide

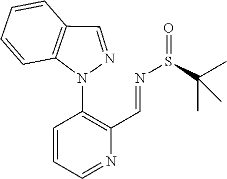 [SMILES: CC(C)(C)[S@](=O)/N=C/c1ncccc1-n1ncc2ccccc21]

Prepared by proceeding in a similar manner to Intermediate 1E, starting from 3-(1H-indazole-1-yl)pyridine-2-carboxaldehyde (Intermediate 2B) and (S)-2-methylpropane-2-sulfinamide.

¹H NMR (400 MHz, CDCl₃) 8.91 (1H, dd, J=1.5, 4.6 Hz), 8.58 (1H, s), 8.27-8.27 (1H, m), 7.98 (1H, dd, J=1.6, 8.1 Hz), 7.84-7.81 (1H, m), 7.61 (1H, dd, J=4.6, 8.1 Hz), 7.45-7.40 (1H, m), 7.33-7.28 (1H, m), 7.27-7.25 (1H, m), 1.16 (9H, s);

### Intermediate 2D: (S)-N-{(S)-1-[3-(1H-indazol-1-yl)pyridine-2-yl]-2-[6-bromo-2-methylpyridine-2-yl]ethyl}-2-methylpropane-2-sulfinamide

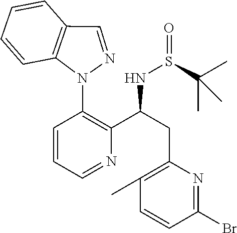 [SMILES: Cc1ccc(Br)nc1C[C@H](N[S@@](=O)C(C)(C)C)c1ncccc1-n1ncc2ccccc21]

LDA (2M in THF, heptane, ethylbenzene, 1 mL) was added dropwise to a stirred, cooled solution of 6-bromo-2,3-dimethylpyridine (0.4 g) in THF (8 mL) while maintaining the temperature below −60° C. On completion of the addition, the mixture was stirred at −78° C. for 1 hour then added by cannula to a precooled (−78° C.) solution of (S,E)-N-{[3-(1H-indazol-1-yl)pyridine-2-yl]methylene}-2-methylpropane-2-sulfinamide (Intermediate 2C, 0.25 g) in THF (4 mL). The resultant mixture was stirred at −78° C. for 1 hour then allowed to warm to room temperature. It was partitioned between ethyl acetate and water and the organic layer was dried (Na₂SO₄) and filtered. The filtrate was concentrated in vacuo and the residue was purified by FCC eluting with 0-5% methanol in DCM to give the title compound (0.305 g).

¹H NMR (400 MHz, CDCl₃) 8.75-8.72 (1H, m), 8.23 (1H, s), 7.84-7.81 (1H, m), 7.71-7.68 (1H, m), 7.52-7.43 (2H, m), 7.41-7.37 (1H, m), 7.29-7.23 (1H, m), 7.06-7.05 (2H, m), 5.28-5.24 (1H, m), 5.02-4.95 (1H, m), 3.18 (1H, dd, J=5.9, 13.4 Hz), 3.09 (1H, dd, J=9.1, 13.8 Hz), 1.77 (3H, s), 1.06-1.05 (9H, s);

### Intermediate 2E: (S)-N-{(S)-1-[3-(1H-indazol-1-yl)pyridine-2-yl]-2-[6-cyano-2-methylpyridine-2-yl]ethyl}-2-methylpropane-2-sulfinamide

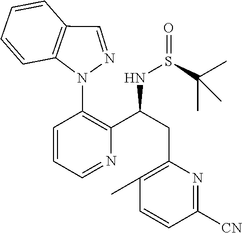 [SMILES: Cc1ccc(C#N)nc1C[C@H](N[S@@](=O)C(C)(C)C)c1ncccc1-n1ncc2ccccc21]

A mixture of (S)-N-{(S)-1-[3-(1H-indazol-1-yl)pyridine-2-yl]-2-[6-bromo-2-methylpyridine-2-yl]ethyl}-2-methylpropane-2-sulfinamide (Intermediate 2D, 0.305 g) zinc cyanide (0.21 g) and tetrakis(triphenylphosphine) palladium (0.139 g) in DMF (5 mL) was degassed and heated in a sealed vial at 90° C. for 2 hours. After cooling, the mixture was filtered through Celite™ and washed with ethyl acetate. The filtrate was concentrated in vacuo to give the title compound (0.27 g).

¹H NMR (400 MHz, CDCl₃) 8.75-8.72 (1H, m), 8.24 (1H, s), 7.86-7.83 (1H, m), 7.74-7.70 (1H, m), 7.50-7.47 (2H, m), 7.41 (1H, dd, J=4.4, 8.0 Hz), 7.37-7.34 (1H, m), 7.30-7.27 (2H, m), 5.24-5.19 (1H, m), 5.09-4.98 (1H, m), 3.28 (1H, dd, J=5.9, 14.0 Hz), 3.19 (1H, dd, J=8.7, 14.0 Hz), 2.03 (3H, s), 1.05 (9H, s);

### Example 2: (S)-6-{2-[3-(1H-indazol-1-yl)pyridine-2-yl]-2-aminoethyl}-5-methylpyridin-2-carbonitrile hydrochloride

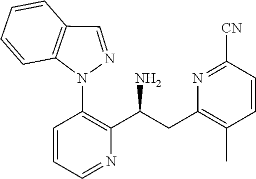 [SMILES: Cc1ccc(C#N)nc1C[C@H](N)c1ncccc1-n1ncc2ccccc21]

Prepared by proceeding in a similar manner to Example 1, starting from (S)-N-{(S)-1-[3-(1H-indazol-1-yl)pyridine-2-yl]-2-[6-cyano-2-methylpyridine-2-yl]ethyl}-2-methylpropane-2-sulfinamide (Intermediate 2E) and converting to the HCl salt by dissolving in acetonitrile, treating with 0.1M aqueous HCl and freeze drying.

¹H NMR (400 MHz, DMSO-ds) 8.87 (1H, dd, J=1.4, 4.7 Hz), 8.61 (3H, s), 8.38-8.37 (1H, m), 8.07 (1H, dd, J=1.4, 8.1 Hz), 7.89-7.86 (1H, m), 7.72 (1H, dd, J=4.7, 8.1 Hz), 7.50-7.46 (1H, m), 7.43-7.39 (1H, m), 7.36-7.34 (1H, m), 7.31-7.26 (1H, m), 7.23-7.20 (1H, m), 5.30-5.24 (1H, m), 3.27-3.23 (2H, m), 1.94-1.93 (3H, m);

LCMS (Method 1) RT 2.84 m/z 355 [MH⁺]

The compounds in Table 3 were prepared using similar methods to those described for Examples 1 and 2.

TABLE 3

| No | Structure | Name | NMR | LCMS |
|---|---|---|---|---|
| 3 | 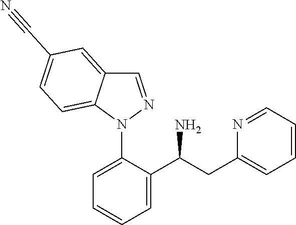 [SMILES: N#Cc1ccc2c(cnn2-c2ccccc2[C@@H](N)Cc2ccccn2)c1] | (S)-1-{2-[1-Amino-2- (pyridine-2-yl)ethyl]- phenyl}-1H-indazole- 5-carbonitrile hydro- chloride | ¹H NMR (400 MHz, DMSO- d₆) 8.76 (3H, br s), 8.56-8.49 (2H, m), 8.11-8.03 (2H, m), 7.73-7.61 (3H, m), 7.58 (1H, dt, J = 1.4, 7.9 Hz), 7.46 (1H, dd, J = 1.3, 7.9 Hz), 7.22-7.13 (2H, m), 7.07-7.02 (1H, m), 4.73-7.65 (1H, m), plus 2 protons under the water peak | (Method 1) RT 2.58 min m/z 340 [MH⁺] |
| 4 | 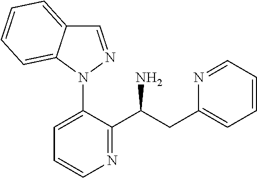 [SMILES: N[C@@H](Cc1ccccn1)c1ncccc1-n1ncc2ccccc21] | (S)-1-[3-(1H-indazole- 1-yl)pyridine-2-yl]-2- (pyridine-2-yl)ethan-1- amine hydrochloride | ¹H NMR (400 MHz, DMSO- d₆) 8.84 (1H, dd, J = 1.4, 4.8 Hz), 8.80 (3H, s), 8.39 (1H, s), 8.26-8.21 (1H, m), 8.06 (1H, dd, J = 1.5, 8.1 Hz), 7.91-7.88 (1H, m), 7.82-7.74 (1H, m), 7.70 (1H, dd, J = 4.7, 8.1 Hz), 7.48-7.42 (1H, m), 7.32-7.28 (3H, m), 7.19- 7.13 (1H, m), 5.14-5.14 (1H, m), 3.49-3.35 (2H, m); | (Method 1) RT 2.49 min m/z 316 [MH⁺] |
| 5 | 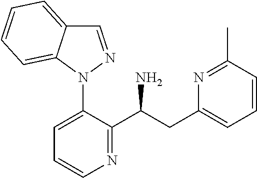 [SMILES: Cc1cccc(C[C@H](N)c2ncccc2-n2ncc3ccccc32)n1] | (S)-1-[3-(1H-indazole- 1-yl)pyridine-2-yl]-2- (6-methylpyridine-2- yl)ethan-1-amine hydrochloride | ¹H NMR (400 MHz, DMSO- d₆) 8.77-8.73 (1H, m), 8.36 (1H, s), 7.88-7.82 (2H, m), 7.51 (1H, dd, J = 4.7, 7.9), 7.38-7.32 (1H, m), 7.31-7.20 (2H, m), 7.09-7.05 (1H, m), 6.77 (1H, d, J = 7.5 Hz), 6.53 (1H, d, J = 7.8 Hz), 4.51-4.44 (1H, m), 3.07-3.00 (1H, m), 2.92-2.84 (1H, m), 2.01 (3H, s); | (Method 1) RT 2.17 min m/z 330 [MH⁺] |
| 6 | 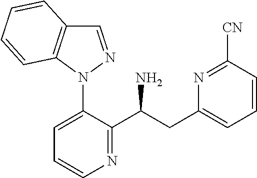 [SMILES: N#Cc1cccc(C[C@H](N)c2ncccc2-n2ncc3ccccc32)n1] | (S)-6-{2-[3-(1H-ind- azol-1-yl)pyridine-2-yl]- 2-aminoethyl}pyridine- 2-carbonitrile hydro- chloride | ¹H NMR (400 MHz, DMSO- d₆) 8.82 (1H, dd, J = 1.6, 4.8 Hz), 8.54 (3H, br s), 8.38 (1H, d, J = 0.8 Hz), 8.02 (1H, dd, J = 1.6, 8.2 Hz), 7.88- 7.84 (1H, m), 7.69-7.61 (2H, m), 7.48-7.44 (1H, m), 7.41- 7.36 (1H, m), 7.28-7.23 (1H, m), 7.18-7.12 (2H, m), 5.16- 5.09 (1H, m), 3.24-3.19 (2H, m); | (Method 2) RT 2.63 min m/z 341 [MH⁺] |
| 7 | 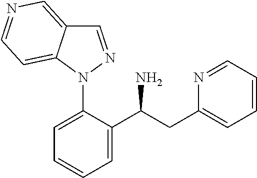 [SMILES: N[C@@H](Cc1ccccn1)c1ccccc1-n1ncc2cnccc21] | (S)-1-[2-(1H-pyrazolo- [4,3-c]pyridine-1-yl)- phenyl]-2-(pyridine-2- yl)ethan-1-amine hydrochloride | ¹H NMR (400 MHz, DMSO- d₆) 8.62 (1H, s), 8.37 (1H, d, J = 6.1 Hz), 8.06-7.99 (2H, m), 7.69-73.64 (1H, m), 7.57- 7.47 (2H, m), 7.43 (1H, d, J = 7.9 Hz), 7.11 (1H, d, J = 5.8 Hz), 7.05-7.00 (1H, m), 6.89 (1H, d, J = 8.1 Hz), 4.52-4.46 (1H, m), 3.14-3.09 (2H, m); | (Method 2) RT 3.16 min m/z 316 [MH⁺] |
| 8 | 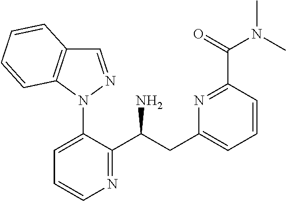 [SMILES: CN(C)C(=O)c1cccc(C[C@H](N)c2ncccc2-n2ncc3ccccc32)n1] | (S)-6-{2-[3-(1H-ind- azol-1-yl)pyridine-2-yl]- 2-aminoethyl}-N,N- dimethylpyridine-2- carboxamide hydro- chloride | ¹H NMR (400 MHz, DMSO- d₆) 8.80 (1H, dd, J = 1.5, 4.7 Hz), 8.40 (1H, d, J = 0.9 Hz), 7.98 (1H, dd, J = 1.5, 7.9 Hz), 7.93-7.88 (1H, m), 7.64-7.58 (2H, m), 7.46 7.41 (1H, m), 7.32-7.25 (3H, m), 6.99-6.97 (1H, m), 5.70 (2H, s), 4.57- 4.51 (1H, m), 3.22 (1H, dd, J = 6.1, 13.8 Hz), 3.07 (1H, dd, J = 7.7, 13.8 Hz), 2.90 (3H, s), 2.55 (3H, s); | (Method 2) RT 2.56 min m/z 387 [MH⁺] |
| 9 | 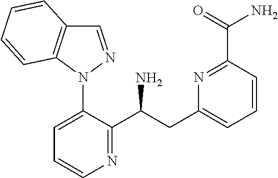 [SMILES: NC(=O)c1cccc(C[C@H](N)c2ncccc2-n2ncc3ccccc32)n1] | (S)-6-{2-[3-(1H-ind- azol-1-yl)pyridine-2-yl]- 2-aminoethyl}pyridine- 2-carboxamide hydro- chloride | ¹H NMR (400 MHz, DMSO- d₆) 8.83 (1H, dd, J = 1.5, 4.7 Hz), 8.43 (1H, d, J = 0.9 Hz), 8.11 (1H, dd, J = 1.5, 8.0 Hz), 7.92-7.89 (1H, m), 7.74-7.72 (2H, m), 7.69 (1H, dd, J = 4.8, 8.0 Hz), 7.63-7.56 (2H, m), 7.49-7.43 (1H, m), 7.35- 7.27 (2H, m), 7.15-7.11 (1H, m), 4.88-4.82 (1H, m), 3.26 (1H, dd, J = 5.1, 14.3 Hz), | (Method 2) RT 2.34 min m/z 359 [MH⁺] |
| | | | 3.19-3.11 (1H, m); | |
| 10 | 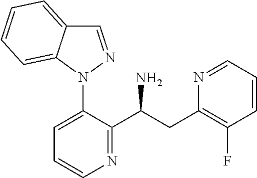 [SMILES: N[C@@H](Cc1ncccc1F)c1ncccc1-n1ncc2ccccc21] | (S)-1-[3-(1H-indazole- 1-yl)pyridine-2-yl]-2- (3-fluoropyridine-2- yl)ethan-1-amine hydrochloride | ¹H NMR (400 MHz, DMSO- d₆) 8.82 (1H, dd, J = 1.4, 4.7 Hz), 8.73 (3H, br s), 8.40 (1H, d, J = 0.7 Hz), 8.06 (1H, dd, J = 1.5, 8.1 Hz), 7.91-7.88 (1H, m), 7.81-7.79 (1H, m), 7.69 (1H, dd, J = 4.7, 8.1 Hz), 7.46-7.42 (1H, m), 7.38- 7.33 (1H, m), 7.31-7.24 (2H, m), 7.06-7.01 (1H, m), 5.25- 5.18 (1H, m), 3.39-3.31 (1H, | (Method 1) RT 2.76 min m/z 334 [MH⁺] |
| | | | m), 3.30-3.23 (1H, m); | |
| 11 | 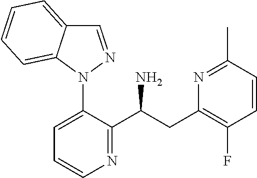 [SMILES: Cc1ccc(F)c(C[C@H](N)c2ncccc2-n2ncc3ccccc32)n1] | (S)-1-[3-(1H-indazole- 1-yl)pyridine-2-yl]-2- (3-fluoro-6-methyl- pyridine-2-yl)ethan-1- amine hydrochloride | ¹H NMR (400 MHz, DMSO- d₆) 8.82 (1H, dd, J = 1.3, 5.0 Hz), 8.76 (3H, br s), 8.41 (1H, d, J = 0.9 Hz), 8.03 (1H, dd, J = 1.5, 8.1 Hz), 7.90-7.87 (1H, m), 7.68 (1H, dd, J = 4.7, 8.1 Hz), 7.44-7.39 (1H, m), 7.31-7.26 (1H, m), 7.22-7.17 (2H, m), 6.75 (1H, dd, J = 3.8, 8.5 Hz), 5.31-5.24 (1H, m), 3.35-3.27 (1H, m), 3.16- | (Method 1) RT 2.87 min m/z 348 [MH⁺] |
| | | | 3.08 (1H, m), 1.94 (3H, s); | |
| 12 | 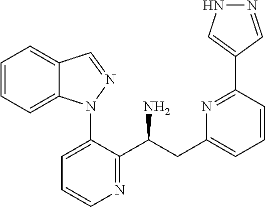 [SMILES: N[C@@H](Cc1cccc(-c2cn[nH]c2)n1)c1ncccc1-n1ncc2ccccc21] | (S)-1-[3-(1H-indazole- 1-yl)pyridine-2-yl]-2- [6-(1H-pyrazol-4-yl)- pyridine-2-yl)ethan-1- amine hydrochloride | ¹H NMR (400 MHz, DMSO- d₆ + TFA) 8.92 (1H, dd, J = 1.3, 4.7 Hz), 8.49 (2H, s), 8.28 (1H, d, J = 0.7 Hz), 8.09 (1H, dd, J = 1.4, 8.0 Hz), 8.01 (1H, t, J = 8.0 Hz), 7.81 (1H, d, J = 8.1 Hz), 7.76-7.72 (2H, m), 7.41-7.36 (1H, m), 7.29- 7.25 (1H, m), 7.25-7.21 (1H, m), 7.10-7.06 (1H, m), 5.41- 5.37 (1H, m), 3.81-3.76 (1H, m), 3.59-3.51 (1H, m); | (Method 1) RT 2.55 min m/z 382 [MH⁺] |
| 13 | 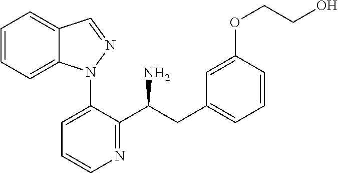 [SMILES: N[C@@H](Cc1cccc(OCCO)c1)c1ncccc1-n1ncc2ccccc21] | (S)-2-[(6-{2-[3-(1H- Indazol-1-yl)pyridine- 2-yl]-2-aminoethyl}- pyridine-2-yl)oxy]- ethan-1-ol hydro- chloride | ¹H NMR (400 MHz, DMSO- d₆) 8.48 (1H, dd, J = 1.3, 4.6 Hz), 8.40 (2H, s), 8.25 (1H, s), 7.65 (1H, dd, J = 1.4, 7.7 Hz), 7.59 (1H, dd, J = 1.4, 8.1 Hz), 7.49 (1H, dd, J = 7.3, 8.3 Hz), 7.43 (1H, dd, J = 4.5, 8.1 Hz), 7.39-7.35 (1H, m), 6.96- 6.92 (1H, m), 6.63 (1H, d, J = 7.1 Hz), 6.55 (1H, d, J = 8.3 Hz), 6.45 (1H, d, J = 8.3 Hz), | (Method 1) RT 2.69 min m/z 376 [MH⁺] |
| | | | 5.34-5.16 (1H, m), 4.12-4.06 | |
| | | | (1H, m), 3.91 (1H, dt, J = 4.8, | |
| | | | 11.3 Hz), 3.59 (2H, t, J = 5.0 | |
| | | | Hz), 3.26 (1H, dd, J = 6.4, | |
| | | | 14.0 Hz), 3.14 (1H, dd, J = | |
| | | | 7.1, 14.0 Hz); | |
| 14 | 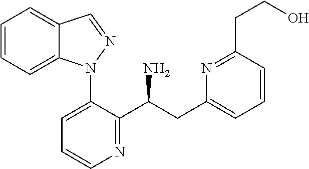 [SMILES: N[C@@H](Cc1cccc(CCO)n1)c1ncccc1-n1ncc2ccccc21] | (S)-2-(6-{2-[3-(1H- Indazol-1-yl)pyridine- 2-yl]-2-aminoethyl}- pyridine-2-yl)ethan-1- ol hydrochloride | ¹H NMR (400 MHz, DMSO- d₆ + TFA) 8.90 (1H, dd, J = 1.4, 4.7 Hz), 8.40 (1H, s), 8.09-8.05 (2H, m), 7.91-7.89 (1H, m), 7.74 (1H, dd, J = 4.7, 8.1 Hz), 7.53 (1H, d, J = 7.9 Hz), 7.48-7.44 (1H, m), 7.33-7.28 (3H, m), 5.35-5.31 (1H, m), 3.76-3.65 (3H, m), 3.57 (1H, dd, J = 9.3, 13.8 Hz), 2.96-2.84 (2H, m); | (Method 1) RT 2.27 min m/z 360 [MH⁺] |
| 15 | 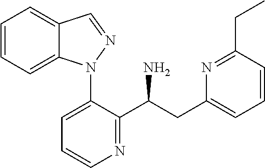 [SMILES: CCc1cccc(C[C@H](N)c2ncccc2-n2ncc3ccccc32)n1] | (S)-1-[3-(1H-indazole- 1-yl)pyridine-2-yl]-2- (6-ethylpyridine-2-yl)- ethan-1-amine hydro- chloride | ¹H NMR (400 MHz, DMSO- d₆ + TFA) 8.90 (1H, dd, J = 1.4, 4.7 Hz), 8.39 (1H, s), 8.10-8.06 (2H, m), 7.89 (1H, d, J = 8.0 Hz), 7.74 (1H, dd, J = 4.7, 8.1 Hz), 7.49-7.43 (2H, m), 7.33-7.25 (3H, m), 5.34-5.30 (1H, m), 3.72 (1H, dd, J = 5.2, 13.8 Hz), 3.56 (1H, dd, J = 9.3, 13.7 Hz), 2.85-2.71 (2H, m), 1.18 (3H, | (Method 1) RT 2.59 min m/z 344 [MH⁺] |
| 16 | 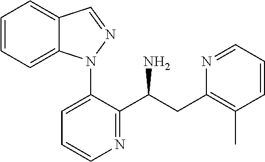 [SMILES: Cc1cccnc1C[C@H](N)c1ncccc1-n1ncc2ccccc21] | (S)-1-[3-(1H-indazole- 1-yl)pyridine-2-yl]-2- (3-methylpyridine-2- yl)ethan-1-amine hydrochloride | ¹H NMR (400 MHz, DMSO- d₆ + TFA) 8.93 (1H, dd, J = 1.4, 4.7 Hz), 8.32-8.30 (1H, m), 8.29 (1H, s), 8.06 (1H, dd, J = 1.4, 8.1 Hz), 7.97 (1H, d, J = 7.9 Hz), 7.87 (1H, d, J = 8.0 Hz), 7.76 (1H, dd, J = 4.7, 8.1 Hz), 7.46-7.41 (1H, m), 7.39 (1H, dd, J = 5.7, 7.9 Hz), 7.32-7.26 (2H, m), 5.40- | (Method 1) RT 2.47 min m/z 330 [MH⁺] |
| | | | 5.36 (1H, m), 3.67 (1H, dd, | |
| | | | J = 4.5, 13.9 Hz), 3.52 (1H, | |
| | | | dd, J = 10.5, 13.9 Hz), 2.04 | |
| | | | (3H, s); | |
| 17 | 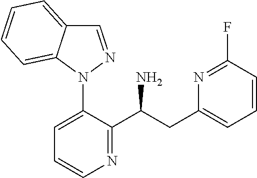 [SMILES: N[C@@H](Cc1cccc(F)n1)c1ncccc1-n1ncc2ccccc21] | (S)-1-[3-(1H-indazole- 1-yl)pyridine-2-yl]-2- (6-fluoropyridine-2- yl)ethan-1-amine hydrochloride | ¹H NMR (400 MHz, DMSO- d₆) 8.84 (1H, dd, J = 1.4, 4.7 Hz), 8.65 (3H, br s), 8.43 (1H, s), 8.06 (1H, dd, J = 1.5, 8.1 Hz), 7.90 (1H, d, J = 8.0 Hz), 7.70 (1H, dd, J = 4.7, 8.1 Hz), 7.61 (1H, q, J = 8.0 Hz), 7.44- 7.40 (1H, m), 7.30-7.27 (1H, m), 7.22 (1H, d, J = 8.5 Hz), 6.79 (1H, dd, J = 2.3, 7.3 Hz), 6.66 (1H, dd, J = 2.3, 8.2 Hz), | (Method 1) RT 2.83 min m/z 334 [MH⁺] |
| | | | 5.13-5.06 (1H, m), 3.15 (2H, | |
| | | | m); | |
| 18 | 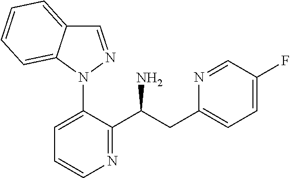 [SMILES: N[C@@H](Cc1ccc(F)cn1)c1ncccc1-n1ncc2ccccc21] | (S)-1-[3-(1H-indazole- 1-yl)pyridine-2-yl]-2- (5-fluoropyridine-2-yl)- ethan-1-amine hydro- chloride | ¹H NMR (400 MHz, DMSO- d₆) 8.84 (1H, dd, J = 1.4, 4.7 Hz), 8.64 (3H, s), 8.44 (1H, s), 8.06 (1H, dd, J = 1.4, 8.1 Hz), 7.91 (1H, d, J = 8.1 Hz), 7.84 (1H, d, J = 3.1 Hz), 7.69 (1H, dd, J = 4.7, 8.1 Hz), 7.45-7.39 (1H, m), 7.36-7.26 (2H, m), 7.21-7.18 (1H, m), 6.90 (1H, dd, J = 4.5, 8.6 Hz), 5.16-5.11 (1H, m), 3.21-3.17 | (Method 1) RT 2.71 min m/z 334 [MH⁺] |
| | | | (2H, m); | |
| 19 | 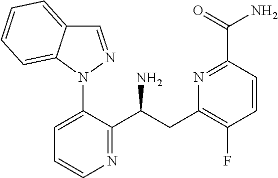 [SMILES: NC(=O)c1ccc(F)c(C[C@H](N)c2ncccc2-n2ncc3ccccc32)n1] | (S)-6-{2-[3-(1H-ind- azol-1-yl)pyridine-2- yl]-2-aminoethyl}-5- fluoropyridine-2- carboxamide hydro- chloride | ¹H NMR (400 MHz, DMSO- d₆) 8.83 (1H, dd, J = 1.4, 4.7 Hz), 8.58 (3H, br s), 8.40 (1H, d, J = 0.8 Hz), 8.15 (1H, dd, J = 1.5, 8.1 Hz), 7.89 (1H, d, J = 8.1 Hz), 7.79 (1H, dd, J = 4.1, 8.6 Hz), 7.73 (1H, dd, J = 4.7, 8.1 Hz), 7.63- 7.59 (1H, m), 7.48-7.44 (1H, m), 7.37 (1H, d, J = 8.6 Hz), 7.32-7.28 (1H, m), 5.16-5.09 | (Method 1) RT 2.42 min m/z 377 [MH⁺] |
| | | | (1H, m), 3.37 (1H, dd, J = | |
| | | | 5.5, 14.7 Hz), 3.27 (1H, dd, | |
| | | | J = 7.7, 14.8 Hz); | |
| 20 | 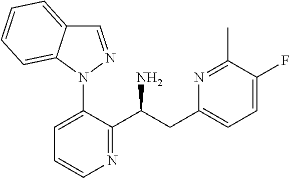 [SMILES: Cc1nc(C[C@H](N)c2ncccc2-n2ncc3ccccc32)ccc1F] | (S)-1-[3-(1H-indazole- 1-yl)pyridine-2-yl]-2- (5-fluoro-6-methyl- pyridine-2-yl)ethan-1- amine hydrochloride | ¹H NMR (400 MHz, DMSO- d₆) 8.85 (1H, dd, J = 1.4, 4.7 Hz), 8.70 (3H, br s), 8.43 (1H, d, J = 0.8 Hz), 8.00 (1H, dd, J = 1.5, 8.1 Hz), 7.89 (1H, d, J = 8.0 Hz), 7.67 (1H, dd, J = 4.7, 8.1 Hz), 7.41-7.37 (1H, m), 7.30-7.26 (1H, m), 7.16 (1H, dd, J = 8.5, 9.6 Hz), 7.08 (1H, d, J = 8.5 Hz), 6.60 (1H, dd, J = 3.6, 8.3 Hz), | (Method 1) RT 2.86 min m/z 348 [MH⁺] |
| | | | 5.24-5.16 (1H, m), 3.15-3.05 | |
| | | | (2H, m), 1.86 (3H, d, J = 2.9 | |
| | | | Hz); | |
| 21 | 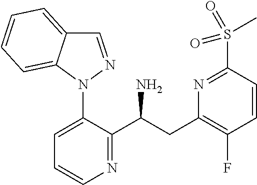 [SMILES: CS(=O)(=O)c1ccc(F)c(C[C@H](N)c2ncccc2-n2ncc3ccccc32)n1] | (S)-1-[3-(1H-indazole- 1-yl)pyridine-2-yl]-2- (3-fluoro-6-methyl- sulfonylpyridine-2-yl)- ethan-1-amine formate | ¹H NMR (400 MHz, DMSO- d₆ + TFA) 8.87 (1H, dd, J = 1.3, 4.7 Hz), 8.38 (1H, s), 8.14-8.12 (1H, m), 7.91 (1H, d, J = 8.0 Hz), 7.80 (1H, dd, J = 3.7, 8.6 Hz), 7.75-7.68 (2H, m), 7.49-7.45 (1H, m), 7.38 (1H, d, J = 8.4 Hz), 7.33- 7.29 (1H, m), 5.20-5.15 (1H, m), 3.43 (2H, d, J = 6.7 Hz), 3.01 (3H, s); | (Method 1) RT 2.59 min m/z 412 [MH⁺] |
| 22 | 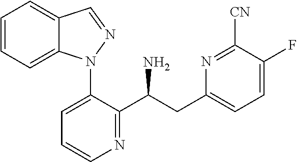 [SMILES: N#Cc1nc(C[C@H](N)c2ncccc2-n2ncc3ccccc32)ccc1F] | (S)-6-{2-[3-(1H-ind- azol-1-yl)pyridine-2- yl]-2-aminoethyl}-3- fluoropyridine-2-carbonitrile hydro- chloride | ¹H NMR (400 MHz, DMSO- d₆) 8.87 (1H, dd, J = 1.5, 4.7 Hz), 8.77 (3H, br s), 8.43 (1H, d, J = 0.9 Hz), 8.04 (1H, dd, J = 1.5, 8.1 Hz), 7.90 (1H, d, J = 8.1 Hz), 7.73-7.65 (2H, m), 7.43-7.39 (1H, m), 7.32- 7.25 (2H, m), 7.18 (1H, d, J = 8.5 Hz), 5.23-5.16 (1H, m), 3.30 (1H, dd, J = 5.4, | (Method 2) RT 2.83 min m/z 359 [MH⁺] |
| | | | 13.5 Hz), 3.21 (1H, dd, J = | |
| | | | 8.8, 13.5 Hz); | |
| 23 | 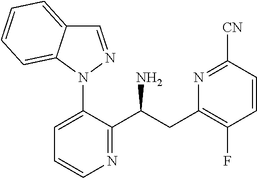 [SMILES: N#Cc1ccc(F)c(C[C@H](N)c2ncccc2-n2ncc3ccccc32)n1] | (S)-6-{2-[3-(1H-ind- azol-1-yl)pyridine-2- yl]-2-aminoethyl}-5- fluoropyridine-2- carbonitrile formate | ¹H NMR (400 MHz, DMSO- d₆) 8.77 (1H, dd, J = 1.5, 4.7 Hz), 8.35 (1H, d, J = 0.8 Hz), 8.22 (1H, s), 7.90 (1H, d, J = 8.1 Hz), 7.86 (1H, dd, J = 1.5, 8.0 Hz), 7.79 (1H, dd, J = 3.8, 8.5 Hz), 7.68 (1H, t, J = 8.8 Hz), 7.53 (1H, dd, J = 4.7, 8.0 Hz), 7.44-7.40 (1H, m), 7.29-7.23 (2H, m), 4.33 (1H, t, J = 7.1 Hz), 3.22-3.16 (1H, | (Method 2) RT 2.73 min m/z 359 [MH⁺] |
| | | | m), 3.11-3.05 (1H, m); | |
| 24 | 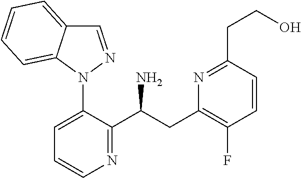 [SMILES: N[C@@H](Cc1nc(CCO)ccc1F)c1ncccc1-n1ncc2ccccc21] | (S)-2-(6-{2-[3-(1H- Indazol-1-yl)pyridine- 2-yl]-2-aminoethyl}-5- fluoropyridine-2-yl)- ethan-1-ol hydrochloride | ¹H NMR (400 MHz, DMSO- d₆) 8.82 (1H, dd, J = 1.4, 4.7 Hz), 8.73 (3H, br s), 8.40 (1H, d, J = 0.8 Hz), 8.05 (1H, dd, J = 1.5, 8.1 Hz), 7.89 (1H, d, J = 8.1 Hz), 7.69 (1H, dd, J = 4.7, 8.0 Hz), 7.45-7.40 (1H, m), 7.30-7.20 (3H, m), 6.87 (1H, dd, J = 3.8, 8.5 Hz), 5.25-5.18 (1H, m), 3.42 (2H, t, J = 6.7 Hz), 3.33-3.27 (1H, | (Method 2) RT 2.67 min m/z 378 [MH⁺] |
| | | | m), 3.19-3.13 (1H, m), 2.47- | |
| | | | 2.36 (2H, m); | |
| 25 | 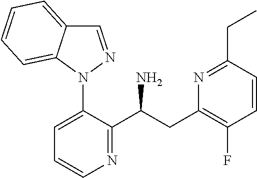 [SMILES: CCc1ccc(F)c(C[C@H](N)c2ncccc2-n2ncc3ccccc32)n1] | (S)-1-[3-(1H-indazole- 1-yl)pyridine-2-yl]-2- (3-fluoro-6-ethylpyr- idine-2-yl)ethan-1- amine hydrochloride | ¹H NMR (400 MHz, DMSO- d₆) 8.82 (1H, dd, J = 1.4, 4.7 Hz), 8.74 (3H, br s), 8.40 (1H, d, J = 0.9 Hz), 8.04 (1H, dd, J = 1.5, 8.1 Hz), 7.88 (1H, d, J = 8.0 Hz), 7.68 (1H, dd, J = 4.7, 8.1 Hz), 7.44-7.40 (1H, m), 7.30-7.19 (3H, m), 6.82 (1H, dd, J = 3.8, 8.5 Hz), 5.29-5.22 (1H, m), 3.36-3.30 (1H, m), 3.17-3.12 (1H, m), 2.32-2.18 (2H, m), 0.88 (3H, t, J = 7.6 Hz); | (Method 2) RT 3.19 min m/z 362 [MH⁺] |
| 26 | 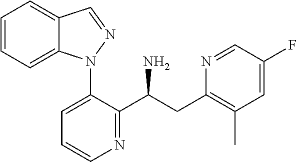 [SMILES: Cc1cc(F)cnc1C[C@H](N)c1ncccc1-n1ncc2ccccc21] | (S)-1-[3-(1H-indazole- 1-yl)pyridine-2-yl]-2- (5-fluoro-3-methyl- pyridine-2-yl)ethan-1- amine hydrochloride | ¹H NMR (400 MHz, DMSO- d₆) 8.84 (1H, dd, J = 1.4, 4.7 Hz), 8.71 (3H, br s), 8.40 (1H, s), 8.05 (1H, dd, J = 1.4, 8.0 Hz), 7.88 (1H, d, J = 8.0 Hz), 7.69 (1H, dd, J = 4.7, 8.1 Hz), 7.66 (1H, d, J = 2.8 Hz), 7.44- 7.40 (1H, m), 7.30-7.26 (1H, m), 7.23-7.17 (2H, m), 5.27- 5.19 (1H, m), 3.20 (2H, m), | (Method 1) RT 2.88 min m/z 348 [MH⁺] |
| | | | 1.88 (3H, s); | |
| 27 | 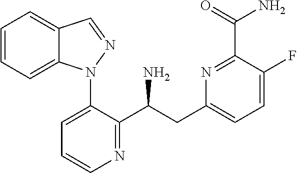 [SMILES: NC(=O)c1nc(C[C@H](N)c2ncccc2-n2ncc3ccccc32)ccc1F] | (S)-6-{2-[3-(1H-ind- azol-1-yl)pyridine-2- yl]-2-aminoethyl}-3- fluoropyridine-2- carboxamide hydro- chloride | ¹H NMR (400 MHz, DMSO- d₆) 8.84 (1H, dd, J = 1.4, 4.7 Hz), 8.53 (3H, br s), 8.44 (1H, d, J = 0.9 Hz), 8.15 (1H, dd, J = 1.4, 8.0 Hz), 7.91 (1H, d, J = 8.1 Hz), 7.73 (1H, dd, J = 4.7, 8.1 Hz), 7.62 (2H, s), 7.56 (1H, dd, J = 8.6, 10.8 Hz), 7.49-7.45 (1H, m), 7.39 (1H, d, J = 8.5 Hz), 7.32- 7.28 (1H, m), 7.17 (1H, dd, | (Method 1) RT 2.24 min m/z 377 [MH⁺] |
| | | | J = 3.4, 8.6 Hz), 5.01-4.93 | |
| | | | (1H, m), 3.28 (1H, dd, J = | |
| | | | 5.4, 14.3 Hz), 3.20 (1H, dd, | |
| | | | J = 7.9, 14.3 Hz); | |
| 28 | 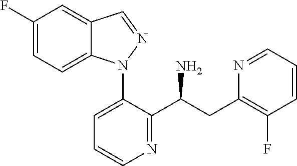 [SMILES: N[C@@H](Cc1ncccc1F)c1ncccc1-n1ncc2cc(F)ccc21] | (S)-1-[3-(5-Fluoro-1H- indazole-1-yl)pyridine- 2-yl]-2-(3-fluoropyr- -idine-2-yl)ethan-1- amine hydrochloride | ¹H NMR (400 MHz, DMSO- d₆) 8.83 (1H, dd, J = 1.4, 4.8 Hz), 8.78 (3H, br s), 8.38 (1H, s), 8.06 (1H, dd, J = 1.5, 8.1 Hz), 7.82-7.80 (1H, m), 7.70- 7.66 (2H, m), 7.39-7.24 (3H, m), 7.08-7.03 (1H, m), 5.23- 5.17 (1H, m), 3.38-3.25 (2H, m); | (Method 1) RT 2.75 min m/z 352 [MH⁺] |
| 29 | 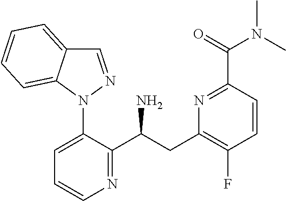 [SMILES: CN(C)C(=O)c1ccc(F)c(C[C@H](N)c2ncccc2-n2ncc3ccccc32)n1] | (S)-6-{2-[3-(1H-ind- azol-1-yl)pyridine-2-yl]- 2-aminoethyl}-5-fluoro- N,N-dimethylpyridine- 2-carboxamide hydro- chloride | ¹H NMR (400 MHz, DMSO- d₆) 8.84 (1H, dd, J = 1.4, 4.7 Hz), 8.69 (3H, br s), 8.36 (1H, s), 8.07 (1H, dd, J = 1.5, 8.1 Hz), 7.92-7.89 (1H, m), 7.70 (1H, dd, J = 4.7, 8.1 Hz), 7.50-7.43 (2H, m), 7.40-7.33 (2H, m), 7.32-7.28 (1H, m), 5.06-5.05 (1H, m), 3.39-3.34 (2H, m), 2.90 (3H, s), 2.56 (3H, s); | (Method 1) RT 2.63 min m/z 4.5 [MH⁺] |
| 30 | 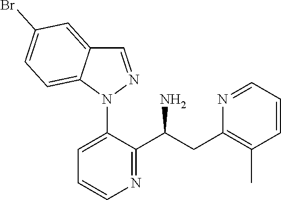 [SMILES: Cc1cccnc1C[C@H](N)c1ncccc1-n1ncc2cc(Br)ccc21] | (S)-1-[3-(5-Bromo-1H- indazole-1-yl)pyridine- 2-yl]-2-(3-methylpyr- idine-2-yl)ethan-1- amine hydrochloride | ¹H NMR (400 MHz, DMSO- d₆) 8.88 (1H, dd, J = 1.4, 4.7 Hz), 8.79 (3H, br s), 8.33 (1H, s), 8.13 (1H, d, J = 1.8 Hz), 8.07 (1H, dd, J = 1.4, 8.1 Hz), 8.03-7.99 (1H, m), 7.72 (1H, dd, J = 4.7, 8.1 Hz), 7.58-7.54 (2H, m), 7.28-7.25 (1H, m), 7.12-7.11 (1H, m), 5.18-5.17 (1H, m), 3.40-3.27 (2H, m), 1.96 (3H, s); | (Method 1) RT 2.87 min m/z 408 [MH⁺] |
| 31 | 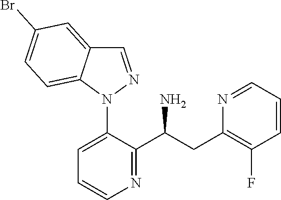 [SMILES: N[C@@H](Cc1ncccc1F)c1ncccc1-n1ncc2cc(Br)ccc21] | (S)-1-[3-(5-Bromo-1H- indazole-1-yl)pyridine- 2-yl]-2-(3-fluoropyr- idine-2-yl)ethan-1- amine hydrochloride | ¹H NMR (400 MHz, DMSO- d₆) 8.83 (1H, dd, J = 1.4, 4.7 Hz), 8.77 (3H, br s), 8.38 (1H, s), 8.15 (1H, d, J = 1.6 Hz), 8.06 (1H, dd, J = 1.4, 8.1 Hz), 7.83-7.81 (1H, m), 7.69 (1H, dd, J = 4.7, 8.1 Hz), 7.57-7.53 (1H, m), 7.41-7.35 (1H, m), 7.26-7.22 (1H, m), 7.08-7.03 (1H, m), 5.18-5.12 (1H, m), 3.38-3.23 (2H, m); | (Method 1) RT 3.03 min m/z 412/414 [MH⁺] |
| 32 | 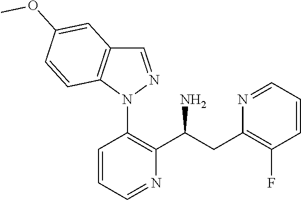 [SMILES: COc1ccc2c(cnn2-c2cccnc2[C@@H](N)Cc2ncccc2F)c1] | (S)-2-(3-fluoropyridine- 2-yl)-1-[3-(5-methoxy- 1H-indazole-1-yl)- pyridine-2-yl]ethan-1- amine hydrochloride | ¹H NMR (400 MHz, DMSO- d₆) 8.79 (1H, dd, J = 1.5, 4.7 Hz), 8.70 (3H, br s), 8.27- 8.27 (1H, m), 8.03 (1H, dd, J = 1.5, 8.1 Hz), 7.86-7.85 (1H, m), 7.67 (1H, dd, J = 4.7, 8.1 Hz), 7.40-7.34 (1H, m), 7.31 (1H, d, J = 2.3 Hz), 7.19-7.15 (1H, m), 7.10-7.04 (2H, m), 5.28-5.21 (1H, m), 3.84 (3H, s), 3.38-3.22 (2H, m); | (Method 2) RT 2.64 min m/z 364 [MH⁺] |
| 33 | 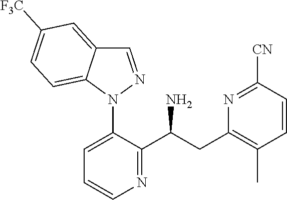 [SMILES: Cc1ccc(C#N)nc1C[C@H](N)c1ncccc1-n1ncc2cc(C(F)(F)F)ccc21] | (S)-6-{2-Amino-2-[3- (5-trifluoromethyl-1H- indazol-1-yl)pyridine- 2-yl]ethyl}-5-methyl- pyridine-2-carbonitrile hydrochloride | ¹H NMR (400 MHz, DMSO- d₆) 8.91 (1H, dd, J = 1.4, 4.7 Hz), 8.64 (3H, br s), 8.57 (1H, d, J = 0.9 Hz), 8.36 (1H, s), 8.12 (1H, dd, J = 1.3, 8.1 Hz), 7.77-7.73 (1H, m), 7.68 (1H, dd, J = 1.7, 9.0 Hz), 7.54-7.51 (1H, m), 7.41-7.33 (2H, m), 5.18-5.12 (1H, m), 3.28-3.18 (2H, m), 1.95 (3H, s); | (Method 1) RT 3.21 min m/z 423 [MH⁺] |
| 34 | 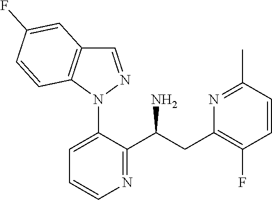 [SMILES: Cc1ccc(F)c(C[C@H](N)c2ncccc2-n2ncc3cc(F)ccc32)n1] | (S)-1-[3-(5-Fluoro-1H- indazole-1-yl)pyridine- 2-yl]-2-(3-fluoro-6- methylpyridine-2-yl)- ethan-1-amine hydro- chloride | ¹H NMR (400 MHz, DMSO- d₆) 8.84 (1H, dd, J = 1.5, 4.7 Hz), 8.78 (3H, br s), 8.39 (1H, d, J = 0.8 Hz), 8.04 (1H, dd, J = 1.5, 8.1 Hz), 7.70-7.66 (2H, m), 7.34-7.28 (1H, m), 7.22-7.16 (2H, m), 6.81-6.76 (1H, m), 5.28-5.21 (1H, m), 3.33-3.24 (1H, m), 3.16-3.09 (1H, m), 1.97 (3H, s); | (Method 1) RT 2.82 min m/z 366 [MH⁺] |
| 35 | 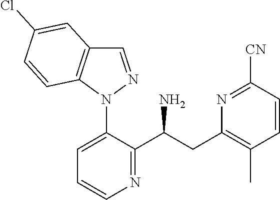 [SMILES: Cc1ccc(C#N)nc1C[C@H](N)c1ncccc1-n1ncc2cc(Cl)ccc21] | (S)-6-{2-Amino-2-[3- (5-chloro-1H-indazol- 1-yl)pyridine-2-yl]- ethyl}-5-methylpyr- idine-2-carbonitrile | ¹H NMR (400 MHz, DMSO- d₆) 8.77 (1H, dd, J = 1.5, 4.7 Hz), 8.33 (1H, d, J = 0.9 Hz), 7.99 (1H, d, J = 1.6 Hz), 7.83 (1H, dd, J = 1.5, 7.9 Hz), 7.55-7.48 (3H, m), 7.40 (1H, dd, J = 2.0, 8.9 Hz), 7.23- 7.20 (1H, m), 4.28 (1H, t, J = 7.0 Hz), 3.19-3.14 (1H, m), 3.00-2.93 (1H, m), 2.03 (3H, s); | (Method 1) RT 3.04 min m/z 389 [MH⁺] |
| 36 | 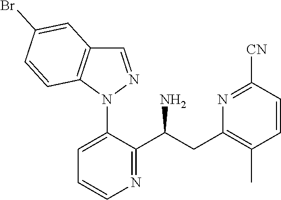 [SMILES: Cc1ccc(C#N)nc1C[C@H](N)c1ncccc1-n1ncc2cc(Br)ccc21] | (S)-6-{2-Amino-2-[3- (5-chloro-1H-indazol- 1-yl)pyridine-2-yl]- ethyl}-5-methylpyr- idine-2-carbonitrile hydrochloride | ¹H NMR (400 MHz, DMSO- d₆) 8.88 (1H, dd, J = 1.4, 4.7 Hz), 8.71 (3H, br s), 8.36 (1H, d, J = 0.8 Hz), 8.12 (1H, d, J = 1.9 Hz), 8.07 (1H, dd, J = 1.5, 8.1 Hz), 7.72 (1H, dd, J = 4.7, 8.1 Hz), 7.55-7.51 (2H, m), 7.42-7.40 (1H, m), 7.22-7.18 (1H, m), 5.22-5.16 (1H, m), 3.29-3.25 (2H, m), 1.95 (3H, s); | (Method 2) RT 3.08 min m/z 433/435 [MH⁺] |
| 37 | 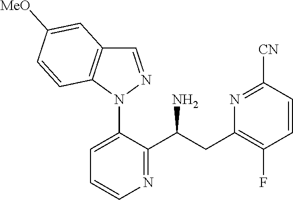 [SMILES: COc1ccc2c(cnn2-c2cccnc2[C@@H](N)Cc2nc(C#N)ccc2F)c1] | (S)-6-{2-Amino-2-[3- (5-methoxy-1H- indazol-1-yl)pyridine- 2-ylethyl}-5-fluoro- pyridine-2-carbonitrile | ¹H NMR (400 MHz, DMSO- d₆) 8.74 (1H, dd, J = 1.5, 4.7 Hz), 8.23 (1H, d, J = 0.8 Hz), 7.84-7.80 (2H, m), 7.70 (1H, t, J = 8.8 Hz), 7.50 (1H, dd, J = 4.7, 7.9 Hz), 7.32 (1H, d, J = 2.2 Hz), 7.16-7.13 (1H, m), 7.05 (1H, dd, J = 2.4, 9.1 Hz), 4.29-4.23 (1H, m), 3.83 (3H, s), 3.20-3.12 (1H, m), 3.10-3.02 (1H, m); | (Method 2) RT 2.66 min m/z 389 [MH⁺] |
| 38 | 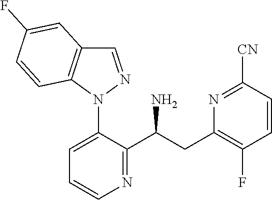 [SMILES: N#Cc1ccc(F)c(C[C@H](N)c2ncccc2-n2ncc3cc(F)ccc32)n1] | (S)-6-{2-Amino-2-[3- (5-fluoro-1H-indazol-1- yl)pyridine-2-yl]ethyl}- 5-fluoropyridine-2- carbonitrile | ¹H NMR (400 MHz, DMSO- d₆) 8.77 (1H, dd, J = 1.5, 4.7 Hz), 8.34 (1H, d, J = 0.9 Hz), 7.87-7.81 (2H, m), 7.73-7.67 (2H, m), 7.53-7.49 (1H, m), 7.34-7.22 (2H, m), 4.24-4.19 (1H, m), 3.22-3.15 (1H, m), 3.09-3.02 (1H, m); | (Method 2) RT 2.70 min m/z 377 [MH⁺] |
| 39 | 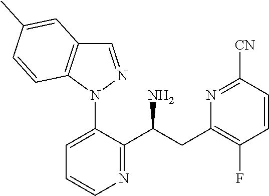 [SMILES: Cc1ccc2c(cnn2-c2cccnc2[C@@H](N)Cc2nc(C#N)ccc2F)c1] | (S)-6-{2-Amino-2-[3- (5-methyl-1H-indazol- 1-yl)pyridine-2-yl]- ethyl}-5-fluoropyr- idine-2-carbonitrile hydrochloride | ¹H NMR (400 MHz, DMSO- d₆) 8.83 (1H, dd, J = 1.4, 4.8 Hz), 8.77 (3H, br s), 8.28 (1H, d, J = 0.7 Hz), 8.04 (1H, dd, J = 1.4, 8.1 Hz), 7.73-7.60 (4H, m), 7.26 (1H, dd, J = 1.3, 8.7 Hz), 7.16-7.12 (1H, m), 5.24-5.17 (1H, m), 3.33-3.26 (2H, m), 2.47 (3H, s); | (Method 1) RT 3.17 min m/z 373 [MH⁺] |
| 40 | 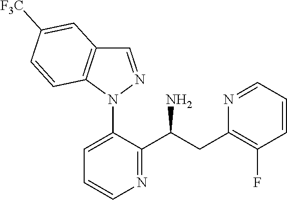 [SMILES: N[C@@H](Cc1ncccc1F)c1ncccc1-n1ncc2cc(C(F)(F)F)ccc21] | (S)-1-[3-(5-Trifluoro- methyl-1H-indazole-1- yl)pyridine-2-yl]-2-(3- fluoropyridine-2-yl)- ethan-1-amine hydro- chloride | ¹H NMR (400 MHz, DMSO- d₆) 8.87 (1H, dd, J = 1.4, 4.7 Hz), 8.73 (3H, br s), 8.58 (1H, d, J = 0.8 Hz), 8.38 (1H, s), 8.12 (1H, dd, J = 1.5, 8.1 Hz), 7.80-7.76 (1H, m), 7.75-7.69 (2H, m), 7.45-7.40 (1H, m), 7.39-7.33 (1H, m), 7.02-6.97 (1H, m), 5.17-5.11 (1H, m), 3.38-3.30 (1H, m), 3.27-3.23 (1H, m); | (Method 1) RT 3.36 min m/z 402 [MH⁺] |
| 41 | 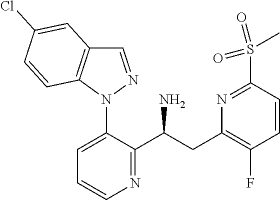 [SMILES: CS(=O)(=O)c1ccc(F)c(C[C@H](N)c2ncccc2-n2ncc3cc(Cl)ccc32)n1] | (S)-1-[3-(5-Chloro- 1H-indazole-1-yl)- pyridine-2-yl]-2-(3- fluoro-6-methylsulfon- ylpyridine-2-yl)ethan- 1-amine hydrochloride | ¹H NMR (400 MHz, DMSO- d₆) 8.87 (1H, dd, J = 1.4, 4.7 Hz), 8.69 (3H, br s), 8.35 (1H, d, J = 0.8 Hz), 8.11 (1H, dd, J = 1.5, 8.1 Hz), 8.00-8.00 (1H, m), 7.82 (1H, dd, J = 3.9, 8.6 Hz), 7.79-7.71 (2H, m), 7.47 (1H, dd, J = 2.0, 8.9 Hz), 7.42-7.39 (1H, m), 5.04 (1H, m), 3.40-3.37 (2H, m), 3.03 (3H, s); | (Method 1) RT 3.09 min m/z 446/448 [MH⁺] |
| 42 | 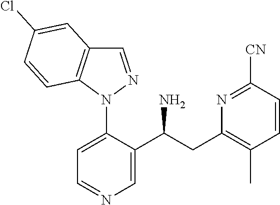 [SMILES: Cc1ccc(C#N)nc1C[C@H](N)c1cnccc1-n1ncc2cc(Cl)ccc21] | (S)-6-{2-Amino-2-[4- (5-chloro-1H-indazol- 1-yl)pyridine-3-yl]- ethyl}-5-methylpyr- idine-2-carbonitrile hydrochloride | ¹H NMR (400 MHz, DMSO- d₆) 9.40-9.39 (1H, m), 8.97 (3H, br s), 8.81 (1H, d, J = 5.4 Hz), 8.48 (1H, d, J = 0.8 Hz), 8.03 (1H, d, J = 1.3 Hz), 7.70 (1H, d, J = 5.4 Hz), 7.61 (1H, d, J = 8.3 Hz), 7.53-7.45 (3H, m), 5.37-5.31 (1H, m), 3.59- 3.41 (2H, m), 2.12 (3H, s); | (Method 1) RT 3.21 min m/z 389/391 [MH⁺] |
| 43 | 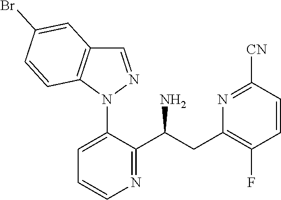 [SMILES: N#Cc1ccc(F)c(C[C@H](N)c2ncccc2-n2ncc3cc(Br)ccc32)n1] | (S)-6-{2-Amino-2-[3- (5-bromo-1H-indazol- 1-yl)pyridine-2-yl- ethyl}-5-fluoropyr- idine-2-carbonitrile hydrochloride | ¹H NMR (400 MHz, DMSO- d₆) 8.87 (1H, dd, J = 1.4, 4.7 Hz), 8.75 (3H, br s), 8.38 (1H, d, J = 0.8 Hz), 8.15 (1H, d, J = 1.5 Hz), 8.08 (1H, dd, J = 1.5, 8.1 Hz), 7.78-7.68 (3H, m), 7.55 (1H, dd, J = 1.9, 8.9 Hz), 7.26-7.22 (1H, m), 5.13- 5.08 (1H, m), 3.31-3.25 (2H, m); | (Method 1) RT 3.22 min m/z 437/439 [MH⁺] |
| 44 | 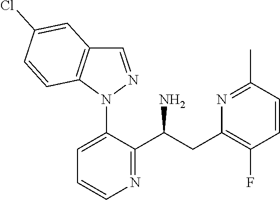 [SMILES: Cc1ccc(F)c(C[C@H](N)c2ncccc2-n2ncc3cc(Cl)ccc32)n1] | (S)-1-[3-(5-Chloro-1H- indazole-1-yl)pyridine- 2-yl]-2-(3-fluoro-6- methylpyridine-2-yl)- ethan-1-amine hydro- chloride | ¹H NMR (400 MHz, DMSO- d₆) 8.85 (1H, dd, J = 1.5, 4.7 Hz), 8.76 (3H, br s), 8.39 (1H, d, J = 0.9 Hz), 8.04 (1H, dd, J = 1.6, 8.1 Hz), 8.00-7.98 (1H, m), 7.71-7.67 (1H, m), 7.42 (1H, dd, J = 2.0, 9.0 Hz), 7.24-7.16 (2H, m), 6.78 (1H, dd, J = 3.8, 8.5 Hz), 5.23-5.18 (1H, m), 3.33-3.25 (1H, m), 3.14-3.08 (1H, m), 1.96 (3H, s); | (Method 1) RT 3.23 min m/z 382/384 [MH⁺] |
| 45 | 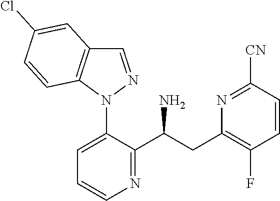 [SMILES: N#Cc1ccc(F)c(C[C@H](N)c2ncccc2-n2ncc3cc(Cl)ccc32)n1] | (S)-6-{2-Amino-2-[3- (5-chloro-1H-indazol- 1-yl)pyridine-2-yl- ethyl}-5-fluoropyr- idine-2-carbonitrile hydrochloride | ¹H NMR (400 MHz, DMSO- d₆) 8.87 (1H, dd, J = 1.3, 4.7 Hz), 8.81 (3H, br s), 8.38 (1H, d, J = 0.9 Hz), 8.07 (1H, dd, J = 1.5, 8.1 Hz), 8.01-7.99 (1H, m), 7.78-7.67 (3H, m), 7.45 (1H, dd, J = 2.0, 8.9 Hz), 7.32-7.28 (1H, m), 5.14-5.07 (1H, m), 3.41-3.27 (2H, m); | (Method 1) RT 3.16 min m/z 393/395 [MH⁺] |
| 46 | 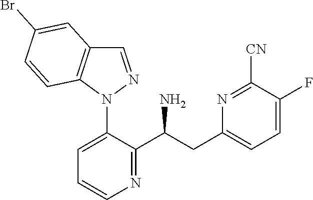 [SMILES: N#Cc1nc(C[C@H](N)c2ncccc2-n2ncc3cc(Br)ccc32)ccc1F] | (S)-6-{2-Amino-2-[3- (5-bromo-1H-indazol- 1-yl)pyridine-2-yl]- ethyl}-3-fluoropyr- idine-2-carbonitrile hydrochloride | ¹H NMR (400 MHz, DMSO- d₆) 8.89 (1H, dd, J = 1.4, 4.7 Hz), 8.80 (3H, br s), 8.42 (1H, d, J = 0.8 Hz), 8.14 (1H, d, J = 1.5 Hz), 8.05 (1H, dd, J = 1.5, 8.1 Hz), 7.74-7.69 (2H, m), 7.52 (1H, dd, J = 1.8, 8.9 Hz), 7.29 (1H, dd, J = 4.2, 8.8 Hz), 7.16-7.13 (1H, m), 5.13- 5.09 (1H, m), 3.33-3.27 (1H, m), 3.24-3.18 (1H, m); | (Method 1) RT 3.30 min m/z 437/439 [MH⁺] |
| 47 | 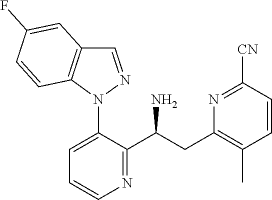 [SMILES: Cc1ccc(C#N)nc1C[C@H](N)c1ncccc1-n1ncc2cc(F)ccc21] | (S)-6-{2-Amino-2-[3- (5-fluoro-1H-indazol-1- yl)pyridine-2-ylethyl}- 3-methylpyridine-2- carbonitrile | ¹H NMR (400 MHz, DMSO- d₆) 8.76 (1H, dd, J = 1.6, 4.7 Hz), 8.33 (1H, d, J = 0.9 Hz), 7.83 (1H, dd, J = 1.6, 8.0 Hz), 7.67 (1H, dd, J = 2.1, 8.9 Hz), 7.55-7.48 (3H, m), 7.31-7.26 (1H, m), 7.22-7.18 (1H, m), 4.31 (1H, t, J = 7.1 Hz), 3.20- 3.14 (1H, m), 3.00-2.94 (1H, m), 2.02 (3H, s); | (Method 1) RT 2.97 min m/z 373 [MH⁺] |
| 48 | 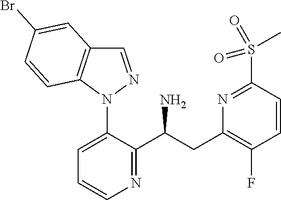 [SMILES: CS(=O)(=O)c1ccc(F)c(C[C@H](N)c2ncccc2-n2ncc3cc(Br)ccc32)n1] | (S)-1-[3-(5-Bromo-1H- indazole-1-yl)pyridine- 2-yl]-2-(3-fluoro-6- methylsulfonylpyr- idine-2-yl)ethan-1- amine hydrochloride | ¹H NMR (400 MHz, DMSO- d₆) 8.68 (3H, br s), 8.30 (1H, s), 8.14-8.09 (2H, m), 7.86 (1H, dd, J = 3.8, 8.5 Hz), 7.81-7.71 (1H, m), 7.64-7.58 (1H, m), 7.52 (1H, dd, J = 1.8, 8.9 Hz), 7.51-7.47 (1H, m), 7.21 (1H, d, J = 8.9 Hz), 4.68 (1H, t, J = 7.1 Hz), 3.53- 3.44 (1H, m), 3.42-3.37 (1H, m), 2.93 (3H, s); | (Method 2) RT 3.15 min m/z 489/491 [MH⁺] |
| 49 | 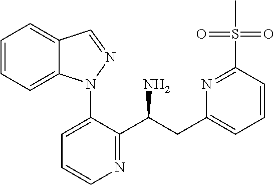 [SMILES: CS(=O)(=O)c1cccc(C[C@H](N)c2ncccc2-n2ncc3ccccc32)n1] | (S)-1-[3-(1H-indazole- 1-yl)pyridine-2-yl]-2- (6-methylsulfonyl- pyridine-2-yl)ethan-1- amine hydrochloride | ¹H NMR (400 MHz, DMSO- d₆) 8.85 (1H, dd, J = 1.4, 4.7 Hz), 8.64 (3H, br s), 8.43 (1H, d, J = 0.9 Hz), 8.12 (1H, dd, J = 1.5, 8.1 Hz), 7.92-7.89 (1H, m), 7.86 (1H, t, J = 7.7 Hz), 7.74-7.69 (2H, m), 7.49- 7.44 (1H, m), 7.40-7.36 (1H, m), 7.32-7.25 (2H, m), 5.07- 5.00 (1H, m), 3.40-3.26 (2H, m), 2.98 (3H, s); | (Method 1) RT 2.53 min m/z 394 [MH⁺] |

### Biological Assays

The biological effects of the compounds may be assessed using one of more of the assays described herein.

### Example 50: Assay for HCN1 HCN2 and HCN4

### Example 50A, Using PatchXpress 7000A

Solutions for recording HON currents were:

| | |
|---|---|
| External Recording Solution | Internal Recording Solution |

| | | | | | |
|---|---|---|---|---|---|
| NaCl | 110 | mM | KCl | 60 | mM |
| KCl | 30 | mM | KF | 70 | mM |
| MgCl2 | 1 | mM | NaCl | 10 | mM |
| CaCl2 | 1.8 | mM | HEPES | 10 | mM |
| HEPES | 10 | mM | EGTA | 11 | mM |
| Glucose | 5 | mM | MgATP | 2 | mM |

| | |
|---|---|
| pH 7.4 (titrated with NaOH) | pH 7.35 (titrated with KOH) |

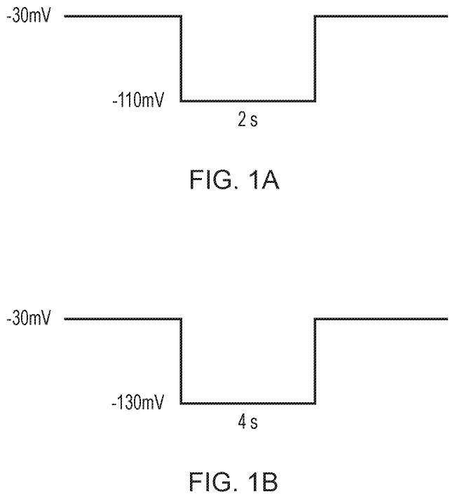

For HCN1 and HCN2, the pulse protocol involved stepping from a holding potential of −30 mV to −110 mV (see FIG. 1A) for 2 seconds to evoke the current. The membrane voltage was then stepped back to −30 mV for a further 8 seconds. This sequence was evoked repeatedly every 10 seconds throughout the experiment, starting prior to drug (Control A) and during cumulative additions of 5 increasing compound concentrations, then finally a 100% inhibiting concentration of cesium chloride (CsCl, 3 mM).

For HCN4, the pulse protocol involved stepping from a holding potential of −30 mV to −130 mV (see FIG. 1B) for 4 seconds to evoke the current. The membrane voltage was then stepped back to −30 mV, the voltage protocol had a start-to-start interval of 14 seconds, starting prior to drug (Control A) and during cumulative additions of increasing compound concentrations, then finally a 100% inhibiting concentration of cesium chloride (CsCl, 3 mM).

The peak inward current measured at the end of the pulse to −110 mV (HCN1 and HCN2) or −130 mV (HCN4) was measured and any leak current subtracted to calculate the HCN current. The HCN current amplitude was measured after each control or compound addition and normalized to the control amplitude (Control A).

All experiments were performed at room temperature (approximately 22° C.).

Each test compound concentration was applied to the cell for seven (7) minutes, at which point the next cumulative concentration was applied. 3 mM CsCl was applied to each cell for 2 minutes at the end of each experiment (Control B) to determine 100% inhibition level of the HCN current.

### Example 50B, Using the Sophion Qube

Solutions for recording HCN currents were:

| External Recording Solution | | | Internal Recording Solution | | |
|---|---|---|---|---|---|
| NaCl | 110 | mM | KCl | 20 | mM |
| KCl | 30 | mM | KF | 110 | mM |
| MgCl2 | 1 | mM | NaCl | 10 | mM |
| CaCl2 | 1.8 | mM | HEPES | 10 | mM |
| HEPES | 10 | mM | EGTA | 11 | mM |
| Glucose | 10 | mM | MgATP | 2 | mM |

| | |
|---|---|
| pH 7.4 (titrated with NaOH) | pH 7.2 (titrated with KOH) |
| | Osmolality adjusted to 290 mOsm |

For HCN1 and HCN2 the cells were held at −30 mV and then stepped to −110 mV for 2 seconds before stepping back to −30 mV, this represents 1 experimental sweep. This voltage protocol was applied every 20 seconds for the duration of the experiment. Both the vehicle (0.3% DMSO) and full block (3 mM CsCl) addition periods were applied for 10 experimental sweeps each. The compound addition period was applied for 30 sweeps.

For HCN4, the cells were held at −30 mV and then stepped to −130 mV for 4 seconds before stepping back to −30 mV, this represents 1 experimental sweep. This voltage protocol was applied every 20 seconds for the duration of the experiment. Both the vehicle (0.3% DMSO) and full block (3 mM CsCl) addition periods were applied for 10 experimental sweeps each. The compound addition period was applied for 30 sweeps.

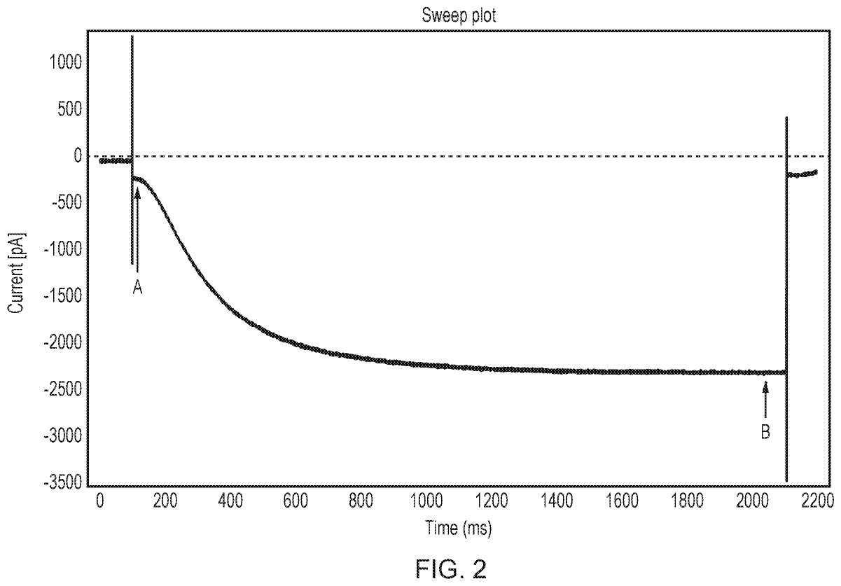

The currents evoked by the step to −110 mV (HCN1 and HCN2) or −130 mV (HCN4) were measured for the analysis of the percentage inhibition by test compounds. The current amplitudes were measured by subtracting metric A from metric B (see FIG. 2) with inhibition calculated by normalising to the vehicle addition (0.3% DMSO) and full inhibition by 3 mM CsCl in the same well.

The potency (IC₅₀) of test compound to inhibit the HCN ion channel was determined from a concentration-response curve generated from up to 8 test compound concentrations with up to 8 replicates per concentration. In total, compound was applied to the well for 600 seconds.

### Example 51: Assay for hERG

### Example 51A, Using IonWorks Quattro

Solutions for recording hERG currents were:

| | |
|---|---|
| External Recording Solution | Internal Recording Solution |

| | | | | | |
|---|---|---|---|---|---|
| NaCl | 138 | mM | KCl | 140 | mM |
| KCl | 2.7 | mM | MgCl₂ | 1 | mM |
| MgCl₂ | 0.5 | mM | HEPES | 20 | mM |
| CaCl₂ | 0.9 | mM | EGTA | 1 | mM |

| | | |
|---|---|---|
| Na₂HPO₄ | 8 | mM |
| KH₂PO₄ | 1.5 | mM |

| | |
|---|---|
| pH 7.3 (titrated with NaOH) | pH 7.3 (titrated with KOH) |

Electrophysiological recordings were made from a Human Embryonic Kidney (HEK) cell line stably expressing the full length hERG channel. Single cell ionic currents were measured in the perforated patch clamp configuration (100 μg ml⁻¹ amphotericin) at room temperature (approx. 22° C.) using an IonWorks Quattro from Molecular Devices.

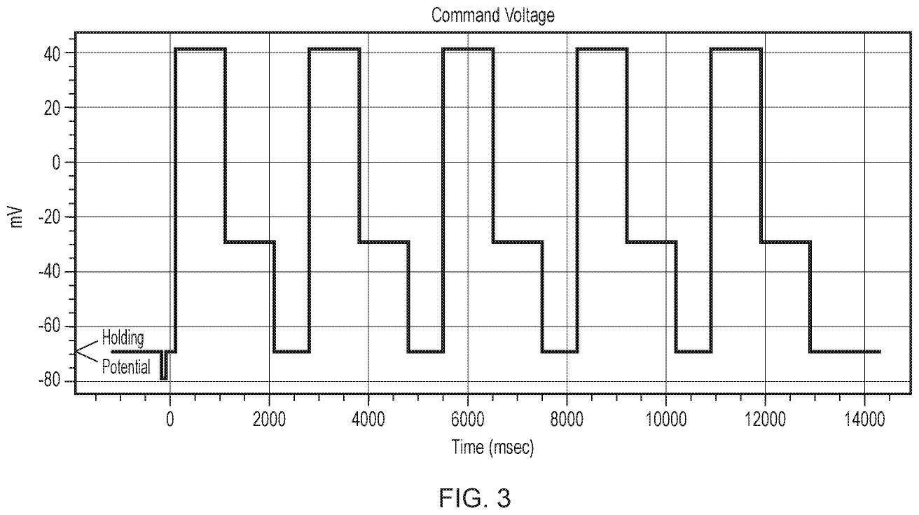

Cells were clamped at a holding potential of −70 mV for 30 s and then stepped to +40 mV for 1 s. This was followed by a hyperpolarising step of 1 s to −30 mV to evoke the hERG tail current. This sequence was repeated 5 times at a frequency of 0.25 Hz (see FIG. 3). Currents were measured from the tail step at the 5th pulse and referenced to the holding current. Compounds were then incubated for 6-7 minutes prior to a second measurement of the hERG signal using an identical pulse train.

The potency (IC₅₀) of test compounds to inhibit the hERG channel were determined from a concentration-response curve generated from up to 8 test compound concentrations with up to 4 replicates per concentration.

### Example 51B, Using Sophion Qube

Solutions for recording hERG currents were:

| | |
|---|---|
| External Recording Solution | Internal Recording Solution |

| | | | | | |
|---|---|---|---|---|---|
| NaCl | 145 | mM | KCl | 20 | mM |
| KCl | 4 | mM | KF | 120 | mM |
| MgCl₂ | 1 | mM | HEPES | 10 | mM |
| CaCl₂ | 2 | mM | EGTA | 10 | mM |
| HEPES | 10 | mM | | | |
| Glucose | 10 | mM | | | |

| | |
|---|---|
| pH 7.4 (titrated with NaOH) | pH 7.2 (titrated with KOH) |

The cells were held at a voltage of −80 mV and then stepped to +40 mV for 2 seconds before stepping to −40 mV for a further 2 seconds, this represents 1 experimental sweep. This voltage protocol was applied every 15 seconds for the duration of the experiment. Both the vehicle and 1st compound addition periods were applied for 10 sweeps. The 2nd compound addition period was applied for 20 sweeps. The compound concentration was added to the test well twice to assure complete exchange of the external buffer with the test compound. In total, compound was applied to the well for 450 seconds.

The peak tail currents evoked by the step to −40 mV were measured for the analysis of the percentage inhibition by test compounds. The peak tail currents were first normalised to the vehicle addition (0.3% DMSO) in the same well.

The potency (IC50) of test compounds to inhibit the hERG channel were determined from a concentration-response curve generated from up to 8 test compound concentrations with up to 4 replicates per concentration.

### Example 52: Assay for hNaᵥ1.5

### Example 52A, Using IonWorks Quattro

Solutions for recording Naᵥ1.5 currents were:

| | |
|---|---|
| External Recording Solution | Internal Recording Solution |

| | | | | | |
|---|---|---|---|---|---|
| NaCl | 137 | mM | K-gluconate | 90 | mM |
| KCl | 4 | mM | KCl | 40 | mM |
| MgCl2 | 1 | mM | NaCl | 10 | mM |
| CaCl2 | 1.8 | mM | MgCl₂ | 3.2 | mM |
| HEPES | 10 | mM | HEPES | 5 | mM |
| | | | EGTA | 3.2 | mM |

| | |
|---|---|
| pH 7.3 (titrated with NaOH) | pH 7.3 (titrated with KOH) |

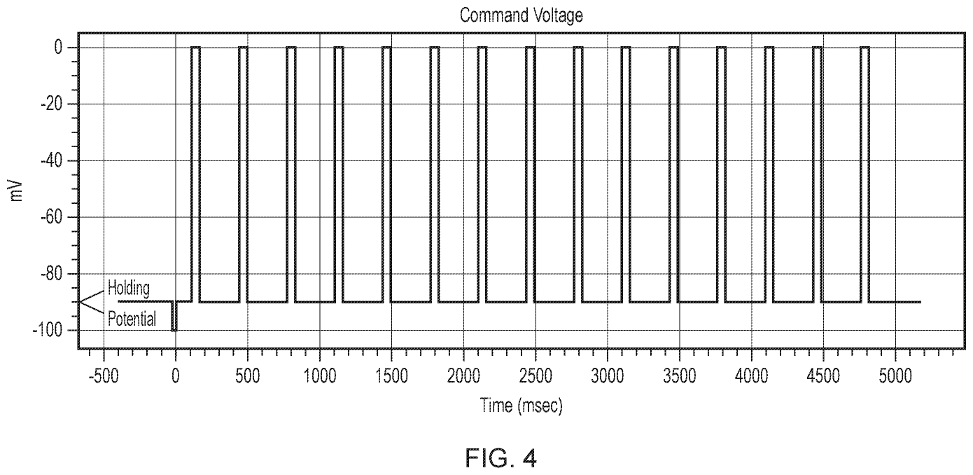

Electrophysiological recordings were made from a human embryonic kidney (HEK) cell line stably expressing the full length hNa_{V}1.5. Population patch clamp measurements were made in the perforated patch clamp configuration (100 μg ml⁻¹ amphotericin) at room temperature (approx. 220C) using an IonWorks Quattro from Molecular Devices. The voltage protocol is illustrated in FIG. 4. Currents were first measured under control (pre-compound addition) conditions. Compounds were then incubated for 5-7 minutes prior to a second measurement of the hNa_{V}1.5 signal using an identical pulse train. Currents were measured from the depolarising step at the 15^{th} pulse and referenced to the holding current.

### Example 52B, Using Sophion Qube

The cells were held at −100 mV followed by a depolarising step to −10 mV for 100 milliseconds before stepping back to −100 mV, this represents 1 experimental sweep. This voltage protocol was applied at 0.1 Hz and 4 Hz, to evaluate both tonic block, and the potential for use-dependent block of the hNaᵥ1.5 channel. The vehicle and compound addition periods were applied for 20 sweeps at 0.1 Hz to assess tonic block, and as a train of 20 depolarisations at a frequency of 4 Hz to test for use-dependent block.

For tonic block (0.1 Hz), the peak currents evoked by the step to −10 mV were measured for the analysis of the percentage inhibition by test compounds. For use-dependent block (4 Hz), the peak current evoked at the 20^{th} depolarising step to −10 mV was measured for the analysis of the percentage inhibition by test compounds. Peak currents were first normalised to the vehicle addition (0.3% DMSO) in the same well. The potency (IC₅₀) of test compounds to inhibit the hNaᵥ1.5 channel were determined from a concentration-response curve generated from up to 8 test compound concentrations with up to 4 replicates per concentration and are quoted for the use dependent block.

### Example 53: MDCK Assay

The bi-directional MDCK permeability assay in MDCK-MDR1 cells was performed using MDCK-MDR1 cells (Solvo Biotechnology) seeded onto 24-well Transwell plates at 2.35×105 cells per well and used in confluent monolayers after a 3 day culture at 37° C. under 5% CO₂. Test compounds were added (10 μM, 0.1% DMSO final, n=2) to donor compartments of the Transwell plate assembly in assay buffer (Hanks balanced salt solution supplemented with 25 mM HEPES, adjusted to pH 7.4) for both apical to basolateral (A>B) and basolateral to apical (B>A) measurements. A parallel series of incubations were performed in the presence of the transporter inhibitor elacridar (5 μM) which was added to both compartments in the transwell plate. Incubations were performed at 37° C., with samples removed from both donor and acceptor chambers at T=0 and 1 hour for recovery assessment and compound analysed by mass spectrometry (LC-MS/MS), including an analytical internal standard.

Apparent permeability (Papp) values were determined from the relationship:

Papp=[CompoundAcceptor T=end]×VAcceptor/([CompoundDonor T=0]×VDonor)/incubation time×VDonor/Area×60×10-6 cm/s

Where V is the volume of each Transwell compartment (apical 125 μL, basolateral 600 μL), and concentrations are the relative MS responses for compound (normalized to internal standard) in the donor chamber before incubation and acceptor chamber at the end of the incubation.

Area=area of cells exposed for drug transfer (0.33 cm2).

Efflux ratios (Papp B>A/Papp A>B) were calculated for each compound from the mean Papp values in each direction. The MDCK-MDR1 cell line has been engineered to over-express the efflux transporter, MDR1 (P-glycoprotein), and a finding of good permeability B>A, but poor permeability A>B, suggests that a compound is a substrate for this transporter. The efflux ratios were also calculated in the same way from the runs carried out in the presence of the inhibitor. The net flux is the ratio of the efflux in the absence of inhibitor to that in the presence of inhibitor. A net flux value >5 (i.e. efflux ratio without inhibitor divided by efflux ratio plus inhibitor) is indicative of compounds being substrates for the transporter P-gp and would therefore have a greater likelihood of being restricted from the CNS (i.e. peripherally restricted).

Lucifer Yellow (LY) was added to the apical buffer in all wells to assess viability of the cell layer. As LY cannot freely permeate lipophilic barriers, a high degree of LY transport indicates poor integrity of the cell layer and wells with a LY Papp >10×10⁻⁶ cm/s were rejected. Note that an integrity failure in one well does not affect the validity of other wells on the plate.

Compound recovery from the wells was determined from MS responses (normalized to internal standard) in donor and acceptor chambers at the end of incubation compared to response in the donor chamber pre-incubation. Recoveries <50% suggest compound solubility, stability or binding issues in the assay which may reduce the reliability of a result.

### Biological Data

Table 4 shows the IC₅₀ values in μM for HCN2 and HCN4 using the PatchXpress protocol (PX) for compounds tested.

TABLE 4

| | PX HCN2 | PX HCN4 |
|---|---|---|
| Compound No | IC₅₀ (μM) | IC₅₀ (μM) |
| 1 | 0.9 | 3.4 |
| 2 | 0.03 | 0.5 |
| 3 | 3.5 | >30 |
| 4 | 1.6 | >30 |
| 5 | 1.9 | >30 |
| 6 | 1.1 | 22.2 |
| 7 | 7.0 | >30 |
| 8 | 28.1 | >30 |
| 9 | 17.3 | >30 |
| 10 | 0.08 | 4.2 |
| 11 | 0.16 | 8.3 |
| 12 | >10 | — |
| 13 | >10 | — |
| 14 | 3.7 | — |
| 15 | 2.1 | 14.8 |
| 16 | 0.13 | 2.1 |
| 17 | 3.3 | 14.7 |
| 18 | 0.5 | 11.7 |
| 19 | 6.9 | — |
| 20 | 0.9 | 13.0 |
| 21 | 1.1 | 30 |
| 22 | 0.32 | 13.4 |
| 23 | 0.18 | 7.5 |
| 24 | 0.9 | 19.2 |
| 25 | 0.34 | 1.6 |
| 26 | 0.015 | 0.5 |
| 27 | >10 | — |
| 28 | 1.0 | >30 |
| 29 | 2.6 | — |
| 49 | ~10 | — |

Table 5 shows the IC₅₀ values in μM for HCN2 and HCN4 using the Sophion Qube protocol (SQ) for the compounds tested.

TABLE 5

| | SQ HCN2 | SQ HCN4 |
|---|---|---|
| Compound No | IC₅₀ (μM) | IC₅₀ (μM) |
| 30 | 0.9 | 22.5 |
| 31 | 0.9 | — |
| 32 | 28.6 | — |
| 33 | 4.9 | — |
| 34 | 4.5 | — |
| 35 | 0.46 | 4.3 |
| 36 | 0.77 | 4.7 |
| 37 | 6.7 | — |
| 38 | 0.79 | >30 |
| 39 | 0.31 | 3.7 |
| 40 | 11.7 | — |
| 41 | 3.9 | >30 |
| 42 | 4.9 | — |
| 43 | 0.78 | — |
| 44 | 1.5 | — |
| 45 | 0.60 | 3.4 |
| 46 | 1.2 | — |
| 47 | 0.63 | 10.0 |
| 48 | 2.1 | 11.1 |

### Example 54: Effect on Tinnitus by Pharmacological Block of HCN2 Ion Channels

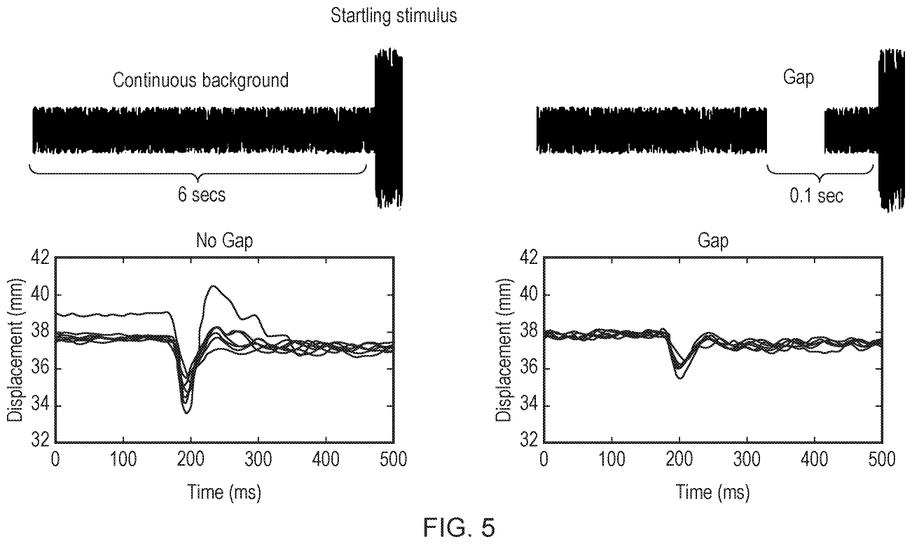

Tinnitus in guinea pigs was monitored using the gap induced inhibition of the acoustic startle (GPIAS) test (see FIG. 5). GPIAS is reduced when tinnitus was present; see Berger, J. I. et al. Effects of the cannabinoid CB1 agonist ACEA on salicylate ototoxicity, hyperacusis and tinnitus in guinea pigs. Hearing research, (2017), and Coomber, B. et al. Neural changes accompanying tinnitus following unilateral acoustic trauma in the guinea pig. Eur J Neurosci 40, 2427-2441, (2014).

In FIG. 5, sound stimulus (above) and corresponding pinna reflex (below) are shown in freely moving guinea pigs. Stimuli with no gap and gap are presented in a randomised order. Traces contaminated by movement (the upper trace in “no gap”) were removed before analysis.

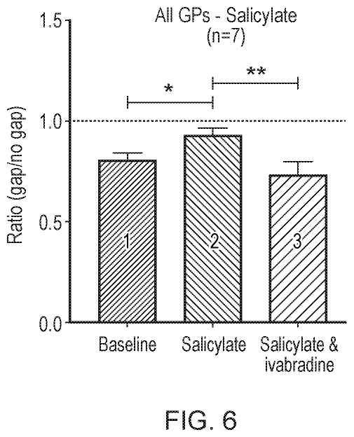

Tinnitus was induced within 1-2 hours in humans by high doses of salicylate. A similar short-term tinnitus model was implemented in guinea pigs by i.p. injection of salicylate. In all animals, salicylate caused behavioural inhibition of GPIAS (see bar 2 in FIG. 6). Block of HCN ion channels by the non-selective inhibitor ivabradine (which blocks HCN1-4 equally) reversed GPIAS (see bar 3 in FIG. 6). Thus, it was found that HCN ion channel block reverses behavioural signs of tinnitus in this short-term (salicylate) model.

Salicylate (350 mg/kg, i.p.) impairs behavioural gap detection 2 h after salicylate administration (see bar 2 in of FIG. 6). Gap detection was restored by blocking HCN channels with ivabradine (5 mg/kg, s.c.).

Mild unilateral noise exposure has been found to reduce GPIAS in around 40% of guinea pigs, an observation that resembles the effect of noise in humans, where noise exposure causes tinnitus in some but not all subjects. The noise-exposure model is more clinically relevant than the salicylate model, as it parallels a common cause of tinnitus in humans. A second important point is that it is long-term, while tinnitus induced by salicylate is rapidly reversed following salicylate exposure.

The reduced GPIAS seen following noise exposure (see bar B in FIG. 7) was found to be rapidly and completely reversed by HCN ion channel block with ivabradine, which blocks all four HCN ion channel isoforms equally (see bar C in FIG. 7). GPIAS returns following drug wash-out (see bar D in FIG. 7). Thus, it was found that block of HCN ion channels abolishes behavioural signs of tinnitus.

A peripherally restricted and HCN2-selective compound (“compound 476” in FIG. 7), chemically unrelated to ivabradine and not within the scope of the claims, also caused a complete reversal of “tinnitus” behaviour (see bar E in FIG. 7). In control experiments on noise-exposed guinea pigs showing no behavioural evidence of tinnitus, ivabradine was without effect on GPIAS (n=3, results not shown). The similar results obtained in the short-term salicylate model and in the long-term noise-exposure model suggest that tinnitus is both initiated and maintained by activity of HCN2 ion channels.
Bar A: naïve guinea pigs showed a large reduction in the acoustic startle response following a brief gap in continuous noise.
Bar B: following unilateral noise exposure (NE, 110 dB, 1 h, 8 weeks prior to testing) around 40% of guinea pigs developed impaired GPIAS.
Bar C: non-selective HCN inhibitor ivabradine (5 mg/kg, s.c.) fully restores GPIAS. Dark grey bar: reduced GPIAS returns following drug washout (1-2 d).
Bar E: a compound with high selectivity for HCN2 over HCN1 (28x) and HCN4 (63x) fully restored GPIAS at the same dose that achieves full block of neuropathic pain (0.5 mg/kg, s.c.).

In control experiments on noise-exposed guinea pigs showing no behavioural tinnitus, ivabradine was without effect on gap detection (not shown).

### Example 55: Evaluation of CNS Penetration of HCN Blocker Ivabradine

Ivabradine in guinea pig plasma, brain (somatosensory cortex) and auditory nerve were assayed at 30 min after injection, the time used in Example 130.

Ratios of total concentrations in preliminary experiments for plasma:brain:auditory nerve were 1:0.12:0.57 (n=2). The small amount (12% of plasma level) detected in brain is largely accounted for by the presence of ivabradine within the vascular supply of the brain. As in other species, therefore, ivabradine is strongly excluded from guinea pig brain because of its hydrophilicity and Pgp substrate activity; see Young, G. T., Emery, E. C., Mooney, E. R., Tsantoulas, C. & McNaughton, P. A. Inflammatory and neuropathic pain are rapidly suppressed by peripheral block of hyperpolarisation-activated cyclic nucleotide-gated ion channels. Pain 155, 1708-1719, (2014).

The ratio of 0.57 between auditory nerve and plasma total concentrations shows that ivabradine is not excluded from auditory nerve, which is therefore accessible to plasma concentrations of ivabradine. The difference from a value of 1 may be accounted for by differences in binding to proteins in plasma and auditory nerve. Thus, it was found that the HCN blocker ivabradine penetrates the auditory nerve but not the CNS.

### Example 56: Effect of Genetic Deletion or Pharmacological Block of HCN2 on Hearing Thresholds

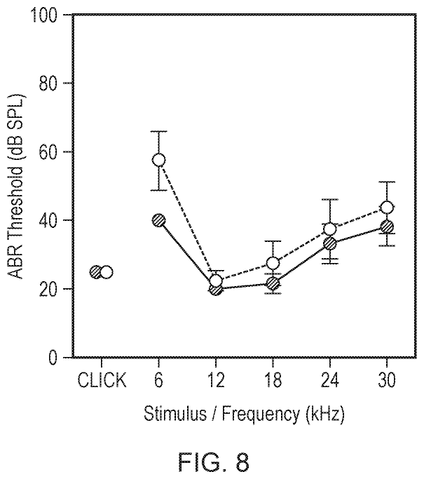

In this example the effect of genetic deletion or pharmacological block of HCN2 on auditory brainstem response (ABR) thresholds to tone pulses, with frequencies from 3 kHz to 42 kHz was assed. Results are shown in FIG. 8, in which WT mice and sox10-Cre^{+/-}/fHCN2 litter mates (auditory-targeted HCN2 deletion) show no significant difference in ABR threshold or latency. Similar results were obtained in adult mice treated with the non-selective HCN blocker ivabradine and with a chemically unrelated HCN2-selective blocker (20 mg/kg ip). No significant difference in hearing in mice with a global genetic deletion of HCN2 was found, but in this case the hearing was compared with WT littermates at age 2 weeks as the HCN2^{-/-} mice die by 3-4 weeks.

Mice carrying an auditory-targeted HCN2 deletion (upper line of unfilled dots in FIG. 8) and WT littermates (lower line of filled dots in FIG. 8) show no significant difference in either threshold (FIG. 8) or response latency (data not shown, latency of P1 and N1 waves measured with click and at 12 KHz and 18 kHz). Deletion of HCN2 expressed in spiral ganglion neurons therefore does not affect normal hearing thresholds or response latencies. Bars show SD (n=6).

These results indicate that HCN2 does not participate in normal hearing and is only activated in pathological circumstances, such as following noise exposure.

### Example 57: Mechanical Analgesic Effects of Compounds in a Mouse Neuropathic Pain Model Tested Using a Von Frey Filament

The compounds of Example 2 and Example 4 were tested in a mouse neuropathic pain model using WT Black6 strain mice. The model used was analogous to the model described in Seltzer Z, Dubner R, & Shir Y (1990), A novel behavioural model of neuropathic pain disorders produced in rats by partial sciatic nerve injury, Pain 43: 205-218). Further details of the experimental procedures used are described in Young G T et al. (2014), Inflammatory and neuropathic pain are rapidly suppressed by peripheral block of hyperpolarisation-activated cyclic nucleotide-gated ion channels; Pain 155: 1708-1719; and Tsantoulas C et al., (2017), Hyperpolarization-activated cyclic nucleotide-gated 2 (HCN2) ion channels drive pain in mouse models of diabetic neuropathy. Sci Transl Med 9: eaam6072.

The test compounds were administered i.p. to the mice on day 5 following partial sciatic nerve ligation surgery, average data from 3-4 mice. The mechanical pain threshold was measured by manual von Frey filament applied to hind paw on the operated side, using the “up-down” method. The test compounds were compared to ivabradine at 5 mg/kg i.p. and i.p. injection of vehicle.

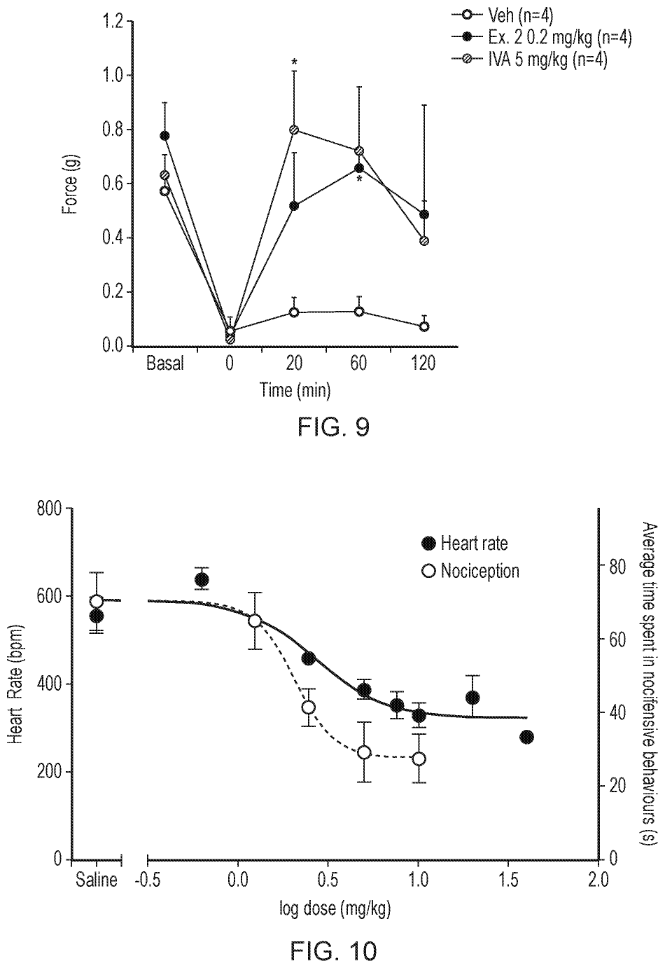

The compound of Example 2 delivered full analgesia at 0.2 mg/kg i.p. (FIG. 9, “883”).

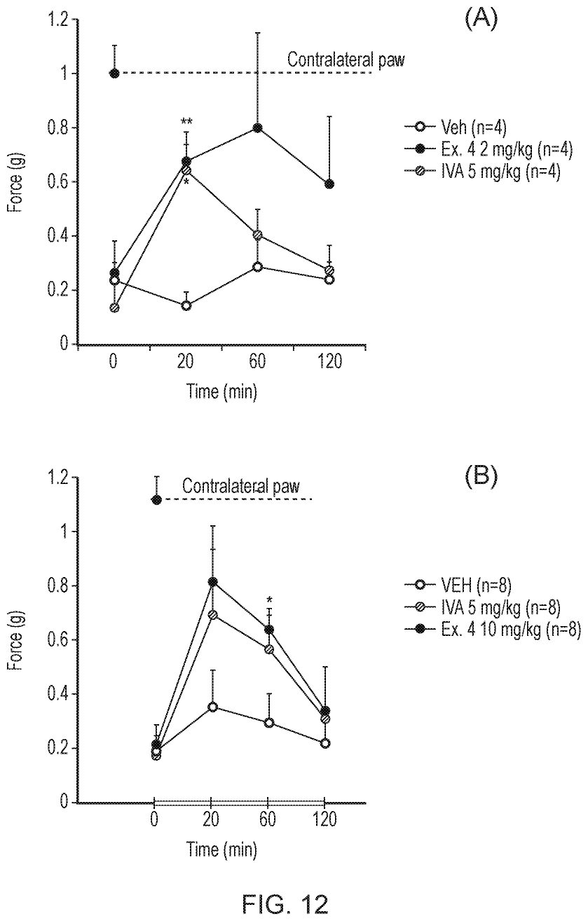

The compound of Example 4 gave maximal analgesia at 2 mg/kg i.p. ((A) in FIG. 12, “797”) and 10 mg/kg ((B) in FIG. 12, “797”).

No hyperalgesia observed in contralateral (unoperated) hind paw of the mice (see dotted line FIG. 12).

### Example 58: Bradycardia Testing

Test compounds were administered i.p. to awake, behaving, Black6 strain mice together with a vehicle only control arm. Heart rate in the mice were measured with MouseOx pulse oximeter.

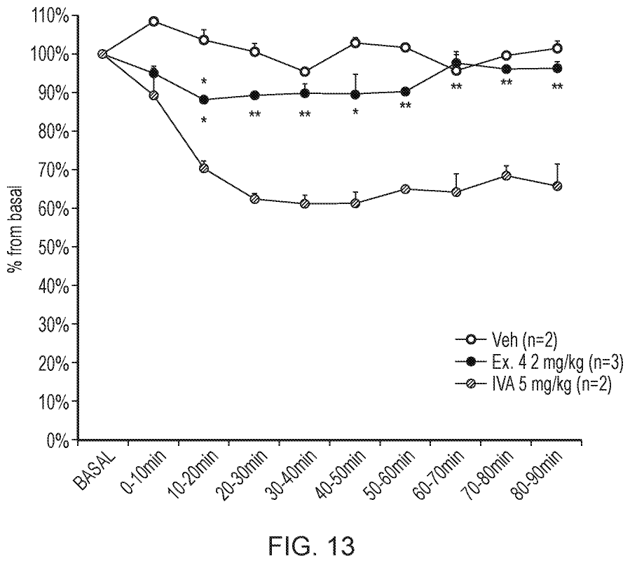

The compound of Example 4 is more than 19 times selective for HCN2 over HCN4 in the PatchXpress protocol (PX) assay described herein. As illustrated in Example 57, the compound of Example 4 provided maximal analgesia at a dose of 2 mg/kg. At this dose the compound produced minimal bradycardia in the mice and significantly lower bradycardia than ivabradine administered at a dose of 5 mg/kg i.p. (FIG. 13).

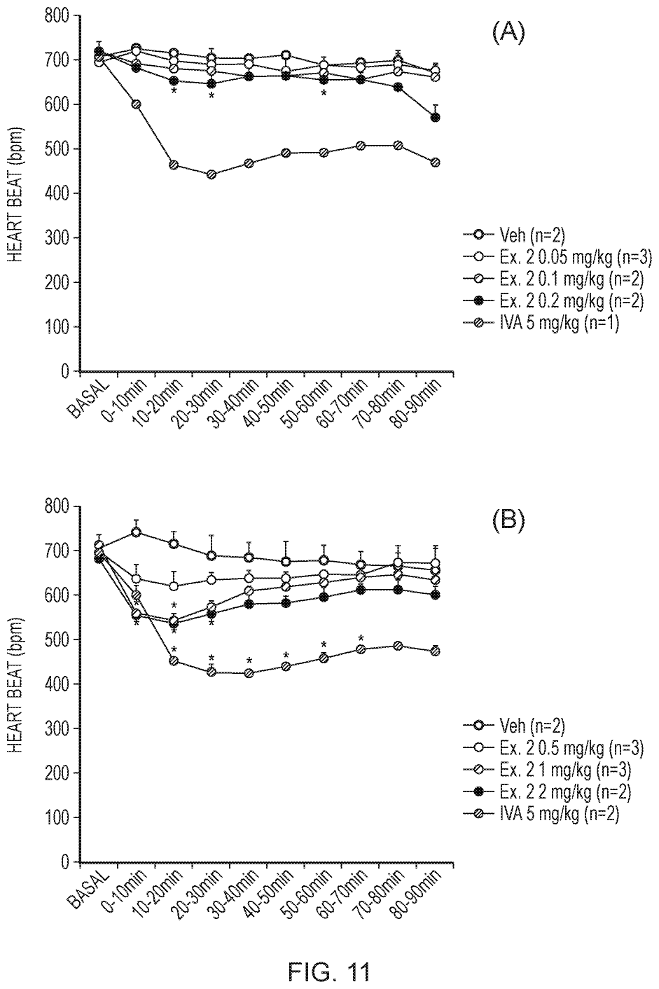

The compound of Example 2 was administered to awake, behaving, Black6 strain mice at doses ranging from 0.05 mg/kg to 2 mg/kg. The effect of the compound of Example 2 on heart beat is shown in FIG. 11 compared to ivabradine administered at a dose of 5 mg/kg i.p, and a vehicle control.

The compound of Example 2 is 21 times selective for HCN2 over HCN4 in the PatchXpress protocol (PX) assay described herein. As illustrated in Example 57 and FIG. 9, the compound of Example 2 provided full analgesia at a dose of 0.2 mg/kg i.p. At this dose the compound produced minimal bradycardia in the mice tested (see FIG. 11, panel (A)).

In contrast ivabradine blocks pain but because ivabradine does not discriminate between HCN2 and HCN4, there is little therapeutic window between analgesia and bradycardia in Black6 mice (FIG. 10). The data shown in FIG. 10 is based on inflammatory pain measured in a formalin model implemented in Black6 mice. The heart rate of the mice was measured using MouseOx pulse oximeter in awake, behaving mice (published data from Young G T et al. (2014), Pain 155: 1708-1719).

1. A compound of the formula (I), or a pharmaceutically acceptable salt thereof: 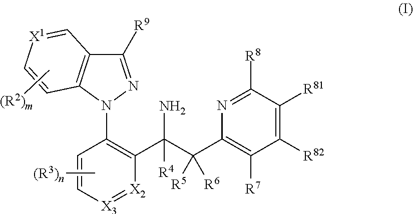
wherein
X¹ is N or CR¹;
R¹ is selected from: H, halo, —CN, C₁₋₆ alkyl, C₁₋₆ haloalkyl, —OR^{B1}, C₂₋₆ alkenyl, C₂₋₆ alkynyl, C₃₋₆ cycloalkyl and C₃₋₆ cycloalkyl-C₁₋₆ alkyl-, and wherein any alkyl, alkenyl, alkynyl or cycloalkyl group in R¹ is optionally substituted with 1 to 4 substituents independently selected from halo, C₁₋₄ alkyl, C₁₋₄ haloalkyl and —OR^{B2};
R² is independently at each occurrence selected from: halo, C₁₋₆ alkyl and C₁₋₆ haloalkyl;
X² is N or CR³²;
X³ is N or CR³³;
R³² and R³³ are independently selected from: H, halo, —CN, C₁₋₆ alkyl, C₁₋₆ haloalkyl, —NR^{A3}R^{A3} and —OR^{B3};
R³ is independently at each occurrence selected from: halo, —CN, C₁₋₆ alkyl, C₁₋₆ haloalkyl, —NR^{A3}R^{A3} and —OR^{B3};
R⁴, R⁵ and R⁶ are each independently selected from: H and C₁₋₄ alkyl,
or R⁵ and R⁶ together with the carbon atom to which they are attached form a C₃₋₆ cycloalkyl;
R⁷ is selected from: H, halo, —CN, C₁₋₆ alkyl, C₁₋₆ haloalkyl, C₃₋₆ cycloalkyl, —C(O)NR^{A4}R^{A4}, —N(R^{A4})C(O)R^{B4} and —C(O)R^{B4} and;
R⁸ is independently at each occurrence selected from: H, halo, —CN, nitro, C₁₋₆ alkyl, C₁₋₆ haloalkyl, C₂₋₆ alkenyl, C₂₋₆ alkynyl, —OR¹⁰, —NR¹⁰R¹¹, —S(O)ₓR¹⁰, —C(O)R¹⁰, —OC(O)R¹⁰, —C(O)OR^{10A}, —C(O)NR¹⁰R¹¹, —N(R¹¹)C(O)R¹⁰, —N(R¹¹)C(O)NR¹⁰R¹¹, —N(R¹¹)C(O)OR¹⁰, —N(R¹¹) SO₂R¹⁰, —SO₂NR¹⁰R¹¹, C₃₋₆ cycloalkyl, 3 to 7 membered heterocyclyl, phenyl and or 6 membered heteroaryl; wherein said alkyl, alkenyl, alkynyl, cycloalkyl, or heterocyclyl group is optionally substituted with from 1 to 4 R¹² groups, and said phenyl or heteroaryl group is optionally substituted with from 1 to 4 R¹³ groups;
R⁸¹ and R⁸² are each independently selected from: H, halo, —CN, C₁₋₄ alkyl, C₁₋₄ haloalkyl, —OH and —OR^{B8};
R⁹ is selected from H, halo, —CN and C₁₋₆ alkyl;
R¹⁰ is independently at each occurrence selected from: H, C₁₋₆ alkyl, C₁₋₆ haloalkyl and C₃₋₆ cycloalkyl; wherein said alkyl or cycloalkyl group is optionally substituted with from 1 to 4 R¹⁴ groups;
R^{10A} is selected from: C₁₋₆ alkyl, C₁₋₆ haloalkyl and C₃₋₆ cycloalkyl; wherein said alkyl or cycloalkyl group is optionally substituted with from 1 to 4 R¹⁴ groups;
R¹¹ is independently at each occurrence selected from: H and C₁₋₆ alkyl;
or R¹⁰ and R¹¹ together with the nitrogen to which they are attached form a 4 to 7 membered heterocyclyl, wherein said heterocyclyl is optionally substituted with 1 or 2 substituents selected from halo, ═O, C₁₋₄ alkyl, C₁₋₄ haloalkyl and —OR^{B7};
R¹² and R¹⁴ are each independently at each occurrence selected from: halo, ═O, —CN, nitro, C₁₋₄ alkyl, C₁₋₄ haloalkyl, C₃₋₆ cycloalkyl, —OR^{B5}, —NR^{A5}R^{A5}, —S(O)ₓR^{B5}, —C(O)R^{B5}, —NR^{A5}C(O)R^{B5}, —C(O)NR^{A5}R^{A5}, —NR^{A5}SO₂R^{B5} and —SO₂NR^{A5}R^{A5};
R¹³ is independently at each occurrence selected from: halo, —CN, nitro, C₁₋₄ alkyl, C₁₋₄ haloalkyl, C₃₋₆ cycloalkyl, —OR^{B6}, —NR^{A6}R^{A6}, —S(O)ₓR^{B6}, —C(O)R^{A6}, —NR^{A6}C(O)R^{B6}, —C(O)NR^{A6}R^{A6}, —NR^{A6}SO₂R^{B6}, —SO₂NR^{A6}R^{A6};
R^{B1} is independently at each occurrence selected from: H and C₁₋₆ alkyl;
R^{B3} are independently at each occurrence selected from: H, C₁₋₆ alkyl and C₁₋₆ haloalkyl;
R^{B2}, R^{B4}, R^{B5}, R^{B6}, R^{B7} are independently at each occurrence selected from: H, C₁₋₄ alkyl and C₁₋₄ haloalkyl;
R^{B8} is independently at each occurrence selected from: C₁₋₄ alkyl and C₁₋₄ haloalkyl;
R^{A3}, R^{A4}, R^{A5} and R^{A6} are independently at each occurrence selected from H and C₁₋₄ alkyl;
m is an integer selected from: 0, 1, 2 and 3;
n is an integer selected from: 0, 1 or 2; and
x is independently at each occurrence an integer selected from 0, 1, 2 and 3;
provided that:
(i) when X¹ is N, then X₂ is CR³² and X₃ is CR³³;
(ii) when X¹ is CR¹ and R¹ is —CN, then X₂ is CR³² and X₃ is CR³³;
(iii) when X¹ is CR¹ and R¹ is —CF₃, then R⁸ is not —SO₂Me; and
(iv) X² and X³ are not both N.

2. The compound of claim 1, wherein R⁷ is selected from: H, halo and C₁₋₄ alkyl.

3. The compound of claim 1 or claim 2, wherein R⁸ is selected from: H, halo, —CN, C₁₋₄ alkyl, C₁₋₄ haloalkyl, —C₁₋₄ alkyl-OR^{B5}, —OH, —OC₁₋₄ alkyl, —OC₂₋₄ alkyl-OR^{B5}, —C(O)C₁₋₄ alkyl, —S(O)₂C₁₋₄ alkyl, —C(O)NH₂, —C(O)N(H)C₁₋₄ alkyl and —C(O)N(C₁₋₄ alkyl)₂.

4. The compound of claim 1 or claim 2, wherein R⁸ is selected from: H, —CN and —S(O)₂C₁₋₄ alkyl (optionally wherein R⁸ is selected from —CN and —S(O)₂Me).

5. The compound of claim 1, wherein R⁸ is selected from: H, —CN and —S(O)₂C₁₋₄ alkyl; and R⁷ is selected from: H, halo and C₁₋₄ alkyl, provided that R⁷ and R⁸ are not both H.

6. The compound of claim 1, wherein R⁷ is selected from fluoro and methyl and R⁸ is selected from H, —CN, C₁₋₄ alkyl, —C₁₋₃ alkyl-OH, —C₁₋₃ alkyl-OMe and —S(O)₂C₁₋₄ alkyl (optionally wherein R⁸ is selected from H, —CN, C₁₋₃ alkyl and —S(O)₂C₁₋₃ alkyl).

7. The compound of any one of claims 1 to 6, wherein R⁸¹ and R⁸² are independently selected from: H, halo, C₁₋₄ alkyl, C₁₋₄ haloalkyl, —OC₁₋₄ alkyl and —OC₁₋₄ haloalkyl.

8. The compound of any one of claims 1 to 6, wherein R⁸¹ is halo (e.g. F) and R⁸² is H.

9. The compound of claim 1, wherein the group 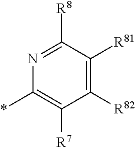
is selected from: 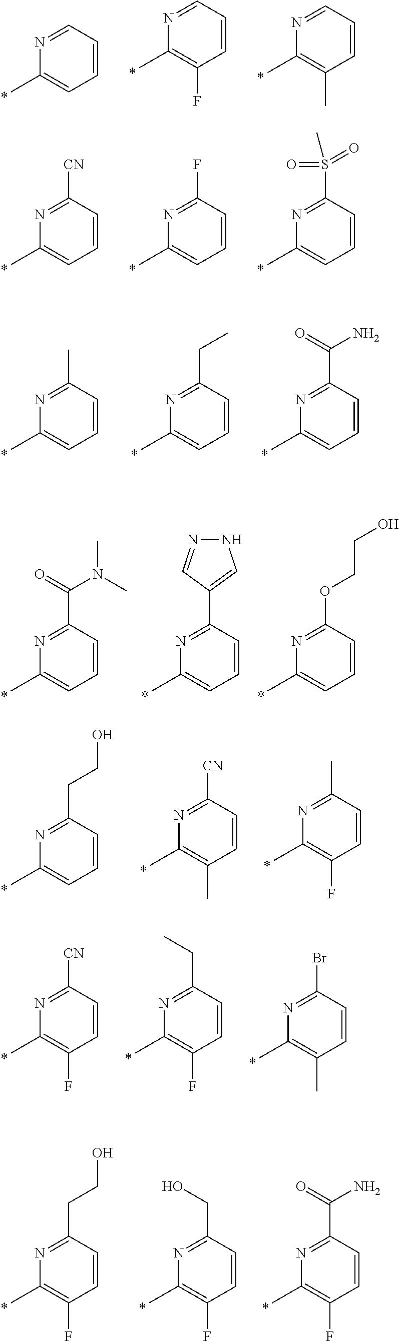 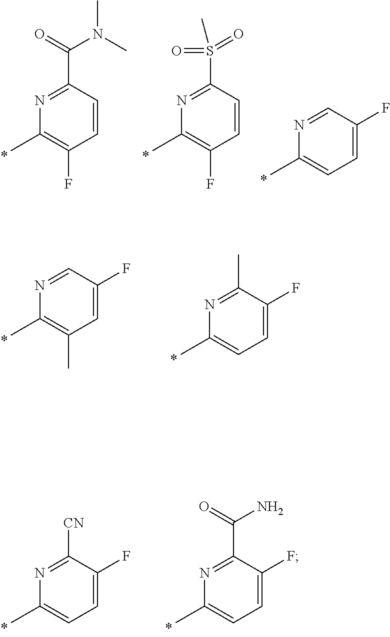
wherein * shows the point of attachment to the remainder of the molecule.

10. The compound of any one of claims 1 to 9, wherein X₂ is CR³² and X₃ is CR³³ (optionally wherein R³² and R³³ are H).

11. The compound of any one of claims 1 to 9, wherein X₂ is N and X₃ is CR³³.

12. The compound of claim 11, wherein R³³ selected from H, halo and C₁₋₃ alkyl (optionally wherein R³³ is selected from halo and C₁₋₃ alkyl).

13. The compound of any one of claims 1 to 10, wherein X, is N.

14. The compound of any one of claims 1 to 12, wherein X, is CR¹ and R¹ is selected from: H, halo, —CN, C₁₋₄ alkyl, C₁₋₄ haloalkyl and —ORB, and wherein said alkyl group in R¹ is optionally substituted with —OR^{B2}.

15. The compound of any one of claims 1 to 12, wherein X, is CR¹ and R¹ is selected from: H, halo, —CN, C₁₋₃ alky and C₁₋₃ haloalkyl.

16. The compound of any one of claims 1 to 12, wherein X, is CH.

17. The compound of any one of claims 1 to 16, wherein n is 0.

18. The compound of any one of claims 1 to 17, wherein m is 0.

19. The compound of any one of claims 1 to 18, wherein R⁹ is selected from: H and C₁₋₄ alkyl (optionally wherein R⁹ is H).

20. The compound of any one of claims 1 to 19, wherein R⁴ and R⁵ are H and R⁶ is H or C₁₋₃ alkyl (optionally wherein R⁴, R⁵ and R⁶ are H).

21. The compound of any of claims 1 to 20, wherein the group 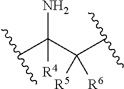
is of the formula: 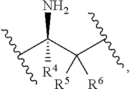
optionally wherein the group 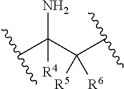
is:

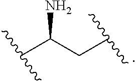 [SMILES: *C[C@@H](*)N]

22. A compound of claim 1 selected from a compound shown in Table 1 in the description, or a pharmaceutically acceptable salt thereof.

23. A pharmaceutical composition comprising a compound of any of claims 1 to 22, or a pharmaceutically acceptable salt thereof, and a pharmaceutically acceptable excipient.

24. A compound of any of claims 1 to 22, or a pharmaceutically acceptable salt thereof, for use as a medicament.

25. A compound of any of claims 1 to 22, or a pharmaceutically acceptable salt thereof, for use in the treatment of a disease or medical condition mediated by hyperpolarisation activated cyclic-nucleotide modulated ion channel 2 (HCN2).

26. A method of treating a disease or medical condition mediated by HCN2 in a subject in need thereof, the method comprising administering to the subject an effective amount of: a compound of any of claims 1 to 22, or a pharmaceutically acceptable salt thereof.

27. The compound for the use of claim 25, or the method of treatment of claim 26, wherein the disease or medical condition mediated by HCN2 is pain, for example neuropathic pain or inflammatory pain (for example wherein the pain is peripheral neuropathic pain).

28. A compound of any of claims 1 to 22, or a pharmaceutically acceptable salt thereof, for use in the treatment of tinnitus or a related condition.

29. A HCN2 inhibitor for use in the treatment of migraine.

30. The HCN2 inhibitor for the use of claim 29, wherein the HCN2 inhibitor is a compound of any of claims 1 to 22, or a pharmaceutically acceptable salt thereof.
